SEQUENCE LISTING (161 sequences)

SEQ ID NO: 1 - protein, 8 aa, synthetic construct
  REGION 1..8: note = Synthetic: hu1MU32A, hu1MU8A, HCDR1
GYTFTSYW

SEQ ID NO: 2 - protein, 8 aa, synthetic construct
  REGION 1..8: note = Synthetic: hu1MU32A, HCDR2
IHPSDSET

SEQ ID NO: 3 - protein, 14 aa, synthetic construct
  REGION 1..14: note = Synthetic: hu1MU32A, hu1MU8A, HCDR3
ARQGIITSVQEFAY

SEQ ID NO: 4 - protein, 5 aa, synthetic construct
  REGION 1..5: note = Synthetic: hu1MU32A, hu1MU8A, LCDR1
SSVNY

SEQ ID NO: 5 - skipped

SEQ ID NO: 6 - protein, 9 aa, synthetic construct
  REGION 1..9: note = Synthetic: hu1MU32A, hu1MU8A, LCDR3
QQYHSYPLT

SEQ ID NO: 7 - protein, 121 aa, synthetic construct
  REGION 1..121: note = Synthetic: hu1MU32A, VH
QVQLVQSGAEVKKPGSSVKVSCKASGYTFTSYWMNWVRQAPGQCLEWMGMIHPSDSETRLNQKFKDRVTITADKSTSTAYMELSSLRSEDTAVYYCARQGIITSVQEFAYWGQGTLVTVSS

SEQ ID NO: 8 - protein, 105 aa, synthetic construct
  REGION 1..105: note = Synthetic: hu1MU32A, VL
IQMTQSPSSLSASVGDRVTITCSASSSVNYIYWYQQKPGKAPKLLIYRTSNLASGVPSRFSGSGSGTDFTLTISSLQPEDFATYYCQQYHSYPLTFGGGTKVEIK

SEQ ID NO: 9 - protein, 8 aa, synthetic construct
  REGION 1..8: note = Synthetic: hu1MU11A, HCDR1
GYEFSSHW

SEQ ID NO: 10 - protein, 8 aa, synthetic construct
  REGION 1..8: note = Synthetic: hu1MU11A, HCDR2
IYPGDGDI

SEQ ID NO: 11 - protein, 10 aa, synthetic construct
  REGION 1..10: note = Synthetic: hu1MU11A, HCDR3
ARHGNYVMDY

SEQ ID NO: 12 - protein, 5 aa, synthetic construct
  REGION 1..5: note = Synthetic: hu1MU11A, LCDR1
SSVSY

SEQ ID NO: 13 - protein, 9 aa, synthetic construct
  REGION 1..9: note = Synthetic: hu1MU11A, LCDR3
QQFHDYPRT

SEQ ID NO: 14 - protein, 117 aa, synthetic construct
  REGION 1..117: note = Synthetic: hu1MU11A, VH
EVQLVQSGAEVKKPGESLKISCKGSGYEFSSHWMNWVRQMPGKGLEWMGQIYPGDGDINYNEKFRGQVTISADKSISTAYLQWSSLKASDTAMYYCARHGNYVMDYWGQGTLVTVSS

SEQ ID NO: 15 - protein, 105 aa, synthetic construct
  REGION 1..105: note = Synthetic: hu1MU11A, VL
IQLTQSPSFLSASVGDRVTITCSASSSVSYMFWYQQKPGKAPKPWIYRTSNLASGVPSRFSGSGSGTEFTLTISSLQPEDFATYYCQQFHDYPRTFGGGTKVEIK

SEQ ID NO: 16 - protein, 8 aa, synthetic construct
  REGION 1..8: note = Synthetic: hu1MU8A, HCDR 2
IHPSDSES

SEQ ID NO: 17 - protein, 121 aa, synthetic construct
  REGION 1..121: note = Synthetic: hu1MU8A, VH
QVQLVQSGAEVKKPGSSVKVSCKASGYTFTSYWLNWVRQAPGQGLEWMGMIHPSDSESRLNQKFKDRVTITADKSTSTAYMELSSLRSEDTAVYYCARQGIITSVQEFAYWGQGTLVTVSS

SEQ ID NO: 18 - protein, 105 aa, synthetic construct
  REGION 1..105: note = Synthetic: hu1MU8A, VL
IQMTQSPSSLSASVGDRVTITCSASSSVNYIFWYQQKPGKAPKLLIYRTSNLASGVPSRFSGSGSGTDFTLTISSLQPEDFATYYCQQYHSYPLTFGGGTKVEIK

SEQ ID NO: 19 - protein, 8 aa, synthetic construct
  REGION 1..8: note = Synthetic: hu1MU37, HCDR1
GFTFSSFG

SEQ ID NO: 20 - protein, 8 aa, synthetic construct
  REGION 1..8: note = Synthetic: hu1MU37, HCDR2
ISSGSSTI

SEQ ID NO: 21 - protein, 13 aa, synthetic construct
  REGION 1..13: note = Synthetic: hu1MU37, HCDR3
ARWGYYGSSYFAY

SEQ ID NO: 22 - protein, 6 aa, synthetic construct
  REGION 1..6: note = Synthetic: hu1MU37, LCDR1
EDIYNR

SEQ ID NO: 23 - skipped

SEQ ID NO: 24 - protein, 9 aa, synthetic construct
  REGION 1..9: note = Synthetic: hu1MU37, LCDR3
QQFWRTPPT

SEQ ID NO: 25 - protein, 120 aa, synthetic construct
  REGION 1..120: note = Synthetic: hu1MU37, VH
EVQLVESGGGLVQPGGSLRLSCAASGFTFSSFGMHWVRQAPGKGLEWVSYISSGSSTIYYADTVKGRFTISRDNAKNSLYLQMNSLRAEDTAVYYCARWGYYGSSYFAYWGQGTLVTVSS

SEQ ID NO: 26 - protein, 107 aa, synthetic construct
  REGION 1..107: note = Synthetic: hu1MU37, VL
DIQMTQSPSSLSASVGDRVTITCKASEDIYNRLAWYQQKPGKAPKPLISGATNLETGVPSRFSGSGSGTDFTFTISSLQPEDIATYYCQQFWRTPPTFGGGTKVEIK

SEQ ID NO: 27 - protein, 8 aa, synthetic construct
  REGION 1..8: note = Synthetic: HuS_anti-Muc17, HCDR1
GGSFSGYY

SEQ ID NO: 28 - protein, 7 aa, synthetic construct
  REGION 1..7: note = Synthetic: HuS_anti-Muc17, HCDR2
IDASGST

SEQ ID NO: 29 - protein, 14 aa, synthetic construct
  REGION 1..14: note = Synthetic: HuS_anti-Muc17, HCDR3
ARKKYSTVWSYFDN

SEQ ID NO: 30 - protein, 6 aa, synthetic construct
  REGION 1..6: note = Synthetic: HuS_anti-Muc17, LCDR1
KLGDKY

SEQ ID NO: 31 - skipped

SEQ ID NO: 32 - protein, 9 aa, synthetic construct
  REGION 1..9: note = Synthetic: HuS_anti-Muc17, LCDR3
QAWGSSTAV

SEQ ID NO: 33 - protein, 120 aa, synthetic construct
  REGION 1..120: note = Synthetic: HuS_anti-Muc17, VH
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 34 - protein, 106 aa, synthetic construct
  REGION 1..106: note = Synthetic: HuS_anti-Muc17, VL
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 35 - protein, 8 aa, synthetic construct
  REGION 1..8: note = Synthetic: huD139A, HCDR1
GYTFTDYF

SEQ ID NO: 36 - protein, 8 aa, synthetic construct
  REGION 1..8: note = Synthetic: huD139A, HCDR2
INPYNDIT

SEQ ID NO: 37 - protein, 15 aa, synthetic construct
  REGION 1..15: note = Synthetic: huD139A, HCDR3
AREGVLYDGYYEGAY

SEQ ID NO: 38 - protein, 6 aa, synthetic construct
  REGION 1..6: note = Synthetic: huD139A, LCDR1
QNVGIA

SEQ ID NO: 39 - skipped

SEQ ID NO: 40 - protein, 9 aa, synthetic construct
  REGION 1..9: note = Synthetic: huD139A, LCDR3
QQYSTYPYT

SEQ ID NO: 41 - protein, 122 aa, synthetic construct
  REGION 1..122: note = Synthetic: huD139A, VH
QVQLVQSGAEVKKPGASVKVSCKASGYTFTDYFMNWVRQAPGQCLEWMGVINPYNDITIYNQKFQGRVTMTVDRSTSTVYMELSSLRSEDTAVYYCAREGVLYDGYYEGAYWGQGTLVTVSS

SEQ ID NO: 42 - protein, 107 aa, synthetic construct
  REGION 1..107: note = Synthetic: huD139A, VL
DIQLTQSPSFLSASVGDRVTITCKASQNVGIAVAWYQQKPGKAPKLLIYAASNRYTGVPSRFSGSGSGTEFTLTISSLQPEDFATYYCQQYSTYPYTFGCGTKLEIK

SEQ ID NO: 43 - protein, 8 aa, synthetic construct
  REGION 1..8: note = Synthetic: hu1D4, HCDR1
GFTFSSYG

SEQ ID NO: 44 - protein, 8 aa, synthetic construct
  REGION 1..8: note = Synthetic: hu1D4, HCDR2
ISHHGSSK

SEQ ID NO: 45 - protein, 11 aa, synthetic construct
  REGION 1..11: note = Synthetic: hu1D4, HCDR3
ARDWFFYLFDY

SEQ ID NO: 46 - protein, 11 aa, synthetic construct
  REGION 1..11: note = Synthetic: hu1D4, LCDR1
QSLLHSDAKTF

SEQ ID NO: 47 - skipped

SEQ ID NO: 48 - protein, 9 aa, synthetic construct
  REGION 1..9: note = Synthetic: hu1D4, LCDR3
LQGERLPFT

SEQ ID NO: 49 - protein, 118 aa, synthetic construct
  REGION 1..118: note = Synthetic: hu1D4, VH
EVQLLESGGGLVQPGGSLRLSCAASGFTFSSYGMHWVRQAPGKGLEWVAVISHHGSSKYYADSVKGRFTISRDNSKNTLYLQMNSLRAEDTAVYYCARDWFFYLFDYWGQGTLVTVSS

SEQ ID NO: 50 - protein, 112 aa, synthetic construct
  REGION 1..112: note = Synthetic: hu1D4, VL
DIQMTQSPSSLSASVGDRVTITCKSSQSLLHSDAKTFLYWYQQKPGKAPKLLIYEVSNRFSGVPSRFSGSGSGTDFTLTISSLQPEDFATYYCLQGERLPFTFGQGTKVEIK

SEQ ID NO: 51 - protein, 8 aa, synthetic construct
  REGION 1..8: note = Synthetic: huD143, HCDR1
DYIFSNYY

SEQ ID NO: 52 - protein, 8 aa, synthetic construct
  REGION 1..8: note = Synthetic: huD143, HCDR2
ILPGTGNT

SEQ ID NO: 53 - protein, 11 aa, synthetic construct
  REGION 1..11: note = Synthetic: huD143, huD138C, HCDR3
ARWGDYALFAN

SEQ ID NO: 54 - protein, 6 aa, synthetic construct
  REGION 1..6: note = Synthetic: huD143, huD138C, LCDR1
QNVGTN

SEQ ID NO: 55 - skipped

SEQ ID NO: 56 - protein, 9 aa, synthetic construct
  REGION 1..9: note = Synthetic: huD143, LCDR3
QQYNNYPLT

SEQ ID NO: 57 - protein, 118 aa, synthetic construct
  REGION 1..118: note = Synthetic: huD143, VH
QVQLVQSGAEVKKPGASVKVSCKATDYIFSNYYIEWVRQAPGQGLEWMGEILPGTGNTVYNEKFKDRVTMTVDTSTSTVYMELSSLRSEDTAVYYCARWGDYALFANWGQGTLVTVSS

SEQ ID NO: 58 - protein, 107 aa, synthetic construct
  REGION 1..107: note = Synthetic: huD143, VL
DIQMTQSPSFLSASVGDRVTITCKASQNVGTNVAWYQQKPGKAPKPLIYSTSYRYSGVPSRFSGSGSGTEFTLTISSLQPEDFATYYCQQYNNYPLTFGGGTKVEIK

SEQ ID NO: 59 - protein, 8 aa, synthetic construct
  REGION 1..8: note = Synthetic: huD138C, HCDR1
DYTFSNYY

SEQ ID NO: 60 - protein, 8 aa, synthetic construct
  REGION 1..8: note = Synthetic: huD138C, HCDR2
ILPGNGNT

SEQ ID NO: 61 - skipped

SEQ ID NO: 62 - protein, 9 aa, synthetic construct
  REGION 1..9: note = Synthetic: huD138C, LCDR3
QQYNSYPFT

SEQ ID NO: 63 - protein, 118 aa, synthetic construct
  REGION 1..118: note = Synthetic: huD138C, VH
QVQLVQSGAEVKKPGASVKVSCKASDYTFSNYYIEWVRQAPGQGLEWMGEILPGNGNTVYNEKFKDRVTMTVDTSTSTAYMELRSLRSDDTAVYYCARWGDYALFANWGQGTLVTVSS

SEQ ID NO: 64 - protein, 107 aa, synthetic construct
  REGION 1..107: note = Synthetic: huD138C, VL
DIQMTQSPSTLSASVGDRVTITCKASQNVGTNVAWYQQKPGKAPKALIYSASYRYSGVPSRFSGSGSGTEFTLTISSLQPDDFATYYCQQYNSYPFTFGQGTKLEIK

SEQ ID NO: 65 - protein, 8 aa, synthetic construct
  REGION 1..8: note = Synthetic: huCldn29B, HCDR1
GYTFTNSG

SEQ ID NO: 66 - protein, 8 aa, synthetic construct
  REGION 1..8: note = Synthetic: huCldn29B, HCDR2
INTNTGEP

SEQ ID NO: 67 - protein, 11 aa, synthetic construct
  REGION 1..11: note = Synthetic: huCldn29B, HCDR3
ARYYYGNSFAY

SEQ ID NO: 68 - protein, 12 aa, synthetic construct
  REGION 1..12: note = Synthetic: huCldn29B, huCldn20, LCDR1
QSLLNSGNQKNY

SEQ ID NO: 69 - skipped

SEQ ID NO: 70 - protein, 9 aa, synthetic construct
  REGION 1..9: note = Synthetic: huCldn29B, LCDR3
QNNYFYPLT

SEQ ID NO: 71 - protein, 118 aa, synthetic construct
  REGION 1..118: note = Synthetic: huCldn29B, VH
QVQLVQSGAEVKKPGASVKVSCKASGYTFTNSGMNWVRQAPGQGLEWMGWINTNTGEPTFAEEFRGRVTMTRDTSISTAYMELSRLRSDDTAVYYCARYYYGNSFAYWGQGTLVTVSS

SEQ ID NO: 72 - protein, 113 aa, synthetic construct
  REGION 1..113: note = Synthetic: huCldn29B, VL
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNNYFYPLTFGGGTKVEIK

SEQ ID NO: 73 - protein, 8 aa, synthetic construct
  REGION 1..8: note = Synthetic: huCldn16A, HCDR1
GYTFTNFG

SEQ ID NO: 74 - protein, 8 aa, synthetic construct
  REGION 1..8: note = Synthetic: huCldn16A, HCDR2
IYPSSGNT

SEQ ID NO: 75 - protein, 14 aa, synthetic construct
  REGION 1..14: note = Synthetic: huCldn16A, HCDR3
ARGGGPLRSRYFDY

SEQ ID NO: 76 - protein, 12 aa, synthetic construct
  REGION 1..12: note = Synthetic: huCldn16A, LCDR1
QSLFSSGNQKNY

SEQ ID NO: 77 - protein, 9 aa, synthetic construct
  REGION 1..9: note = Synthetic: huCldn16A, LCDR3
QNDYYYPLT

SEQ ID NO: 78 - protein, 121 aa, synthetic construct
  REGION 1..121: note = Synthetic: huCldn16A, VH
QVQLVQSGAEVKKPGASVKVSCKASGYTFTNFGITWVRQAPGQGLEWMGEIYPSSGNTFYNEKFKGRVTMTTDTSTSTAYMELRSLRSDDTAVYYCARGGGPLRSRYFDYWGQGTLVTVSS

SEQ ID NO: 79 - protein, 113 aa, synthetic construct
  REGION 1..113: note = Synthetic: huCldn16A, VL
DIVMTQSPDSLAVSLGERATINCRSSQSLFSSGNQKNYLTWYQQKPGQPPKLLIYWASTRESGVPDRFSGSGSGTDFTLTISSLQAEDVAVYYCQNDYYYPLTFGGGTKVEIK

SEQ ID NO: 80 - protein, 8 aa, synthetic construct
  REGION 1..8: note = Synthetic: huCldn20, HCDR1
GFTFSSFG

SEQ ID NO: 81 - protein, 8 aa, synthetic construct
  REGION 1..8: note = Synthetic: huCldn20, HCDR2
ISSGNSAI

SEQ ID NO: 82 - protein, 11 aa, synthetic construct
  REGION 1..11: note = Synthetic: huCldn20, HCDR3
ARLRYGNSFDY

SEQ ID NO: 83 - protein, 9 aa, synthetic construct
  REGION 1..9: note = Synthetic: huCldn20, LCDR3
QNNYYYPLT

SEQ ID NO: 84 - protein, 118 aa, synthetic construct
  REGION 1..118: note = Synthetic: huCldn20, VH
EVQLVESGGGLVQPGGSLRLSCAASGFTFSSFGMHWVRQAPGKGLEWVSYISSGNSAIYYADTVNGRFTISRDNAKNSLYLQMNSLRAEDTAVYYCARLRYGNSFDYWGQGTLVTVSS

SEQ ID NO: 85 - protein, 113 aa, synthetic construct
  REGION 1..113: note = Synthetic: huCldn20, VL
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNNYYYPLTFGGGTKVEIK

SEQ ID NO: 86 - protein, 8 aa, synthetic construct
  REGION 1..8: note = Synthetic: huCldn23A, HCDR1
GYAFNNYW

SEQ ID NO: 87 - protein, 8 aa, synthetic construct
  REGION 1..8: note = Synthetic: huCldn23A, HCDR2
ISPGNGNS

SEQ ID NO: 88 - protein, 12 aa, synthetic construct
  REGION 1..12: note = Synthetic: huCldn23A, HCDR3
ARGGRYGNAMDY

SEQ ID NO: 89 - protein, 12 aa, synthetic construct
  REGION 1..12: note = Synthetic: huCldn23A, LCDR1
QSLLNSGNQRNY

SEQ ID NO: 90 - protein, 9 aa, synthetic construct
  REGION 1..9: note = Synthetic: huCldn23A, LCDR3
QNAYFYPYT

SEQ ID NO: 91 - protein, 119 aa, synthetic construct
  REGION 1..119: note = Synthetic: huCldn23A, VH
QVQLVQSGAEVKKPGSSVKVSCKASGYAFNNYWMNWVRQAPGQGLEWMGQISPGNGNSNFNGKFKGRVTITADKSTSTAYMELSSLRSEDTAVYYCARGGRYGNAMDYWGQGTTVTVSS

SEQ ID NO: 92 - protein, 113 aa, synthetic construct
  REGION 1..113: note = Synthetic: huCldn23A, VL
DIVMTQSPDSLAVSLGERATINCKSSQSLLNSGNQRNYLTWYQQKPGQPPKLLIYWASTRESGVPDRFSGSGSGTDFTLTISSLQAEDVAVYYCQNAYFYPYTFGGGTKVEIK

SEQ ID NO: 93 - protein, 8 aa, synthetic construct
  REGION 1..8: note = Synthetic: aCD3 (VH_A116E), aCD3 (VH_A116E, Y60A), aCD3 (VH_A116E, K59E, Y60A), aCD3 (VH_A116D), HCDR1
GFTFSTYA

SEQ ID NO: 94 - protein, 10 aa, synthetic construct
  REGION 1..10: note = Synthetic: aCD3 (VH_A116E), aCD3 (VH_A116D), HCDR2
IRSKYNNYAT

SEQ ID NO: 95 - protein, 16 aa, synthetic construct
  REGION 1..16: note = Synthetic: aCD3 (VH_A116E), aCD3 (VH_A116E, Y60A), aCD3 (VH_A116E, K59E, Y60A), HCDR3
VRHGNFGDSYVSWFEY

SEQ ID NO: 96 - protein, 9 aa, synthetic construct
  REGION 1..9: note = Synthetic: aCD3 (VH_A116E), aCD3 (VH_A116E, Y60A), aCD3 (VH_A116E, K59E, Y60A), aCD3 (VH_A116D), LCDR1
TGAVTTSNY

SEQ ID NO: 97 - skipped

SEQ ID NO: 98 - protein, 9 aa, synthetic construct
  REGION 1..9: note = Synthetic: aCD3 (VH_A116E), aCD3 (VH_A116E, Y60A), aCD3 (VH_A116E, K59E, Y60A), aCD3 (VH_A116D), LCDR3
ALWYSNHWV

SEQ ID NO: 99 - protein, 125 aa, synthetic construct
  REGION 1..125: note = Synthetic: aCD3 (VH_A116E), VH
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSS

SEQ ID NO: 100 - protein, 109 aa, synthetic construct
  REGION 1..109: note = Synthetic: aCD3 (VH_A116E), VL aCD3 (VH_A116E, Y60A), VL aCD3 (VH_A116E, K59E, Y60A), VL aCD3 (VH_A116D), VL
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 101 - protein, 10 aa, synthetic construct
  REGION 1..10: note = Synthetic: aCD3 (VH_A116E, Y60A), HCDR2
IRSKANNYAT

SEQ ID NO: 102 - protein, 125 aa, synthetic construct
  REGION 1..125: note = Synthetic: aCD3 (VH_A116E, Y60A), VH
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSS

SEQ ID NO: 103 - protein, 10 aa, synthetic construct
  REGION 1..10: note = Synthetic: aCD3 (VH_A116E, K59E, Y60A), HCDR2
IRSEANNYAT

SEQ ID NO: 104 - protein, 125 aa, synthetic construct
  REGION 1..125: note = Synthetic: aCD3 (VH_A116E, K59E, Y60A), VH
EVQLVESGGGLVQPGGSLRLSCAASGFTFSTYAMNWVRQAPGKGLEWVGRIRSEANNYATYYADSVKGRFTISRDDSKNTLYLQMNSLRAEDTAVYYCVRHGNFGDSYVSWFEYWGQGTLVTVSS

SEQ ID NO: 105 - protein, 16 aa, synthetic construct
  REGION 1..16: note = Synthetic: aCD3 (VH_A116D), HCDR3
VRHGNFGDSYVSWFDY

SEQ ID NO: 106 - protein, 125 aa, synthetic construct
  REGION 1..125: note = Synthetic: aCD3 (VH_A116D), VH
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 107 - protein, 8 aa, synthetic construct
  REGION 1..8: note = Synthetic: aCD28 (VH_C55S), aCD28 (VH_C55S, Y109F, L111Y), aCD28 (VH_C55S, N62S, L111A), aCD28 (VH_C55S, N62S, Y109F, L111Y), aCD28 (VH_C55S, L111Y, W113F), aCD28 (VH_C55S, W113A), HCDR1
GYTFTSYY

SEQ ID NO: 108 - protein, 8 aa, synthetic construct
  REGION 1..8: note = Synthetic: aCD28 (VH_C55S), aCD28 (VH_C55S, Y109F, L111Y), aCD28 (VH_C55S, S36G, L111A), aCD28 (VH_C55S, S36G, Y109F, L111Y), aCD28 (VH_C55S, L111Y, W113F), aCD28 (VH_C55S, W113A), HCDR2
IYPGNVNT

SEQ ID NO: 109 - protein, 13 aa, synthetic construct
  REGION 1..13: note = Synthetic: aCD28 (VH_C55S), HCDR3
TRSHYGLDWNFDV

SEQ ID NO: 110 - protein, 6 aa, synthetic construct
  REGION 1..6: note = Synthetic: aCD28 (VH_C55S), aCD28 (VH_C55S, Y109F, L111Y), aCD28 (VH_C55S, S36G, L111A), aCD28 (VH_C55S, N62S, L111A), aCD28 (VH_C55S, S36G, Y109F, L111Y), aCD28 (VH_C55S, N62S, Y109F, L111Y), aCD28 (VH_C55S, L111Y, W113F), aCD28 (VH_C55S, W113A), LCDR1
QNIYVW

SEQ ID NO: 111 - skipped

SEQ ID NO: 112 - protein, 9 aa, synthetic construct
  REGION 1..9: note = Synthetic: aCD28 (VH_C55S), aCD28 (VH_C55S, Y109F, L111Y), aCD28 (VH_C55S, S36G, L111A), aCD28 (VH_C55S, N62S, L111A), aCD28 (VH_C55S, S36G, Y109F, L111Y), aCD28 (VH_C55S, N62S, Y109F, L111Y), aCD28 (VH_C55S, L111Y, W113F), aCD28 (VH_C55S, W113A), LCDR3
QQGQTYPYT

SEQ ID NO: 113 - protein, 120 aa, synthetic construct
  REGION 1..120: note = Synthetic: aCD28 (VH_C55S), VH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 114 - protein, 107 aa, synthetic construct
  REGION 1..107: note = Synthetic: aCD28 (VH_C55S), VL aCD28 (VH_C55S, Y109F, L111Y), VL aCD28 (VH_C55S, S36G, L111A), VL aCD28 (VH_C55S, N62S, L111A), VL aCD28 (VH_C55S, S36G, Y109F, L111Y), VL aCD28 (VH_C55S, N62S, Y109F, L111Y), VL aCD28 (VH_C55S, L111Y, W113F), VL aCD28 (VH_C55S, W113A), VL
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 115 - protein, 13 aa, synthetic construct
  REGION 1..13: note = Synthetic: aCD28 (VH_C55S, Y109F, L111Y), aCD28 (VH_C55S, S36G, Y109F, L111Y), aCD28 (VH_C55S, N62S, Y109F, L111Y), HCDR3
TRSHFGYDWNFDV

SEQ ID NO: 116 - protein, 120 aa, synthetic construct
  REGION 1..120: note = Synthetic: aCD28 (VH_C55S, Y109F, L111Y), VH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHFGYDWNFDVWGQGTTVTVSS

SEQ ID NO: 117 - protein, 8 aa, synthetic construct
  REGION 1..8: note = Synthetic: aCD28 (VH_C55S, S36G, L111A), aCD28 (VH_C55S, S36G, Y109F, L111Y), HCDR1
GYTFTGYY

SEQ ID NO: 118 - protein, 13 aa, synthetic construct
  REGION 1..13: note = Synthetic: aCD28 (VH_C55S, S36G, L111A), aCD28 (VH_C55S, N62S, L111A), HCDR3
TRSHYGADWNFDV

SEQ ID NO: 119 - protein, 120 aa, synthetic construct
  REGION 1..120: note = Synthetic: aCD28 (VH_C55S, S36G, L111A), VH
QVQLVQSGAEVKKPGASVKVSCKASGYTFTGYYIHWVRQAPGQGLEWIGSIYPGNVNTNYNEKFKDRATLTVDTSISTAYMELSRLRSDDTAVYFCTRSHYGADWNFDVWGQGTTVTVSS

SEQ ID NO: 120 - protein, 8 aa, synthetic construct
  REGION 1..8: note = Synthetic: aCD28 (VH_C55S, N62S, L111A), aCD28 (VH_C55S, N62S, Y109F, L111Y), HCDR2
IYPGSVNT

SEQ ID NO: 121 - protein, 120 aa, synthetic construct
  REGION 1..120: note = Synthetic: aCD28 (VH_C55S, N62S, L111A), VH
QVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHYGADWNFDVWGQGTTVTVSS

SEQ ID NO: 122 - protein, 120 aa, synthetic construct
  REGION 1..120: note = Synthetic: aCD28 (VH_C55S, S36G, Y109F, L111Y), VH
QVQLVQSGAEVKKPGASVKVSCKASGYTFTGYYIHWVRQAPGQGLEWIGSIYPGNVNTNYNEKFKDRATLTVDTSISTAYMELSRLRSDDTAVYFCTRSHFGYDWNFDVWGQGTTVTVSS

SEQ ID NO: 123 - protein, 120 aa, synthetic construct
  REGION 1..120: note = Synthetic: aCD28 (VH_C55S, N62S, Y109F, L111Y), VH
QVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHFGYDWNFDVWGQGTTVTVSS

SEQ ID NO: 124 - protein, 13 aa, synthetic construct
  REGION 1..13: note = Synthetic: aCD28 (VH_C55S, L111Y, W113F), HCDR3
TRSHYGYDFNFDV

SEQ ID NO: 125 - protein, 120 aa, synthetic construct
  REGION 1..120: note = Synthetic: aCD28 (VH_C55S, L111Y, W113F), VH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSS

SEQ ID NO: 126 - protein, 13 aa, synthetic construct
  REGION 1..13: note = Synthetic: aCD28 (VH_C55S, W113A), HCDR3
TRSHYGLDANFDV

SEQ ID NO: 127 - protein, 120 aa, synthetic construct
  REGION 1..120: note = Synthetic: aCD28 (VH_C55S, W113A), VH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDANFDVWGQGTTVTVSS

SEQ ID NO: 128 - protein, 733 aa, synthetic construct
  REGION 1..733: note = Synthetic: 328M55 (chain 1)
QVQLVQSGAEVKKPGSSVKVSCKASGYTFTSYWMNWVRQAPGQCLEWMGMIHPSDSETRLNQKFKDRVTITADKSTSTAYMELSSLRSEDTAVYYCARQGIITSVQEFAYWGQGTLVTVSSGGGGSGGGGSGGGGSGGGGSIQMTQSPSSLSASVGDRVTITCSASSSVNYIYWYQQKPGKAPKLLIYRTSNLASGVPSRFSGSGSGTDFTLTISSLQPEDFATYYCQQYHSYPLTFGCGTKVEIKSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 129 - protein, 450 aa, synthetic construct
  REGION 1..450: note = Synthetic: 328M55 (chain 2) 328M56 (chain 2) 328M56s (chain 2) 328M58 (chain 2) 328M58s (chain 2) 328D19 (chain 2) 328C8 (chain 2) 328C9 (chain 2) 328D42 (chain 2)
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 130 - protein, 214 aa, synthetic construct
  REGION 1..214: note = Synthetic: 328M32 (chain 3) 328M55 (chain 3) 328M56 (chain 3) 328M56s (chain 3) 328M57 (chain 3) 328M58 (chain 3) 328M58s (chain 3) 328M59 (chain 3) 328M62 (chain 3) 328D18 (chain 3) 328D19 (chain 3) 328D20 (chain 3) 328D41 (chain 3) 328D42 (chain 3) 328D47 (chain 3) 328D55 (chain 3) 328C8 (chain 3) 328C9 (chain 3)
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 733 aa, synthetic construct
  REGION 1..733: note = Synthetic: 328M56 (chain 1) 328M57 (chain 1) 328M62 (chain 1)
QVQLVQSGAEVKKPGSSVKVSCKASGYTFTSYWMNWVRQAPGQCLEWMGMIHPSDSETRLNQKFKDRVTITADKSTSTAYMELSSLRSEDTAVYYCARQGIITSVQEFAYWGQGTLVTVSSGGGGSGGGGSGGGGSGGGGSIQMTQSPSSLSASVGDRVTITCSASSSVNYIYWYQQKPGKAPKLLIYRTSNLASGVPSRFSGSGSGTDFTLTISSLQPEDFATYYCQQYHSYPLTFGCGTKVEIKSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 132 - protein, 450 aa, synthetic construct
  REGION 1..450: note = Synthetic: 328M32 (chain 2) 328M57 (chain 2) 328M59 (chain 2) 328D20 (chain 2)
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDANFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 133 - protein, 733 aa, synthetic construct
  REGION 1..733: note = Synthetic: 328M58 (chain 1) 328M59 (chain 1)
QVQLVQSGAEVKKPGSSVKVSCKASGYTFTSYWMNWVRQAPGQCLEWMGMIHPSDSETRLNQKFKDRVTITADKSTSTAYMELSSLRSEDTAVYYCARQGIITSVQEFAYWGQGTLVTVSSGGGGSGGGGSGGGGSGGGGSIQMTQSPSSLSASVGDRVTITCSASSSVNYIYWYQQKPGKAPKLLIYRTSNLASGVPSRFSGSGSGTDFTLTISSLQPEDFATYYCQQYHSYPLTFGCGTKVEIKSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 134 - protein, 731 aa, synthetic construct
  REGION 1..731: note = Synthetic: 328D18 (chain 1) 328D19 (chain 1) 328D20 (chain 1) 328D47 (chain 1) 328D48 (chain 1) 328D49 (chain 1) 328D50 (chain 1)
QVQLVQSGAEVKKPGASVKVSCKASGYTFTDYFMNWVRQAPGQCLEWMGVINPYNDITIYNQKFQGRVTMTVDRSTSTVYMELSSLRSEDTAVYYCAREGVLYDGYYEGAYWGQGTLVTVSSGGGGSGGGGSGGGGSDIQLTQSPSFLSASVGDRVTITCKASQNVGIAVAWYQQKPGKAPKLLIYAASNRYTGVPSRFSGSGSGTEFTLTISSLQPEDFATYYCQQYSTYPYTFGCGTKLEIKSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 135 - protein, 450 aa, synthetic construct
  REGION 1..450: note = Synthetic: 328D18 (chain 2) 328D41 (chain 2)
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHFGYDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 136 - protein, 731 aa, synthetic construct
  REGION 1..731: note = Synthetic: 328D41 (chain 1) 328D42 (chain 1) 328D51 (chain 1) 328D52 (chain 1) 328D53 (chain 1) 328D54 (chain 1) 328D55 (chain 1)
QVQLVQSGAEVKKPGASVKVSCKASGYTFTDYFMNWVRQAPGQCLEWMGVINPYNDITIYNQKFQGRVTMTVDRSTSTVYMELSSLRSEDTAVYYCAREGVLYDGYYEGAYWGQGTLVTVSSGGGGSGGGGSGGGGSDIQLTQSPSFLSASVGDRVTITCKASQNVGIAVAWYQQKPGKAPKLLIYAASNRYTGVPSRFSGSGSGTEFTLTISSLQPEDFATYYCQQYSTYPYTFGCGTKLEIKSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 137 - protein, 733 aa, synthetic construct
  REGION 1..733: note = Synthetic: 328C8 (chain 1)
QVQLVQSGAEVKKPGASVKVSCKASGYTFTNSGMNWVRQAPGQCLEWMGWINTNTGEPTFAEEFRGRVTMTRDTSISTAYMELSRLRSDDTAVYYCARYYYGNSFAYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNNYFYPLTFGCGTKVEIKSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 138 - protein, 220 aa, synthetic construct
  REGION 1..220: note = Synthetic: 328C12 (chain 3) 328C13 (chain 3) 328C14 (chain 3) 328C15 (chain 3) 328C18 (chain 3) 328C19 (chain 3) 328C20 (chain 3)
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNNYFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 733 aa, synthetic construct
  REGION 1..733: note = Synthetic: 328C9 (chain 1)
QVQLVQSGAEVKKPGASVKVSCKASGYTFTNSGMNWVRQAPGQCLEWMGWINTNTGEPTFAEEFRGRVTMTRDTSISTAYMELSRLRSDDTAVYYCARYYYGNSFAYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNNYFYPLTFGCGTKVEIKSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 140 - protein, 739 aa, synthetic construct
  REGION 1..739: note = Synthetic: 328C12 (chain 1)
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 141 - protein, 448 aa, synthetic construct
  REGION 1..448: note = Synthetic: 328C12 (chain 2) 328C13 (chain 2) 328C20 (chain 2)
QVQLVQSGAEVKKPGASVKVSCKASGYTFTNSGMNWVRQAPGQGLEWMGWINTNTGEPTFAEEFRGRVTMTRDTSISTAYMELSRLRSDDTAVYYCARYYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 142 - protein, 739 aa, synthetic construct
  REGION 1..739: note = Synthetic: 328C13 (chain 1)
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 143 - protein, 481 aa, synthetic construct
  REGION 1..481: note = Synthetic: 328C14 (chain 1)
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 144 - protein, 704 aa, synthetic construct
  REGION 1..704: note = Synthetic: 328C14 (chain 2) 328C15 (chain 2)
QVQLVQSGAEVKKPGASVKVSCKASGYTFTNSGMNWVRQAPGQGLEWMGWINTNTGEPTFAEEFRGRVTMTRDTSISTAYMELSRLRSDDTAVYYCARYYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 145 - protein, 481 aa, synthetic construct
  REGION 1..481: note = Synthetic: 328C15 (chain 1) 328C19 (chain 1)
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 146 - protein, 5 aa, synthetic construct
  REGION 1..5: note = Synthetic: exemplary linker
GGGGS

SEQ ID NO: 147 - protein, 5 aa, synthetic construct
  REGION 1..5: note = Synthetic: exemplary linker
GKPGS

SEQ ID NO: 148 - protein, 5 aa, synthetic construct
  REGION 1..5: note = Synthetic: exemplary linker
GEPGS

SEQ ID NO: 149 - protein, 5 aa, synthetic construct
  REGION 1..5: note = Synthetic: exemplary linker
GGPGS

SEQ ID NO: 150 - protein, 733 aa, synthetic construct; identical to SEQ ID NO: 128
  REGION 1..733: note = Synthetic: 328M32 (chain 1)

SEQ ID NO: 151 - protein, 728 aa, synthetic construct
  REGION 1..728: note = Synthetic: 328M56s (Chain 1)
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 152 - protein, 728 aa, synthetic construct
  REGION 1..728: note = Synthetic: 328M58s (Chain 1)
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 153 - protein, 450 aa, synthetic construct
  REGION 1..450: note = Synthetic: 328M62 (chain 2) 328D47 (chain 2) 328D55 (chain 2)
QVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHFGYDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 154 - protein, 704 aa, synthetic construct
  REGION 1..704: note = Synthetic: 328D48 (Chain 2) 328D51 (Chain 2)
EVQLLESGGGLVQPGGSLRLSCAASGFTFSSYGMHWVRQAPGKGLEWVAVISHHGSSKYYADSVKGRFTISRDNSKNTLYLQMNSLRAEDTAVYYCARDWFFYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHFGYDWNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 155 - protein, 219 aa, synthetic construct
  REGION 1..219: note = Synthetic: 328D48 (Chain 3) 328D49 (Chain 3) 328D50 (Chain 3) 328D51 (Chain 3) 328D52 (Chain 3) 328D53 (Chain 3) 328D54 (Chain 3)
DIQMTQSPSSLSASVGDRVTITCKSSQSLLHSDAKTFLYWYQQKPGKAPKLLIYEVSNRFSGVPSRFSGSGSGTDFTLTISSLQPEDFATYYCLQGERLPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 704 aa, synthetic construct
  REGION 1..704: note = Synthetic: 328D49 (Chain 2) 328D52 (Chain 2)
EVQLLESGGGLVQPGGSLRLSCAASGFTFSSYGMHWVRQAPGKGLEWVAVISHHGSSKYYADSVKGRFTISRDNSKNTLYLQMNSLRAEDTAVYYCARDWFFYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHFGYDWNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 157 - protein, 704 aa, synthetic construct
  REGION 1..704: note = Synthetic: 328D50 (Chain 2) 328D53 (Chain 2)
EVQLLESGGGLVQPGGSLRLSCAASGFTFSSYGMHWVRQAPGKGLEWVAVISHHGSSKYYADSVKGRFTISRDNSKNTLYLQMNSLRAEDTAVYYCARDWFFYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGYDFNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 158 - protein, 704 aa, synthetic construct
  REGION 1..704: note = Synthetic: 328D54 (Chain 2)
EVQLLESGGGLVQPGGSLRLSCAASGFTFSSYGMHWVRQAPGKGLEWVAVISHHGSSKYYADSVKGRFTISRDNSKNTLYLQMNSLRAEDTAVYYCARDWFFYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDANFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 159 - protein, 481 aa, synthetic construct
  REGION 1..481: note = Synthetic: 328C18 (chain 1)
EVQLVESGGGLVQPGGSLRLSCAASGFTFSTYAMNWVRQAPGKGLEWVGRIRSEANNYATYYADSVKGRFTISRDDSKNTLYLQMNSLRAEDTAVYYCVRHGNFGDSYVSWFEYWGQGTLVTVSSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 160 - protein, 704 aa, synthetic construct
  REGION 1..704: note = Synthetic: 328C18 (chain 2) 328C19 (chain 2)
QVQLVQSGAEVKKPGASVKVSCKASGYTFTNSGMNWVRQAPGQGLEWMGWINTNTGEPTFAEEFRGRVTMTRDTSISTAYMELSRLRSDDTAVYYCARYYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGGGGGSGGGGSQVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHFGYDWNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 161 - protein, 739 aa, synthetic construct
  REGION 1..739: note = Synthetic: 328C20 (chain 1)
QVQLVQSGAEVKKPGASVKVSCKASGYTFTSYYIHWVRQAPGQGLEWIGSIYPGSVNTNYNEKFKDRATLTVDTSISTAYMELSRLRSDDTAVYFCTRSHFGYDWNFDVWGQGTTVTVSSGGGGSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFEYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

(19) United States
(12) Patent Application Publication
(10) Pub. No.: US 2025/0388703 A1
(43) Pub. Date: Dec. 25, 2025

(54) MULTI-SPECIFIC MOLECULES AND MENTOD OF USE AND MAKING THEREOF

(71) Applicant: GENSUN BIOPHARMA INC., Newbury Park, CA (US)

(72) Inventors: Jackie SHENG, Newbury Park, CA (US); Shude YAN, Newbury Park, CA (US); Tyler LIBAN, Newbury Park, CA (US)

(21) Appl. No.: 18/748,953

(22) Filed: Jun. 20, 2024

(51) Int. Cl. C07K 16/46; A61K 39/00; A61P 35/00; A61P 37/04

(52) U.S. Cl. CPC C07K 16/468; A61P 35/00; A61P 37/04; A61K 2039/505; C07K 2317/31; C07K 2317/52; C07K 2317/565; C07K 2317/92

(57) ABSTRACT

Provided are multi-specific molecules that can simultaneously engage T cells and target cancer or tumor cells. Also provided are methods of treating a cell proliferative disorder such as a cancer or tumor using the multi-specific molecules. In certain embodiments, a multi-specific molecule, comprises a first targeting domain that specifically binds to human CD3; a second targeting domain that specifically binds to human CD28; a third targeting domain that specifically binds to human Muc17, or human DLL3, or human CLDN18.2; and an Fc fragment that lacks antibody-dependent cellular cytotoxicity.

### FIELD OF INVENTION

This invention relates generally to cancer therapies, and more specifically, to novel molecules comprising one or more T cell engaging domains and one or more cancer- or tumor-targeting domains.

### BACKGROUND

Conventional cancer treatments are directed at removing cancerous tissue and preventing it from spreading. Such treatment options include surgery, chemotherapy, radiation therapy, hormonal therapy, targeted therapy and palliative care. Treatments are usually pursued based on the type, location and grade of the cancer as well as the patient's health and preferences. These options have limitations. They can be ineffective, particularly when cancer has metastasized. Moreover, chemotherapy and radiation therapy have a range of side-effects related to cell toxicity. Accordingly, more effective agents and methods with reduced side-effects are still needed to for the treatment of cancer.

### SUMMARY

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

One aspect of the present application relates to a multi-specific protein molecule that comprises (a) a first targeting domain that specifically binds to human CD3 with a binding affinity that equals to, or is greater than, 1 nmol/L, (b) a second targeting domain that specifically binds to human CD28 with a binding affinity that equals to, or is greater than, 100 nmol/L; and (c) a third targeting domain that specifically binds to human Muc17, human DLL3, or human CLDN18.2.

Another aspect of the present application relates to polynucleotide encoding a multi-specific protein molecule of the present application.

Another aspect of the present application relates to an expression vector capable of expressing a multi-specific protein molecule of the present application in a target cell harboring the expression vector.

Another aspect of the present application relates to a method of activating T cells in a subject with a multi-specific protein molecule of the present application or an expression vector of the present application.

Another aspect of the present application relates to a method of treating cancer or tumor in a subject with a multi-specific protein molecule of the present application or an expression vector of the present application.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 depicts exemplary configurations of multi-specific molecules of the present application with CD3-, CD28- and Muc17-targeting domains (328M TriTEs, Panels A-H).

FIG. 2 shows benchmark (BM) molecules (Panels A and B), exemplary 328M configuration (Panel C) and target binding affinities and activities of embodiments of 328M TriTEs (Panel D).

FIG. 3 shows results of cytokine release assays that assess nonspecific cytokine secretion from PBMC cells exposed to multi-specific molecules of the present application. Panels A and D, release of IFNγ. Panels B and E, release of IL-6. Panels C and F, release of TNFα. OKT3: monoclonal anti-Human CD3 antibody (BioLegend, Cat #317326). 328BM: SAR443216

FIG. 4 shows example results of T Cell Dependent Cellular Cytotoxicity (TDCC) assays. Panels A and C, high ratio of PBMC to ASPC1 (5 Effector PBMCs:1 ASPC1 cancer cell). Panels B and D, low ratio of PBMC to ASPC1 (1 PBMC:10 ASPC1 cancer cells).

FIG. 5 shows that multi-specific molecules with CD3-, CD28- and Muc17-targeting domains stimulate IL-2 secretion and T cell activation/proliferation upon target engagement. Panel A, secretion of IL-2 from target cells. Panel B, cell count of CD4+ CD25+ T cells after treatment. Panel C, Cell counts of CD8+CD25+ T cells after treatment.

FIG. 6 shows the results of in vivo study with multi-specific molecules with CD3-, CD28- and Muc17-targeting domains. Significance was determined by T.TEST analysis. n.s.: not significant, *: p<0.05.

FIG. 7 shows that the second generation 328M TriTEs induce minimal IL-6 secretion in cynomolgus monkeys compared to first-generation TriTE (328M5) and match clinical BM BITE. Panel A, IL-6 secretion induced by 328M56s. Panel B, IL-6 secretion induced by BM BiTE and 328M58s. Panel C, IL-6 secretion induced by first generation TriTE 328M5.

FIG. 8 shows that second-generation TriTEs do not cause body weight loss in cynomolgus monkey as compared to first-generation TriTE (328M5) and match clinical BM BITE. No obvious body weight change for 328M56s up to 9 mg/kg and no obvious abnormal clinical observations (Panels B and C). In contrast, significant body weight loss was observed for a first generation TriTE, 328M5, targeting the same TAA, even at 1 mg/kg (Panel A), along with other toxicities.

FIG. 9, Panels A-I, depict embodiments of the multi-specific molecules of the present application with CD3-, CD28- and DLL3-targeting domains (328D TriTE molecules).

FIG. 10 summarizes target binding affinities and non-specific cytokine release of embodiments of 328D TriTE molecules.

FIG. 11 shows an example of results of cytokine release assays that assess nonspecific cytokine secretion from PBMC cells exposed to several 328D TriTE example molecules. Panel A and B, release of TNFα. Panel C and D, release of IL-6.

FIG. 12 shows the example of screening TriTEs in TDCC assays using various cell lines on second generation 328D TriTE molecules. Panel A, huDLL3⁺ CHO (constructed in house) TDCC using E:T=1:10 Panel B, and C, NCI-H82 cells (purchased from ATCC), using E:T=1:5, Panel D, NCI-H2286 cells (purchased from ATCC), using E:T=1:6.

FIG. 13 shows comparative analysis of second generation 328D TriTE molecules, benchmark BITE (BM BITE) of similar weak CD3 Affinity, and combination treatment in TDCC assay in cancer cell line NCI-H82 (Panel A) and SHP77 (Panel B). Effector PBMCs:target cell ratio=1:5. NCI-H82 and SHP77 were purchased from ATCC.

FIG. 14 shows efficacy of 328D molecules on tumor growth in huCD3/huCD28 mice. Panel A: Effect of second generation 328D in huCD3/huCD28 mice bearing B16F10-hDLL3 cells. Panel B: Effect of second generation 328D TriTE in NOG mice bearing PBMC mixed with SHP-77 cells.

FIG. 15, Panels A-I, depict embodiments of the multi-specific molecules of the present application with CD3-, CD28- and CLDN18-targeting domains (328C series second generation TriTE molecules).

FIG. 16 depicts embodiments of 328C TriTE molecules (Panels A-C) and designed target binding affinities and non-specific cytokine release of the TriTE molecules (Panels D).

FIG. 17 shows an example of results of cytokine release assays that assess nonspecific cytokine secretion from cells exposed to 328C TriTE molecules. Panel A, release of IFNγ. Panel B, release of IL2. Panel C, release of IL6. Panel D, release of TNFα.

FIG. 18 shows an example result of TDCC assays that demonstrate second generation 328C TriTEs' advantage over corresponding BiTE molecule (3CBM). Panel A, CHO-CLDN⁺ TDCC at high ratio of PBMC to CHO (E:T=10:1). Panel B, CHO-CLDN killing at low ratio of PBMC to CHO Cldn⁺ (E:T=1:10). Panel C, EC50 of example TriTEs in CHO-CLDN⁺ killing at high and low ratios of PBMC to target cells.

FIG. 19 shows example results of TDCC assays that compare different TriTEs at different effortor:target (E:T) ratios. Panel A, GSU killing at E:T=3:1. Panel B, GSU killing at E:T=1:9. Panel C: EC50 of GSU killing at different E:T ratios. GSU cell was purchased from Creative Bioarray.

### DETAILED DESCRIPTION

### I. Definitions

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In an embodiment, a “subject” of diagnosis or treatment is a prokaryotic or a eukaryotic cell, a tissue culture, a tissue or an animal, e.g. a mammal, including a human.

The term “multi-specific” refers to a molecule (such as an antibody molecule) comprising at least two targeting domains. Each targeting domain is capable of binding specifically to a target molecule or a target epitope. In some embodiments, the multi-specific molecule is a polymeric molecule having two or more peptides. In some embodiments, the targeting domain comprises one or more antigen binding domains, or one or more CDRs of an antibody.

The term “bi-specific T cell engager” or “BiTE”) refers to a molecule (such as an antibody) having two targeting domains that specifically bind to two different target molecules or epitopes. Each targeting domain is capable of binding specifically to a target molecule or a target epitope. In some embodiments, the bi-specific molecule is a polymeric molecule having two or more peptides. In some embodiments, the targeting domain comprises one or more antigen binding domains, or one or more CDRs of an antibody. Examples of bi-specific T cell engagers include, but are not limited to, bi-specific antibodies with a targeting domain that binds specifically to CD3 and a targeting domain that binds specifically to a Tumor Associated Antigen (TAA).

The term “tri-specific T cell engager” or “TriTE”) refers to a molecule (such as an antibody) having three targeting domains that specifically bind to three different target molecules or epitopes. Each targeting domain is capable of binding specifically to a target molecule or a target epitope. In some embodiments, the tri-specific molecule is a polymeric molecule having two or more peptides. In some embodiments, the targeting domain comprises one or more antigen binding domains, or one or more CDRs of an antibody. Examples of tri-specific T cell engagers include, but are not limited to, tri-specific antibodies with (1) a targeting domain that binds specifically to CD3, (2) a targeting domain that binds specifically to CD28, and (3) a targeting domain that binds specifically to Muc17, DLL3 or CLDN18.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “pharmaceutical composition” is intended to include the combination of an active agent, such as a multi-specific molecule of the present application, with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

The term “effective amount” refers, without limitation, to the amount of the defined component sufficient to achieve the desired therapeutic result. In an embodiment, that result can be effective cancer treatment.

The terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

As used herein, the term “recombinant” refers to polypeptides or polynucleotides that do not exist naturally and which may be created by combining polynucleotides or polypeptides in arrangements that would not normally occur together.

As used herein, the term “antibody” refers to a polypeptide or a polypeptide complex that specifically recognizes and binds to an antigen through one or more immunoglobulin variable regions. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding and is encoded by the variable domain.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain VL-CL joined to VH-CH1 by a disulfide bond. While various antibody fragments are defined in terms of the digestion of an intact antibody, one of skill will appreciate that such fragments may be synthesized de novo either chemically or by using recombinant DNA methodology. Thus, the term antibody, as used herein, includes a whole antibody, an antigen binding fragment or a single chain thereof. The term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term antibody also embraces minibodies, scFvs, diabodies, triabodies and the like. ScFvs and Diabodies are small bivalent biospecific antibody fragments with high avidity and specificity. Their high signal to noise ratio is typically better due to a better specificity and fast blood clearance increasing their potential for diagnostic and therapeutic targeting of specific antigen (Sundaresan et al., J Nucl Med 44:1962-9 (2003). In addition, these antibodies are advantageous because they can be engineered if necessary as different types of antibody fragments ranging from a small single chain Fv (scFv) to an intact IgG with varying isoforms (Wu & Senter, Nat. Biotechnol. 23:1137-1146 (2005)). In some embodiments, the antibody fragment is part of a scFv-scFv or diabody. In some embodiments, in either aspect, the invention provides high avidity antibodies for use according to the invention.

The term “antigen-binding fragment” or “Fab” refers to a region on an antibody that binds to antigens. It includes one constant and one variable domain of each of the heavy and the light chains (i.e. four domains: VH, CH1, VL and CL1). The variable domain contains the paratope (the antigen-binding site), that includes a set of complementary determining regions (CDRs) at the amino terminal end of the monomer. Each arm of the Y thus binds an epitope on the antigen. The CDR sequences listed in the present application are based on the definition of the International ImMunoGeneTics Information System® (IMGT®).

The term “Fc region” or “fragment crystallizable region” refers to the tail region of an antibody CH2-CH3 that interacts with cell surface receptors called Fc receptors and some proteins of the complement system.

In IgG, IgA and IgD antibody isotypes, the Fc region has two identical protein fragments, derived from the second and third constant domains of the antibody's two heavy chains. IgM and IgE Fc regions have three heavy chain constant domains (CH domains 2-4) in each polypeptide chain whereas IgG is composed of 2 CH domains, 2 and 3. The Fc regions of IgGs bear a highly conserved N-glycosylation site. Glycosylation of the Fc fragment is essential for Fc receptor-mediated activity. The N-glycans attached to this site are predominantly core-fucosylated diantennary (?) structures of the complex type. In addition, small amounts of these N-glycans also bear bisecting GlcNAc and α-2, 6 linked sialic acid residues.

A particular IgG subclass can be preferred for a particular use. For example, IgG1 is more effective than IgG2 and IgG4 at mediating ADCC and CDC. Thus, IgG2 Fc can be preferred when effector function is undesirable. However, IgG2 Fc-containing molecules are generally more difficult to manufacture and can be less stable than IgG1 Fc-containing molecules.

The effector function of an antibody can be increased, or decreased, by introducing one or more mutations into the Fc (see, for example, Strohl, Curr. Opin. Biotech., 20:685-691, 2009).

The term “silent Fc” or “silent Fc fragment”-refers to modified Fc fragment that is deficient in Fc-mediated immune effector functions allow antibodies to activate the immune system leading to antibody-dependent cellular cytotoxicity (ADCC), antibody-dependent cellular phagocytosis (ADCP), and/or complement dependent cytotoxicity (CDC). ADCC and ADCP are mediated through the binding of the Fc to Fc receptors on the surface of cells of the immune system. CDC is mediated through the binding of the Fc with proteins of the complement system, (e.g. C1q). The Fc-mediated immune effector functions are an important part of an antibody's natural function, but in many therapeutic antibodies, these interactions are not desirable and can lead to catastrophic side effects. The “silent Fc” or “silent Fc fragment” of the present application has decreased or abolished immune effector function. Examples of silent Fc include, but are not limited to, those having the following substitutions: L234A/L235A/P329G (LALAPG, IgG1), N297A or N297Q (IgG1), L234A/L235A (IgG1), C220S/C226S/C229S/P238S (IgG1), C226S/C229S/E233P/L234V/L235A (IgG1), L234F/L235E/P331S (IgG1), S267E/L328F (IgG1), V234A/G237A (IgG2), L235A/G237A/E318A (IgG4), H268Q/V309L/A330S/A331S (IgG2), L234A/L235A/G237A (IgG1), L234A/L235A/G237A/P238S/H268A/A330S/P330S, L234A/L235E (IgG1), G236R/L328R (IgG1), or L234A/L235A/K322A (IgG1). Approaches to eliminate Fc-mediated immune effector function are well known in the art.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CHI, CH2 and CH3) confer important biological properties such as secretion, transplacental mobility, Fc receptor binding, complement binding, and the like. By convention, the numbering of the constant region domains in conventional antibodies increases as they become more distal from the antigen-binding site or amino-terminus of the antibody. In conventional antibodies, the N-terminal portion is a variable region and at the C-terminal portion is a constant region; the CH3 and CL domains comprise the carboxyterminus of the heavy and light chain, respectively.

As used herein, the term “heavy chain constant region” includes amino acid sequences derived from an immunoglobulin heavy chain. A polypeptide comprising a heavy chain constant region comprises at least one of: a CHI domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CHI domain; a polypeptide chain comprising a CHI domain, at least a portion of a hinge domain, and a CH2 domain; a polypeptide chain comprising a CHI domain and a CH3 domain; a polypeptide chain comprising a CHI domain, at least a portion of a hinge domain, and a CH3 domain, or a polypeptide chain comprising a CHI domain, at least a portion of a hinge domain, a CH2 domain, and a CH3 domain. In some embodiments, a polypeptide of the disclosure comprises a polypeptide chain comprising a CH3 domain. Further, an antibody for use in the disclosure may lack at least a portion of a CH2 domain (e.g., all or part of a CH2 domain). It should be understood that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CHI domain derived from an IgG1 molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain. A “light chain heavy chain pair” refers to the collection of a light chain and heavy chain that can form a dimer through a disulfide bond between the CL domain of the light chain and the CHI domain of the heavy chain.

The subunit structures and three-dimensional configurations of the constant regions of the various immunoglobulin classes are well known. As used herein, the term “VH domain” includes the amino terminal variable domain of an immunoglobulin heavy chain and the term “CHI domain” includes the first (most amino terminal) constant region domain of an immunoglobulin heavy chain. The CHI domain is adjacent to the VH domain and is amino terminal to the hinge region of an immunoglobulin heavy chain molecule.

The term “scFv” or “scFv fragment antibody” refers to a small molecular antibody, consisting of VH and VL domains, either in the configuration of VL-VH or VH-VL, with a linker region between them. The scFv fragment antibody can more easily penetrate blood vessel wall and the solid tumor, which makes it a preferred carrier of targeting drugs.

The term “scFvs” or “single-chain variable fragment” refers to divalent (or bivalent) single-chain variable fragments (di-scFvs, bi-scFvs) that can be engineered by linking two scFvs. This can be done by producing a single peptide chain with two VH and two VL regions, yielding tandem scFvs, also known as scFv-scFv molecules. Another possibility is the creation of scFvs with linker peptides that are too short for the two variable regions to fold together (about five amino acids), forcing scFvs to dimerize. This type is known as diabodies.

The term “humanized antibody” refers to an antibody from non-human species whose protein sequences have been modified to increase its similarity to antibody variants produced naturally in humans. The process of “humanization” is usually applied to monoclonal antibodies developed for administration to humans (e.g. antibodies developed as anti-cancer drugs). Humanization can be necessary when the process of developing a specific antibody involves generation in a non-human immune system (such as that in mice).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

One of the challenges for efficiently producing multispecific antibody preparations concerns reducing the formation of homodimeric molecules in favor of heterodimeric molecules, when co-expressing chains of different binding specificities. A “heterodimeric antibody” can utilize the “knobs-into-holes” or “charge-pair” formats to preferentially promote correct association of the 2 molecules to form a heterodimer with 2 specificities. These formats are specific to the heavy chain Fc part of the constant region in antibodies. For the knob-into-holes format, the “knobs” part is engineered by replacing a small amino acid with a larger one. It fits into the “hole,” which is engineered by replacing a large amino acid with a smaller one. Introduction of T366W mutations in the first Fc creates the “knob” and introduction of T366S, L368A, and Y407V mutations in the second Fc creates the “hole” (numbering of the residues according to the Kabat EU numbering system). For the charge pair format, heterodimerization is favored through stabilizing ionic interactions by introducing interfacing charge residues in the opposing Fc domains. For example, D356K, E357K, and D399K in a first Fc domain, and the mutations K370E, K409D, and K439E into a second Fc domain, or combination thereof. For example, K392D and K409D mutations in a first Fc chain, and D399K and D356K mutations in a second Fc chain, K409E in the first Fc and D399K in the Fc, K409E in the first Fc and D399R in the second Fc, K409D in the first Fc and D399K in the second Fc, K409D in the first Fc and D399R in the second Fc, K392E in the first Fc and D399R in the second Fc, K392E in the first Fc and D399K in the second Fc, K392D in the first Fc and D399R in the second Fc, K392D in the first Fc and D399K in the second Fc, K409D and K360D in the first Fc and D399K and D356K in the second Fc, K409D and K370D in the first Fc and D399K and E357K in the second Fc, K409D and K392D in the first Fc and D399K, D356K, and E357K in the second Fc, K409D and K392D in the first Fc and D399K in the second Fc, K409D and K392D in the first Fc and D399K and D356K in the second Fc, K409D and K392D in the first Fc and D399K and E357K in the second Fc, K409D and K370D in the first Fc and D399K and D357K in the second Fc, D399K in the first Fc and K409D and K360D in the second Fc, and/or K409D and K439D in the first Fc and D399K and D356K in the second Fc, numbered according to the Kabat EU numbering system. Additionally, cysteines may be introduced to stabilize the pairing of heterodimers, for example S234C in the first Fc and Y349C in the second Fc or Y349C in the first Fc and S344C in the second Fc.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity, neurodegeneration or pathological inflammation, normal human cells or tissues.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273 (52): 35095-35101 (1998).

As used herein, the term “treatment” means all of the actions by which the symptoms of the disease have been alleviated, improved or ameliorated. In the present specification, “treatment” means that the symptoms of cancer, neurodegeneration, or infectious disease are alleviated, improved or ameliorated by administration of the antibodies disclosed herein.

Construction of suitable vectors containing the desired sequences and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

The term “cancer” refers to human cancers and carcinomas, sarcomas, adenocarcinomas, etc., including solid tumors, kidney, breast, lung, kidney, bladder, urinary tract, urethra, penis, vulva, vagina, cervical, colon, ovarian, prostate, pancreas, stomach, brain, head and neck, skin, uterine, testicular, esophagus, and liver cancer. Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

As the patients and subjects of the invention method are, in addition to humans, veterinary subjects, formulations suitable for these subjects are also appropriate. Such subjects include livestock and pets as well as sports animals such as horses, greyhounds, and the like.

### II. Multi-Specific Molecules of the Present Application

One aspect of the present application relates to multi-specific molecules that comprises: a) one or more T cell engaging domains that specifically binds, with a medium or low binding affinity, to one or more receptors on T cell surface; and b) one or more cancer/tumor cell engaging domains that specifically bind to one or more target molecules expressed on the surface of target cancer/tumor cells. In contrast to previously disclosed multi-specific molecules that contain one or more T cell engaging domains that specifically binds, with a high binding affinity, to one or more receptors on T cell surface (also referred to as “first-generation” multi-specific molecules), the multi-specific molecules of the present application (also referred to as “second generation” multi-specific molecules) are capable of engaging both T cells and target cancer cell to facility T cell-mediated killing of the target cancer cells. The T-cell targeting domains with reduced binding affinities allow the second generation multi-specific molecules to have reduced cytotoxicity (e.g., toxicity associated with nonspecific cytokine releases syndrome (CRS)), while maintaining their T-cell activation potency and their anti-proliferation potency for the target cancer cells.

In some embodiments, the multi-specific molecules of the present application further comprises a silent Fc fragment. In some embodiments, the multi-specific molecules of the present application are humanized antibody molecules.

### T Cell Engaging Domains

T cell engaging domains allow the multi-specific molecules of the present application to bind specifically to T cells and activate T cell-mediated cytotoxicity. Examples of T cell-engaging domains include, but are not limited to, CD3-targeting domains and CD28 targeting domains. CD3 and CD28 are receptors present on T-cells. T cells can be activated though CD3 and CD28, by antigen-presenting cells that utilize the activation signals MHC Class I and II, CD80 and CD86, and 4-1BBL, respectively. CD3 is part of the T cell receptor (TCR) and is the signaling component for the receptor. There are three CD3 subunits, epsilon, delta and gamma. Epsilon associates with both delta and zeta and together they are responsible for signaling. CD3 signaling is considered signal 1 that is required to activate T cells. The co-receptors, such as CD28 and CD137, are considered signal 2. Both signal 1 and signal 2 are required for full activation, proliferation and survival of T cells. The inventors have unexpectedly discovered that multi-specific molecules that comprise CD3 and/or CD28 targeting domains with medium or low binding affinity to CD3/CD28 have significantly reduced CRS toxicity, without significant loss of their T cell activation and/or anti-cancer potency.

In some embodiments, the multi-specific molecules of the present application comprise one or more CD3-targeting domains having low or medium binding affinity to CD3, preferably human CD3. In some embodiments, the CD3-targeting domain derives from a parental anti-CD3 monoclonal antibody.

As used herein, the term “medium binding affinity” for CD3 is a binding affinity within the range of 1-200 nmol/L. As used herein, the term “low binding affinity” for CD3 typically refers to a binding affinity within the range of 200-2000 nmol/L. In some embodiments, the binding affinity refers to the binding affinity between a multi-specific molecule with a CD3-targeting domain and CD3+ cells. In some embodiments, the binding affinity refers to the affinity between a monoclonal antibody and CD3+ cells. In some embodiments, the binding affinity is determined by the method described in Example 1.

In some embodiments, the multi-specific molecules of the present application comprise one or more CD28-targeting domains having low or medium binding affinity to CD28, preferably human CD28. In some embodiments, the CD28-targeting domain derives from a parental anti-CD28 monoclonal antibody.

As used herein, the term “medium binding affinity” for CD28 is a binding affinity within the range of 10-500 nmol/L. As used herein, the term “low binding affinity” for CD28 is a binding affinity within the range of 500-5000 nmol/L. In some embodiments, the binding affinity refers to the binding affinity between a multi-specific molecule with a CD28-targeting domain and CD28+ cells. In some embodiments, the binding affinity refers to the affinity between a monoclonal antibody and CD28+ cells. In some embodiments, the binding affinity is determined by the method described in Example 1 of the present application.

The T cell-targeting domains, such as the CD3 and/or CD28 targeting domains, in the multi-specific molecule of the present application can be a fragment (such as a Fab fragment) or in scFv form of an antibody.

In some embodiments, the anti-CD3 and/or anti-CD28 antibodies with medium or low binding affinities are generated by the methods described in Example 2 of the present application.

Examples of anti-human CD3 antibodies with medium and low binding affinity and Examples of anti-human CD28 antibodies with medium and low binding affinity are listed below in Table 1.

In some embodiments, the multi-specific molecules of the present application comprise a targeting domain that specifically binds to human CD3 with a medium or low binding affinity is in the form of a Fab fragment or a scFv. In some embodiments, the targeting domain that specifically binds to human CD3 with a medium or low binding affinity comprise (1) a heavy chain CDR1 comprising SEQ ID NO:93, (2) a heavy chain CDR2 comprising a sequence selected from the group consisting of SEQ ID NOS: 94, 101 and 103, (3) a heavy chain CDR3 comprising a sequence selected from the group consisting of SEQ ID NOS: 95 and 105, (4) a light chain CDR1 comprising the sequence of SEQ ID NO:96, (5) a light chain CDR2 comprising the sequence of SEQ ID NO:97, and (6) a light chain CDR3 comprising SEQ ID NO:98. In some embodiments, the targeting domain that specifically binds to human CD3 with a medium or low binding affinity comprise (1) a heavy chain variable region sequence selected from the group consisting of SEQ ID NOS: 102, 104 and 106; and/or (2) a light chain variable region sequence selected from the group consisting of SEQ ID NOS: 100.

In some embodiments, the multi-specific molecules of the present application comprise a targeting domain that specifically binds to human CD28 with a medium or low binding affinity is in the form of a Fab fragment or a scFv. In some embodiments, the targeting domain that specifically binds to human CD28 with a medium or low binding affinity comprise (1) a heavy chain CDR1 comprising a sequence selected from the group consisting of SEQ ID NOS: 107 and 117 (2) a heavy chain CDR2 comprising a sequence selected from the group consisting of SEQ ID NOS: 108 and 120, (3) a heavy chain CDR3 comprising a sequence selected from the group consisting of SEQ ID NOS: 109, 115, 118, 124 and 126, (4) a light chain CDR1 comprising the sequence of SEQ ID NO: 110, (5) a light chain CDR2 comprising the sequence of SEQ ID NO: 111, and (6) a light chain CDR3 comprising SEQ ID NO: 112. In some embodiments, the targeting domain that specifically binds to human CD28 with a medium or low binding affinity comprise (1) a heavy chain variable region sequence selected from the group consisting of 116, 119, 121, 122, 123, 125, and 127; and/or (2) a light chain variable region sequence selected from the group consisting of 114.

### Cancer/Tumor Cell Engaging Domains

The multi-specific molecules of the present application comprise one or more cancer cell engaging domains that allow the multi-specific molecule to engage both T cells and the target cancer cells simultaneously to facilitate T cell-mediated killing of the target cancer cells. Examples of cancer cell engaging domains include, but are not limited to, Muc17-targeting domains, DLL3-targeting domains, and CLDN18.2 targeting domains.

Mucin 17, also referred to as MUC17, is a member of the mucin family that is composed of more than 20 members. MUC17 is expressed in pancreatic, appendiceal, and some colon cancers and thus is a target antigen for these cancers.

Delta-like ligand 3 (DLL3) is an inhibitory notch ligand that is expressed at high levels in small cell lung cancer (SCLC) and other neuroendocrine tumors.

Claudin-18 (CLDN18) is a protein in humans that is encoded by the CLDN18 gene. It belongs to the group of claudins, a family of proteins that form components of tight cell junction strands in epithelial cells. Isoform 2 of CLDN (Claudin 18.2 or CLDN18.2) is abundant in gastric tumors. It has exposed extracellular loops and is available for monoclonal antibody binding.

Examples of anti-human Muc17 antibodies, anti-human DLL3 antibodies, and anti-human CLDN18.2 antibodies are listed in Table 1.

TABLE 1
Exemplary CDRs, VH and VL sequences

| Antibody | | HCDR1 | HCDR2 | HCDR3 | LCDR1 | LCDR2 | LCDR3 | VH | LH |
|---|---|---|---|---|---|---|---|---|---|
| domain | Clone Name | Seq ID | Seq ID | Seq ID | Seq ID | Seq ID | Seq ID | Seq ID | Seq ID |
| anti-Muc17 | hu1MU32A | 1 | 2 | 3 | 4 | 5 | 6 | 7 | 8 |
| anti-Muc17 | hu1MU11A | 9 | 10 | 11 | 12 | 5 | 13 | 14 | 15 |
| anti-Muc17 | hu1MU8A | 1 | 16 | 3 | 4 | 5 | 6 | 17 | 18 |
| anti-Muc17 | hu1MU37 | 19 | 20 | 21 | 22 | 23 | 24 | 25 | 26 |
| anti-Muc17 | HuS_anti-Muc17 | 27 | 28 | 29 | 30 | 31 | 32 | 33 | 34 |
| anti-DLL3 | huD139A | 35 | 36 | 37 | 38 | 39 | 40 | 41 | 42 |
| anti-DLL3 | hu1D4 | 43 | 44 | 45 | 46 | 47 | 48 | 49 | 50 |
| anti-DLL3 | huD143 | 51 | 52 | 53 | 54 | 55 | 56 | 57 | 58 |
| anti-DLL3 | huD138C | 59 | 60 | 53 | 54 | 61 | 62 | 63 | 64 |
| anti-CLDN | huCldn29B | 65 | 66 | 67 | 68 | 69 | 70 | 71 | 72 |
| anti-CLDN | huCldn16A | 73 | 74 | 75 | 76 | 69 | 77 | 78 | 79 |
| anti-CLDN | huCldn20 | 80 | 81 | 82 | 68 | 69 | 83 | 84 | 85 |
| anti-CLDN | huCldn23A | 86 | 87 | 88 | 89 | 69 | 90 | 91 | 92 |
| anti-CD3_3 | aCD3 (VH_A116E) | 93 | 94 | 95 | 96 | 97 | 98 | 99 | 100 |
| anti-CD3_20 | aCD3 (VH_A116E, | 93 | 101 | 95 | 96 | 97 | 98 | 102 | 100 |
| | Y60A) | | | | | | | | |
| anti-CD3_70 | aCD3 (VH_A116E, | 93 | 103 | 95 | 96 | 97 | 98 | 104 | 100 |
| | K59E, Y60A) | | | | | | | | |
| anti-CD3_400 | aCD3 (VH_A116D) | 93 | 94 | 105 | 96 | 97 | 98 | 106 | 100 |
| anti-CD28_wt | aCD28 (VH_C55S) | 107 | 108 | 109 | 110 | 111 | 112 | 113 | 114 |
| anti-CD28_100 | aCD28 (VH_C55S, | 107 | 108 | 115 | 110 | 111 | 112 | 116 | 114 |
| | Y109F, L111Y) | | | | | | | | |
| anti-CD28_300a | aCD28 (VH_C55S, | 117 | 108 | 118 | 110 | 111 | 112 | 119 | 114 |
| | S36G, L111A) | | | | | | | | |
| anti-CD28_300b | aCD28 (VH_C55S, | 107 | 120 | 118 | 110 | 111 | 112 | 121 | 114 |
| | N62S, L111A) | | | | | | | | |
| anti-CD28_300c | aCD28 (VH_C55S, | 117 | 108 | 115 | 110 | 111 | 112 | 122 | 114 |
| | S36G, Y109F, | | | | | | | | |
| | L111Y) | | | | | | | | |
| anti-CD28_300d | aCD28 (VH_C55S, | 107 | 120 | 115 | 110 | 111 | 112 | 123 | 114 |
| | N62S, Y109F, | | | | | | | | |
| | L111Y) | | | | | | | | |
| anti-CD28_700 | aCD28 (VH_C55S, | 107 | 108 | 124 | 110 | 111 | 112 | 125 | 114 |
| | L111Y, W113F) | | | | | | | | |
| anti-CD28_850 | aCD28 (VH_C55S, | 107 | 108 | 126 | 110 | 111 | 112 | 127 | 114 |
| | W113A) | | | | | | | | |

### Exemplary Multi-Specific Molecules

In some embodiments, the multi-specific molecules that comprises: a) a first targeting domain that specifically binds to human CD3 with a medium or low binding affinity; b) a second targeting domain that specifically binds to human CD28 with medium or low binding affinity; and c) a third targeting domains that specifically binds to targets selected from the group consisting of Muc17, DLL3, and CLDN18.2. The multi-specific molecules of the present application are capable of engaging both T cells and target cancer cell to facility T cell-mediated killing of the target cancer cells. The CD3/CD28 target domains with reduced binding affinities allow the multi-specific molecules of the present application to have reduced toxicity associated with nonspecific cytokine releases syndrome (CRS), while maintaining their T-cell activation potency and their anti-proliferation potency for the target cancer cells. In some embodiments, the multi-specific molecules further comprise a silent Fc fragment. In some embodiments, the multi-specific molecules of the present application are humanized multi-specific antibody molecules.

In some embodiments, the first targeting domain has a binding affinity to human CD3 that equals to, or is greater than, 1, 5, 10, 15, 20, 30, 40, 50, 100 or 200 nmol/L. In some embodiments, the first targeting domain has a binding affinity to human CD3 that equals to, or is greater than, 1 nmol/L. In some embodiments, the first targeting domain has a binding affinity to human CD3 that equals to, or is greater than, 15 nmol/L. In some embodiments, the first targeting domain has a binding affinity to human CD3 in the range of, 1-10, 1-50, 1-100, 1-200, 1-500, 1-1000, 1-2000, 10-50, 10-100, 10-200, 10-500, 10-1000, 10-2000, 50-100, 50-200, 50-500, 50-1000, 50-2000, 100-200, 100-500, 100-1000, 1-2000, 200-500, 200-1000, 200-2000, 500-1000, 500-2000, or 1000-2000 nmol/L. In some embodiments, the binding affinity is determined by the method described in Example 1.

In some embodiments, the first targeting domain comprises (1) a heavy chain CDR1 comprising SEQ ID NO:93, (2) a heavy chain CDR2 comprising a sequence selected from the group consisting of SEQ ID NOS: 94, 101 and 103, (3) a heavy chain CDR3 comprising a sequence selected from the group consisting of SEQ ID NOS: 95 and 105, (4) a light chain CDR1 comprising the sequence of SEQ ID NO: 96, (5) a light chain CDR2 comprising the sequence of SEQ ID NO:97, and (6) a light chain CDR3 comprising SEQ ID NO:98. In some embodiments, the targeting domain that specifically binds to human CD3 with a medium or low binding affinity comprise (1) a heavy chain variable region sequence selected from the group consisting of SEQ ID NOS: 99, 102, 104 and 106; and/or (2) a light chain variable region sequence of SEQ ID NO: 100.

In some embodiments, the second targeting domain has a binding affinity to human CD28 that equals to, or is greater than, 10, 30, 100, 200, 300, 400, 500, 600, 1000 or 2000 nmol/L. In some embodiments, the second targeting domain has a binding affinity to human CD28 that equals to, or is greater than, 200 nmol/L. In some embodiments, the first targeting domain has a binding affinity to human CD28 that equals to, or is greater than, 600 nmol/L. In some embodiments, the second targeting domain has a binding affinity to human CD28 in the range of, 10-50, 10-200, 10-500, 10-1000, 10-2000, 10-5000, 10-10000, 50-200, 50-500, 50-1000, 50-2000, 50-5000, 50-10000, 200-500, 200-1000, 200-2000, 200-5000, 200-10000, 500-1000, 500-2000, 500-5000, 500-10000, 1000-2000, 1000-5000, 1000-10000, 2000-5000, 2000-10000, or 5000-10000 nmol/L. In some embodiments, the binding affinity is determined by the method described in Example 1.

In some embodiments, the second targeting domain comprises (1) a heavy chain CDR1 comprising a sequence selected from the group consisting of SEQ ID NOS: 107 and 117 (2) a heavy chain CDR2 comprising a sequence selected from the group consisting of SEQ ID NOS: 108 and 120, (3) a heavy chain CDR3 comprising a sequence of SEQ ID NOS: 115, 118, 124, and 126, (4) a light chain CDR1 comprising the sequence of SEQ ID NO: 110, (5) a light chain CDR2 comprising the sequence of SEQ ID NO: 111, and (6) a light chain CDR3 comprising SEQ ID NO:112. In some embodiments, the targeting domain that specifically binds to human CD28 with a medium or low binding affinity comprise (1) a heavy chain variable region sequence selected from the group consisting of 116, 119, 121, 122, 123, 125 and 127; and/or (2) a light chain variable region comprising SEQ ID NO: 114.

In some embodiments, the third targeting domain is a Muc17-targeting domain. In some embodiments, the third targeting domain comprises (1) a heavy chain CDR1 comprising a sequence selected from the group consisting of SEQ ID NOS: 1, 9, 19 and 27, (2) a heavy chain CDR2 comprising a sequence selected from the group consisting of SEQ ID NOS: 2, 10, 16, 20 and 28, (3) a heavy chain CDR3 comprising a sequence selected from the group consisting of SEQ ID NOS: 3, 11, 21 and 29, (4) a light chain CDR1 comprising a sequence selected from the group consisting of SEQ ID NOS: 4, 12, 22 and 30, (5) a light chain CDR2 comprising a sequence selected from the group consisting of SEQ ID NOS: 5, 23 and 31, and (6) a light chain CDR3 comprising a sequence selected from the group consisting of SEQ ID NOS: 6, 13, 24 and 32. In some embodiments, the targeting domain that specifically binds to human Muc17 comprises (1) a heavy chain variable region sequence selected from the group consisting of 7, 14, 17, 25 and 33; and/or (2) a light chain variable region sequence selected from the group consisting of 8, 15, 18, 26 and 34.

In some embodiments, the third targeting domain is a DLL3-targeting domain. In some embodiments, the third targeting domain comprises (1) a heavy chain CDR1 comprising a sequence selected from the group consisting of SEQ ID NOS: 35, 43, 51 and 59, (2) a heavy chain CDR2 comprising a sequence selected from the group consisting of SEQ ID NOS: 36, 44, 52 and 60, (3) a heavy chain CDR3 comprising a sequence selected from the group consisting of SEQ ID NOS: 37, 45 and 53, (4) a light chain CDR1 comprising a sequence selected from the group consisting of SEQ ID NOS: 38, 46 and 54, (5) a light chain CDR2 comprising a sequence selected from the group consisting of SEQ ID NOS: 39, 47, 55 and 61, and (6) a light chain CDR3 comprising a sequence selected from the group consisting of SEQ ID NOS: 40, 48, 56 and 62. In some embodiments, the targeting domain that specifically binds to human DLL3 comprises (1) a heavy chain variable region sequence selected from the group consisting of 41, 49, 57 and 63; and/or (2) a light chain variable region sequence selected from the group consisting of 42, 50, 58 and 64.

In some embodiments, the third targeting domain is a CLDN18.2-targeting domain. In some embodiments, the third targeting domain comprises (1) a heavy chain CDR1 comprising a sequence selected from the group consisting of SEQ ID NOS: 65, 73, 80 and 86, (2) a heavy chain CDR2 comprising a sequence selected from the group consisting of SEQ ID NOS: 66, 74, 81 and 87, (3) a heavy chain CDR3 comprising a sequence selected from the group consisting of SEQ ID NOS: 67, 75, 82 and 88, (4) a light chain CDR1 comprising a sequence selected from the group consisting of SEQ ID NOS: 68, 76 and 89, (5) a light chain CDR2 comprising the sequence of SEQ ID NO:69, and (6) a light chain CDR3 comprising a sequence selected from the group consisting of SEQ ID NOS: 70, 77, 83 and 90. In some embodiments, the targeting domain that specifically binds to human CLDN18.2 comprises (1) a heavy chain variable region sequence selected from the group consisting of 71, 78, 84 and 91; and/or (2) a light chain variable region sequence selected from the group consisting of 72, 79, 85 and 92.

### 328M Molecules

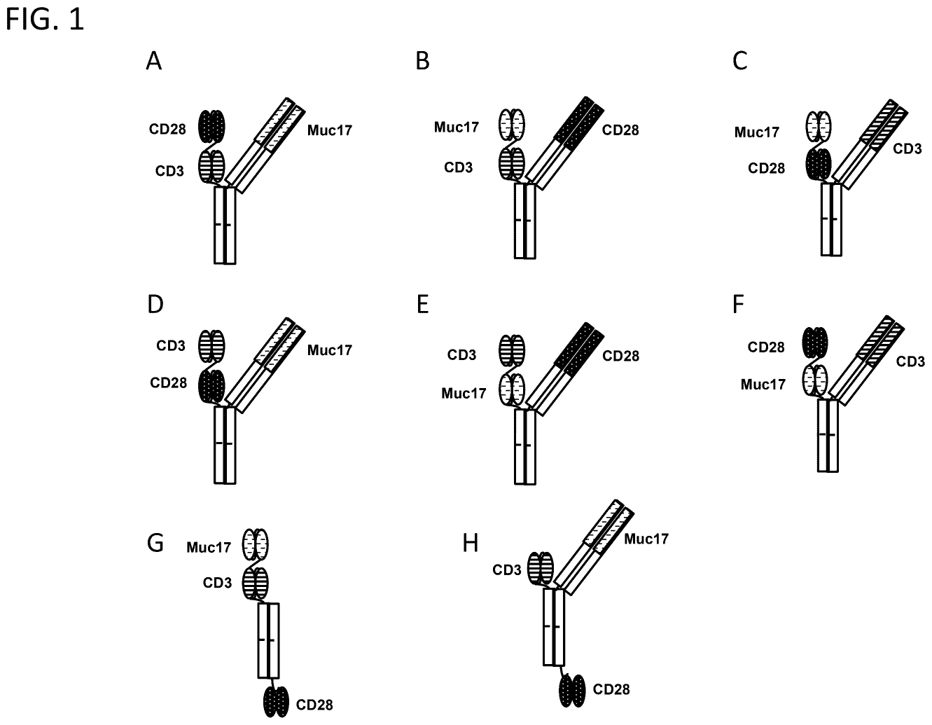

In some embodiments, the multi-specific molecule of the present application comprises a) a first targeting domain that specifically binds to human CD3 with a medium or low binding affinity; b) a second targeting domain that specifically binds to human CD28 with medium or low binding affinity; and c) a third targeting domains that specifically binds to Muc17. In some embodiments, the multi-specific molecule further comprises a silent Fc fragment. In some embodiments, the multi-specific molecule has a conformation of Muc17-scFvxCD3-scFv/CD28-Fab, as shown in FIG. 1, Panels A-H (also referred to as 328M TriTEs). The 328M molecule comprises three polypeptide chains: Chain 1 comprises, from N-terminus to C-terminus, Muc17-targeting domain in scFv form, CD3-targeting domain in scFv form, and a silent Fc sequence; Chain 2 comprises, from N-terminus to C-terminus, the heavy chain variable region sequence of a CD28-targeting Fab fragment and a silent Fc sequence; Chain 3 comprise the light chain variable region of the CD28-targeting Fab fragment. Exemplary sequences of 328M TriTEs are listed in Table 2.

TABLE 2
Exemplary 328M molecules

| Muc17 | CD3 | CD28 | | Molecule | Seq ID | Seq ID | Seq ID | CD3/CD28 |
|---|---|---|---|---|---|---|---|---|
| Antibody | Antibody | Antibody | Configuration | Name | chain 1 | chain 2 | chain 3 | affinity |
| hu1MU32A | aCD3 | aCD28 | Muc17-scFv × | 328M32 | 150 | 132 | 130 | 3/829 |
| | (VH_A116E) | (VH_C55S, | CD3-scFv/CD28- | | | | | |
| | | W113F) | Fab | | | | | |
| hu1MU32A | aCD3 | aCD28 | Muc17-scFv × | 328M55 | 128 | 129 | 130 | 3/759 |
| | (VH_A116E) | (VH_C55S, | CD3-scFv/CD28- | | | | | |
| | | L111Y, | Fab | | | | | |
| | | W113F) | | | | | | |
| hu1MU32A | aCD3 | aCD28 | Muc17-scFv × | 328M56 | 131 | 129 | 130 | 20/759 |
| | (VH_A116E, | (VH_C55S, | CD3-scFv/CD28- | | | | | |
| | Y60A) | L111Y, | Fab | | | | | |
| | | W113F) | | | | | | |
| Hu1MU32A | aCD3 | aCD28 | Muc17-scFv × | 328M57 | 131 | 132 | 130 | 20/846 |
| | (VH_A116E, | (VH_C55S, | CD3-scFv/CD28- | | | | | |
| | Y60A) | W113A) | Fab | | | | | |
| hu1MU32A | aCD3 | aCD28 | Muc17-scFv × | 328M58 | 133 | 129 | 130 | 400/759 |
| | (VH_A116D) | (VH_C55S, | CD3-scFv/CD28- | | | | | |
| | | L111Y, | Fab | | | | | |
| | | W113F) | | | | | | |
| hu1MU32A | aCD3 | aCD28 | Muc17-scFv × | 328M59 | 133 | 132 | 130 | 400/846 |
| | (VH_A116D) | (VH_C55S, | CD3-scFv/CD28- | | | | | |
| | | W113A) | Fab | | | | | |
| huMUs | aCD3 | aCD28 | Muc17-scFv × | 328M56s | 151 | 129 | 130 | 37/750 |
| | (VH_A116E, | (VH_C55S, | CD3-scFv/CD28- | | | | | |
| | Y60A) | L111Y, | Fab | | | | | |
| | | W113F) | | | | | | |
| huMUs | aCD3 | aCD28 | Muc17-scFv × | 328M58s | 152 | 129 | 130 | 400/850 |
| | (VH_A116D) | (VH_C55S, | CD3-scFv/CD28- | | | | | |
| | | L111Y, | Fab | | | | | |
| | | W113F) | | | | | | |
| hu1MU32A | aCD3 | aCD28 | Muc17-scFv × | 328M62 | 131 | 153 | 130 | 20/300 |
| | (VH_A116E, | (VH_C55S, | CD3-scFv/CD28- | | | | | |
| | Y60A) | N62S, Y109F, | Fab | | | | | |
| | | L111Y) | | | | | | |

### 328D Molecules

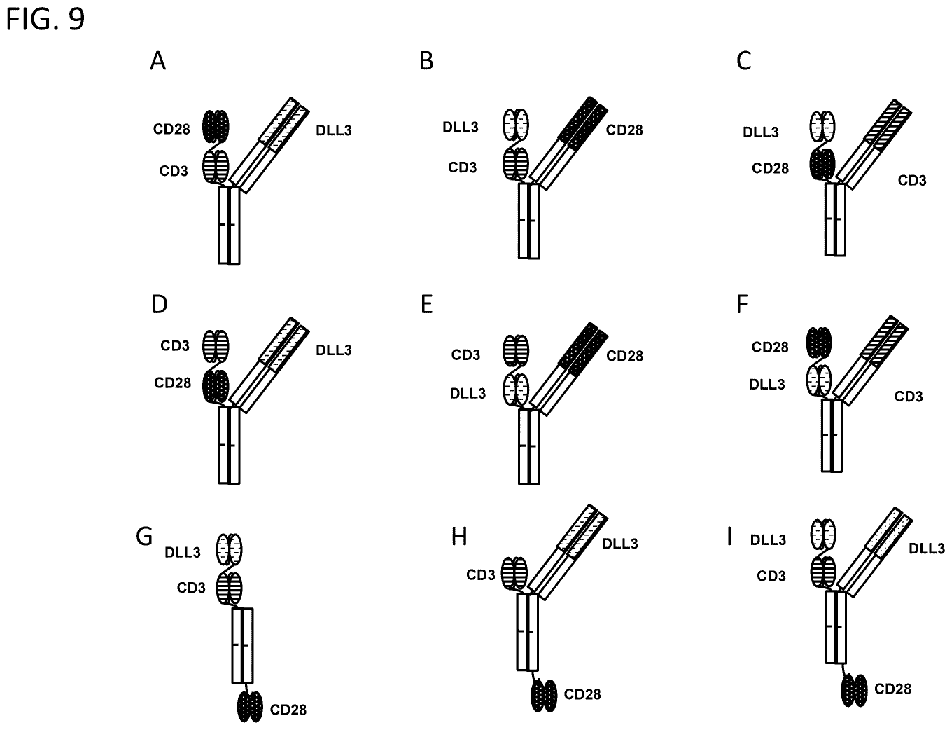

In some embodiments, the multi-specific molecule of the present application comprises (a) a) a first targeting domain that specifically binds to human CD3 with a medium or low binding affinity; b) a second targeting domain that specifically binds to human CD28 with medium or low binding affinity; and c) a third targeting domains that specifically binds to DLL3. In some embodiments, the multi-specific molecule further comprises a silent Fc fragment. In some embodiments, the multi-specific molecule has a conformation of DLL3-scFvxCD3-scFv/CD28-Fab, as shown in FIG. 9, Panels A-I (also referred to as 328D TriTEs). In some embodiments, The 328D molecules comprises three polypeptide chains: Chain 1 comprises, from N-terminus to C-terminus, DLL3-targeting domain in scFv form, CD3-targeting domain in scFv form, and a silent Fc sequence; Chain 2 comprises, from N-terminus to C-terminus, the heavy chain variable region sequence of a CD28-targeting Fab fragment and a silent Fc sequence; Chain 3 comprise the light chain variable region of the CD28-targeting Fab fragment. Exemplary sequences of 328D molecules are listed in Table 3.

TABLE 3
Exemplary 328D molecules

| DLL3 | CD3 | CD28 | | Molecule | Seq ID | Seq ID | Seq ID | CD3/CD28 |
|---|---|---|---|---|---|---|---|---|
| Antibody | Antibody | Antibody | Configuration | Name | chain 1 | chain 2 | chain 3 | affinity |
| huD139A | aCD3 | aCD28 | DLL3-scFv × | 328D18 | 134 | 135 | 130 | 20/113 |
| | (VH_A116E, | (VH_C55S, | CD3-scFv/CD28- | | | | | |
| | Y60A) | Y109F, | Fab | | | | | |
| | | L111Y) | | | | | | |
| huD139A | aCD3 | aCD28 | DLL3-scFv × | 328D19 | 134 | 129 | 130 | 20/759 |
| | (VH_A116E, | (VH_C55S, | CD3-scFv/CD28- | | | | | |
| | Y60A) | L111Y, | Fab | | | | | |
| | | W113F) | | | | | | |
| huD139A | aCD3 | aCD28 | DLL3-scFv × | 328D20 | 134 | 132 | 130 | 20/850 |
| | (VH_A116E, | (VH_C55S, | CD3-scFv/CD28- | | | | | |
| | Y60A) | W113A) | Fab | | | | | |
| huD139A | aCD3 | aCD28 | DLL3-scFv × | 328D41 | 136 | 135 | 130 | 3/113 |
| | (VH_A116E) | (VH_C55S, | CD3-scFv/CD28- | | | | | |
| | | Y109F | Fab | | | | | |
| | | L111Y) | | | | | | |
| huD139A | aCD3 | aCD28 | DLL3-scFv × | 328D42 | 136 | 129 | 130 | 3/759 |
| | (VH_A116E) | (VH_C55S, | CD3-scFv/CD28- | | | | | |
| | | L111Y, | Fab | | | | | |
| | | W113F) | | | | | | |
| huD139A | aCD3 | aCD28 | DLL3-scFv × | 328D47 | 134 | 153 | 130 | 30/300 |
| | (VH_A116E, | (VH_C55S, | CD3-scFv/CD28- | | | | | |
| | Y60A) | N62S, | Fab | | | | | |
| | | Y109F, | | | | | | |
| | | L111Y) | | | | | | |
| hD139A, | aCD3 | aCD28 | DLL3-scFv × | 328D48 | 134 | 154 | 155 | 30/100 |
| h1D4 | (VH_A116E, | (VH_C55S, | CD3-scFv/ | | | | | |
| | Y60A) | Y109F, | DLL3Fab × | | | | | |
| | | L111Y) | CD28scFv | | | | | |
| hD139A, | aCD3 | aCD28 | DLL3-scFv × | 328D49 | 134 | 156 | 155 | 30/300 |
| h1D4 | (VH_A116E, | (VH_C55S, | CD3-scFv/ | | | | | |
| | Y60A) | N62S, | DLL3Fab × | | | | | |
| | | Y109F, | CD28scFv | | | | | |
| | | L111Y) | | | | | | |
| hD139A, | aCD3 | aCD28 | DLL3-scFv × | 328D50 | 134 | 157 | 155 | 30/750 |
| h1D4 | (VH_A116E, | (VH_C55S, | CD3-scFv/ | | | | | |
| | Y60A) | L111Y, | DLL3Fab × | | | | | |
| | | W113F) | CD28scFv | | | | | |
| hD139A, | aCD3 | aCD28 | DLL3-scFv × | 328D51 | 136 | 154 | 155 | 4/100 |
| h1D4 | (VH_A116E) | (VH_C55S, | CD3-scFv/ | | | | | |
| | | Y109F, | DLL3Fab × | | | | | |
| | | L111Y) | CD28scFv | | | | | |
| hD139A, | aCD3 | aCD28 | DLL3-scFv × | 328D52 | 136 | 156 | 155 | 4/300 |
| h1D4 | (VH_A116E) | (VH_C55S, | CD3-scFv/ | | | | | |
| | | N62S, | DLL3Fab × | | | | | |
| | | Y109F, | CD28scFv | | | | | |
| | | L111Y) | | | | | | |
| hD139A, | aCD3 | aCD28 | DLL3-scFv × | 328D53 | 136 | 157 | 155 | 4/750 |
| h1D4 | (VH_A116E) | (VH_C55S, | CD3-scFv/ | | | | | |
| | | L111Y, | DLL3Fab × | | | | | |
| | | W113F) | CD28scFv | | | | | |
| hD139A, | aCD3 | aCD28 | DLL3-scFv × | 328D54 | 136 | 158 | 155 | 4/1000 |
| h1D4 | (VH_A116E) | (VH_C55S, | CD3-scFv/ | | | | | |
| | | W113A) | DLL3Fab × | | | | | |
| | | | CD28scFv | | | | | |
| hD139A | aCD3 | aCD28 | DLL3-scFv × | 328D55 | 136 | 153 | 130 | 4/300 |
| | (VH_A116E) | (VH_C55S, | CD3-scFv/ | | | | | |
| | | N62S, | CD28-Fab | | | | | |
| | | Y109F, | | | | | | |
| | | L111Y) | | | | | | |

### 328C Molecules

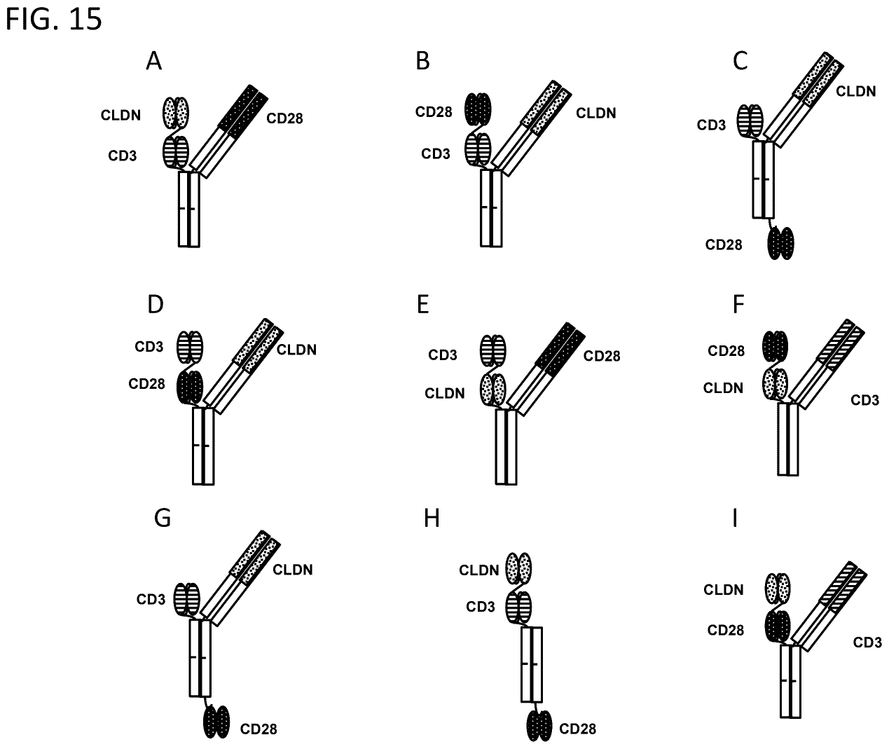

In some embodiments, the multi-specific molecule of the present application comprises (a) a) a first targeting domain that specifically binds to human CD3 with a medium or low binding affinity; b) a second targeting domain that specifically binds to human CD28 with medium or low binding affinity; and c) a third targeting domains that specifically binds to CLDN. In some embodiments, the multi-specific molecule further comprises a silent Fc fragment.). In some embodiments, the multi-specific molecules are humanized. In some embodiments, the multi-specific molecule has a conformation of CLDN-scFvxCD3-scFv/CD28-Fab, as shown in FIG. 15, Panels A-I (also referred to as 328C TriTEs). In some embodiments, The 328C molecules comprises three polypeptide chains: Chain 1 comprises, from N-terminus to C-terminus, CLDN-targeting domain in scFv form, CD3-targeting domain in scFv form, and a silent Fc sequence; Chain 2 comprises, from N-terminus to C-terminus, the heavy chain variable region sequence of a CD28-targeting Fab fragment and a silent Fc sequence; Chain 3 comprise the light chain variable region of the CD28-targeting Fab fragment. Exemplary sequences of 328C molecules are listed in Table 4.

TABLE 4
Exemplary 328C molecules

| CLDN18.2 | CD3 | CD28 | | Molecule | Seq ID | Seq ID | Seq ID |
|---|---|---|---|---|---|---|---|
| Antibody | Antibody | Antibody | Configuration | Name | chain 1 | chain 2 | chain 3 |
| huCldn29B | aCD3 | aCD28 | CLDN18.2- | 328C8 | 137 | 129 | 130 |
| | (VH_A116E) | (VH_C55S, | scFv × | | | | |
| | | L111Y, | CD3-scFv/ | | | | |
| | | W113F) | CD28-Fab | | | | |
| huCldn29B | aCD3 | aCD28 | CLDN18.2- | 328C9 | 139 | 129 | 130 |
| | (VH_A116E) | (VH_C55S, | scFv × | | | | |
| | Y60A) | L111Y, | CD3-scFv/ | | | | |
| | | W113F) | CD28-Fab | | | | |
| huCldn29B | aCD3 | aCD28 | CD28-scFv × | 328C12 | 140 | 141 | 138 |
| | (VH_A116E) | (VH_C55S, | CD3-scFv/ | | | | |
| | | L111Y, | CLDN18.2 Fab | | | | |
| | | W113F) | | | | | |
| huCldn29B | aCD3 | aCD28 | CD28-scFv × | 328C13 | 142 | 141 | 138 |
| | (VH_A116E, | (VH_C55S, | CD3-scFv/ | | | | |
| | Y60A) | L111Y, | CLDN18.2 Fab | | | | |
| | | W113F) | | | | | |
| huCldn29B | aCD3 | aCD28 | CD3-scFv/ | 328C14 | 143 | 144 | 138 |
| | (VH_A116E) | (VH_C55S, | CLDN18.2- | | | | |
| | | L111Y, | Fab × | | | | |
| | | W113F) | CD28-scFv | | | | |
| huCldn29B | aCD3 | aCD28 | CD3-scFv/ | 328C15/17 | 145 | 144 | 138 |
| | (VH_A116E, | (VH_C55S, | CLDN18.2- | | | | |
| | Y60A) | L111Y, | Fab × | | | | |
| | | W113F) | CD28-scFv | | | | |
| huCldn29B | aCD3 | aCD28 | CD3-scFv/ | 328C18 | 159 | 160 | 138 |
| | (VH_A116E, | (VH_C55S, | CLDN18.2- | | | | |
| | Y60A) | N62S, | Fab × | | | | |
| | | Y109F, | CD28-scFv | | | | |
| | | L111Y) | | | | | |
| huCldn29B | aCD3 | aCD28 | CD3-scFv/ | 328C19 | 145 | 160 | 138 |
| | (VH_A116E, | (VH_C55S, | CLDN18.2- | | | | |
| | K59E, | N62S, | Fab × | | | | |
| | Y60A) | Y109F, | CD28-scFv | | | | |
| | | L111Y) | | | | | |
| hCldn29B | aCD3 | aCD28 | CD28-scFv × | 328C20 | 161 | 141 | 138 |
| | (VH_A116E, | (VH_C55S, | CD3-scFv/ | | | | |
| | Y60A) | N62S, | CLDN18.2 | | | | |
| | | Y109F, | Fab | | | | |
| | | L111Y) | | | | | |

### III. Nucleic Acids and Host Cells for Expressing Multi-Specific Molecules of the Present Application

Another aspect of the present application relates to nucleic acids encoding one or more polypeptide chains of the multi-specific molecule of the present application, as well as expression vectors comprising such nucleic acids. In particular, the nucleic acids encode one or more HCDRs, LCDRs, HCVRs and/or LCVRs corresponding to any of the antibodies, multi-specific molecules or fragments described herein.

In another aspect, the present application provides one or more expression vectors comprising the one or more nucleic acids encoding one or more polypeptide chains of the multi-specific molecule of the present application.

DNA(s) encoding antigen binding sites can be isolated and sequenced from a monoclonal antibody produced in hybridoma cells using conventional procedures (e.g., by using oligonucleotide probes that are capable of binding specifically to genes encoding the heavy and light chains of the monoclonal antibodies). Alternatively, amino acid sequences from immunoglobulins of interest may be determined by direct protein sequencing, and suitable encoding nucleotide sequences can be designed according to a universal codon table. In other cases, nucleotide and amino acid sequences of antigen binding sites or other immunoglobulin sequences, including constant regions, hinge regions and the like may be obtained from published sources well known in the art.

Expression vectors encoding a particular multi-specific molecule or a polypeptide chain of a particular multi-specific molecule may be used to produce the particular multi-specific molecule in cultured cells in vitro, or they may be directly administered to a patient to express the particular multi-specific molecule in vivo or ex vivo. As used herein, an “expression vector” refers to a viral or non-viral vector comprising a polynucleotide encoding one or more polypeptide chains corresponding to a particular multi-specific molecule in a form suitable for expression from the polynucleotide(s) in a host cell for preparation of the multi-specific molecule or for direct administration as a therapeutic agent.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

Nucleic acid sequences for expressing the particular multi-specific molecule typically include an amino terminal signal peptide sequence, which is removed from the mature protein. Since the signal peptide sequences can affect the levels of expression, the polynucleotides may encode any one of a variety of different N-terminal signal peptide sequences. It will be appreciated by those skilled in the art that the design of the expression vector can depend on such factors as the choice of the host cell to be transformed, the level of expression of protein desired, and the like.

The above described “regulatory sequences” refer to DNA sequences necessary for the expression of an operably linked coding sequence in one or more host organisms. The term “regulatory sequences” is intended to include promoters, enhancers and other expression control elements (e.g., polyadenylation signals). Regulatory sequences include those which direct constitutive expression of a nucleotide sequence in many types of host cells or those which direct expression of the nucleotide sequence only in certain host cells (e.g., tissue-specific regulatory sequences). Expression vectors generally contain sequences for transcriptional termination, and may additionally contain one or more elements positively affecting mRNA stability.

The expression vector contains one or more transcriptional regulatory elements, including promoters and/or enhancers, for directing the expression of antitumor antagonists. A promoter comprises a DNA sequence that functions to initiate transcription from a relatively fixed location in regard to the transcription start site. A promoter contains core elements required for basic interaction of RNA polymerase and transcription factors, and may operate in conjunction with other upstream elements and response elements.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together. An expression vector may be designed to facilitate expression of the particular multi-specific molecule in one or more cell types.

To co-express the individual chains of a multi-specific molecule, a suitable splice donor and splice acceptor sequences may be incorporated for expressing both products. Alternatively, an internal ribosome binding sequence (IRES) or a 2A peptide sequence, may be employed for expressing multiple products from one promoter. An IRES provides a structure to which the ribosome can bind that does not need to be at the 5′ end of the mRNA. It can therefore direct a ribosome to initiate translation at a second initiation codon within a mRNA, allowing more than one polypeptide to be produced from a single mRNA. A 2A peptide contains short sequences mediating co-translational self-cleavage of the peptides upstream and downstream from the 2A site, allowing production of two different proteins from a single transcript in equimolar amounts. CHYSEL is a non-limiting example of a 2A peptide, which causes a translating eukaryotic ribosome to release the growing polypeptide chain that it is synthesizing without dissociating from the mRNA. The ribosome continues translating, thereby producing a second polypeptide.

An expression vector may comprise a viral vector or a non-viral vector. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like. A non-viral vector is simply a “naked” expression vector that is not packaged with virally derived components (e.g., capsids and/or envelopes). Examples of non-viral vectors include, but are not limited to, plasmid vectors.

### IV. Pharmaceutical Compositions

Another aspect of the present application relates to a pharmaceutical composition comprising (1) a multi-specific molecule of the present application, or (2) one or more expression vectors encoding a multi-specific molecule of the present application. In some embodiments, the pharmaceutical composition comprises a multi-specific molecule of the present application and a pharmaceutically acceptable carrier. In some embodiments, the pharmaceutical composition comprises one or more expression vectors encoding a multi-specific molecule of the present application and a pharmaceutically acceptable carrier.

The pharmaceutical composition of the present application can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

The pharmaceutical composition of the present application can be lyophilized and stored as sterile powders, preferably under vacuum, and then reconstituted in bacteriostatic water (containing, for example, benzyl alcohol preservative) or in sterile water prior to injection.

### V. Methods for Activating T Cells and Methods of Treatment

Another aspect of the present application relates methods for activating T cells with the pharmaceutical composition of the present application.

Another aspect of the present application relates methods for treating cell proliferative disorder in a subject. In some embodiments, the method comprises the step of administering to a subject in need thereof an effective amount of a pharmaceutical composition of the present application. In some embodiments, the cell proliferative disorder is a cancer or tumor. Examples of cancer or tumor include, but are not limited to, squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (H L), non-Hodgkin's lymphoma (NEIL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

Any suitable route or mode of administration can be employed for providing the patient with a therapeutically or prophylactically effective dose of the pharmaceutical composition of the present application. Exemplary routes or modes of administration include parenteral {e.g., intravenous, intraarterial, intramuscular, subcutaneous, intratumoral), oral, topical (nasal, transdermal, intradermal or intraocular), mucosal {e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

The active ingredient (e.g., a multi-specific molecule of the present application, or an expression vectors encoding a multi-specific molecule of the present application) in the pharmaceutical compositions may be formulated in an “effective amount”. An “effective amount” refers to an amount effective, at dosages and for periods of time necessary, to achieve the desired therapeutic result. An effective amount of the active ingredient may vary depending on the condition to be treated, the severity and course of the condition, the mode of administration, whether the antibody or agent is administered for preventive or therapeutic purposes, the bioavailability of the particular multi-specific molecule, the ability of the multi-specific molecule to elicit a desired response in the individual, previous therapy, the age, weight and sex of the patient, the patient's clinical history and response to the multi-specific molecule, the type of the multi-specific molecule used, discretion of the attending physician, etc. An effective amount is also one in which any toxic or detrimental effects of the multi-specific molecule or expression vector(s) encoding the multi-specific molecule is outweighed by the therapeutically beneficial effects.

Preferably, the polypeptide domains in the multi-specific molecule are derived from the same host in which they are to be administered in order to reduce inflammatory responses against the administered multi-specific molecule.

The pharmaceutical composition of the present application is suitably administered to a patient at one time or over a series of treatments and may be administered to the patient at any time from diagnosis onwards. The pharmaceutical composition of the present application may be administered as the sole treatment or in conjunction with other drugs or therapies useful in treating the condition in question.

As a general proposition, an effective amount of the multi-specific molecule of the present application is administered in a range from about 1 ng/kg body weight/day to about 100 mg/kg body weight/day whether by one or more administrations. In a particular embodiment, each multi-specific molecule of the present application is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, each multi-specific molecule is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The multi-specific molecule of the present application may be administered daily, every 2, 3, 4, 5, 6 or 7 days, or every 1, 2, 3 or 4 weeks.

Dosages can be tested in several art-accepted animal models suitable for any particular cell proliferative disorder.

Delivery methodologies may also include the use of polycationic condensed DNA linked or unlinked to killed viruses, ligand linked DNA, liposomes, eukaryotic cell delivery vehicles cells, deposition of photopolymerized hydrogel materials, use of a handheld gene transfer particle gun, ionizing radiation, nucleic charge neutralization or fusion with cell membranes, particle mediated gene transfer and the like.

### VI. Methods for Producing Multi-Specific Molecules of the Present Application

Another aspect of the present application relates to a method for producing a multi-specific molecule of the present application. The multi-specific molecules of the present application can be generated by chemical cross-linking or by the hybrid hybridoma technology. Alternatively, multi-specific molecules of the present application can be produced by recombinant techniques, for example by linking 2 scFv molecules together with a short linker. For example, VH1-Linker1-VL1-Linker2-VH2-Linker3-VL2. With Linker1 and Linker3 having lengths between 15-30 amino acids and Linker2 being 5-10 amino acids in length. Linkers may be composed of a variety of amino acids, for example repeating units of GGGGS (SEQ ID No: 146), GKPGS (SEQ ID No: 147), GEPGS (SEQ ID No: 148), and/or GGPGS (SEQ ID No:149). Dimerization across 2 scFv molecules can be promoted by reducing the length of the linker joining the VH and the VL domain from about 15 amino acids, routinely used to produce scFv fragments, to about 5 amino acids. These linkers favor intrachain assembly of the VH and VL domains, with the configuration VH1-linker1-VL2-Linker2-VH2-Linker 3VL1 and linkers 1 and 3 being 5 amino acids in length. Any suitable short linker can be used. Thus, two fragments assemble into a dimeric molecule. Further reduction of the linker length to 0-2 amino acids can generate trimeric (triabodies) or tetrameric (tetrabodies) molecules.

In some embodiments, the method comprises the step of generating one or more expression vectors encoding one or more polypeptide chains in a multi-specific molecule of the present application, introducing the one or more expression vectors into a host cell, culturing the host cell harboring the one or more expression vectors to express the one or more polypeptide chains of the multi-specific molecule; and purifying the multi-specific molecule from the cultured cells. Any cell capable of producing a functional multi-specific molecule can be used. In preferred embodiments, the multi-specific molecule-expressing cell is of eukaryotic or mammalian origin, preferably a human cell or Chinese hamster cell. Cells from various tissue cell types may be used to express the multi-specific molecule. In other embodiments, the cell is a yeast cell, an insect cell or a bacterial cell. Preferably, the multi-specific molecule-producing cell is stably transformed with one or more expression vectors expressing all the polypeptide chains of the multi-specific molecule.

One or more expression vectors encoding the polypeptide chains of the multi-specific molecule can be introduced into a cell by any conventional method, such as by naked DNA technique, cationic lipid-mediated transfection, polymer-mediated transfection, peptide-mediated transfection, virus-mediated infection, physical or chemical agents or treatments, electroporation, etc. In addition, cells may be transfected with one or more expression vectors for expressing the multi-specific molecule along with a selectable marker facilitating selection of stably transformed clones expressing the multi-specific molecule. The multi-specific molecule produced by such cells may be collected and/or purified according to techniques known in the art, such as by centrifugation, chromatography, Protein-A or Protein-G immunoaffinity purification, etc. In some embodiments, multi-specific molecules of the present application are engineered for secretion into culture supernatants for isolation therefrom.

Exemplary multi-specific molecule-expressing cells include human Jurkat, human embryonic kidney (HEK) 293, Chinese hamster ovary (CHO) cells, mouse WEHI fibrosarcoma cells, as well as unicellular protozoan species, such as Leishmania tarentolae. In addition, stably transformed, antibody producing cell lines may be produced using primary cells immortalized with c-myc or other immortalizing agents.

In some embodiments, the multi-specific molecules of the present application are humanized to administration to human patients. Methods for humanizing antibodies or multi-specific antibodies/molecules are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

### Example 1: Methods for Determining CD3, CD28 and TAA Binding Affinity

To determine the affinity of various molecules for human CD3 or human CD28, a bio-layer interferometry (BLI) assay was performed. In this assay, a heterodimeric construct of human CD3 epsilon and CD3 delta domains (ACROBiosystems CAT #CDD-H52Wa) with a C-terminal his-tag was diluted in kinetics assay buffer to 1.5 μg/mL and loaded to anti-His assay probes. Loaded probes were then assayed for binding against serial dilutions of a test molecule (e.g., a multi-specific molecule of the present application). In a typical experiment, fresh probes were loaded with human CD3 for 150 seconds, followed by a 60 second baseline step, a 120 second association step, and a 120 second dissociation step. Each experiment included a reference probe for background subtraction, where CD3 was loaded to the reference probe but assayed for binding against buffer instead of protein. The resulting binding curves following background subtraction were aligned and processed using Savitzky-Goley filtering to generate the final binding curves, which were fit using a global, 1:1 binding model. Affinity constants were generated from the calculated kon and koff rates. The experiment was performed at 25° C. with a shaking speed of 1000 rpm.

The BLI method and the CD3 antigen used here for determining CD3 affinity may differ from the method reported by other sources for measuring their CD3 engagers. For example, the published CD3 affinity for the benchmark BiTE, AMG 757 (Tarlatamab), was measured using surface plasmon resonance (SPR), and the antigen was the CD3 epsilon N-terminal peptide (aa 1-27) fused to chicken albumin. The results reported here use BLI instead of SPR and the antigen contains the CD3 epsilon and CD3 delta domains as a heterodimer (Seung et al., Nature. 603:328-334 (2022)). These technical differences may result in KD values that are different from the published values. Thus, the affinity values associated with the claims of this patent should be measured with a protocol that is comparable to the one described here. CD28 affinity was determined in a similar manner. Human CD28-his (ACROBiosystems Cat #CD8-H52H3) was loaded to anti-His probes and assayed for binding against serial dilutions of test molecules. CD3 kinetics assay buffer: 1×PBS, 0.1% BSA, 0.02% Tween-20, 0.05% sodium azide. CD28 kinetics assay buffer: 50 mM sodium phosphate, 200 mM sodium chloride, 0.1% BSA, 0.02% Tween-20, 0.05% sodium azide, pH 7.4. Table 5 shows binding affinity of exemplary 328M molecules. The reagents used in the affinity assay are listed in Table 6.

TABLE 5
Binding affinity of exemplary 328M molecules

| | | Fold reduction in | |
|---|---|---|---|
| | CD3 KD (nM) | CD3 affinity | CD28 KD (nM) |
| | (measured with | compared to | (measured with |
| | Gensun method) | Tarlatamab | Gensun method) |
| AMG757 | 2 (15, | 1 | |
| (Tarlatamab, | by Amgen) | | |
| BM) | | | |
| 328M5 | 5 | 2.5 | 1 |
| 328M41 | 25 | 12.5 | 105 |
| 328M55 | 5 | 2.5 | 759 |
| 328M56 | 34 | 17 | 710 |
| 328M57 | 31 | 15.5 | 1020 |
| 328M58 | 400 | 200 | 748 |

TABLE 6
List of reagents

| | Construct/ | | |
|---|---|---|---|
| | Sequence | vendor | Cat# |
| Human CD3 | CD3E (23-126) | ACROBiosystems | CDD-H52Wa |
| E/D dimer | | | |
| | CD3D (22-105) | ACROBiosystems | CDD-H52Wa |
| Human CD28 | CD28 (19-152) | | CD8-H52H3 |
| Anti-His probes | | GatorBio | 160009 |

To evaluate the ability of anti-Muc17/DLL3/CLDN18.2 antibodies to bind to a target cell, serial dilutions of the antibodies were added to CHO-K1 cells (20,000 cells/well) over-expressing human or cyno Muc17/DLL3/CLDN18.2. The mixtures were incubated at 4° C. for 20 minutes, washed 3 times, and stained with the secondary antibody, PE labeled F(ab′) 2-Goat anti-human IgG Fc (Thermo H10104) at 4° C. for 20 minutes. Cells were washed and resuspended in 7-Amino-Actinomycin D (7-AAD) solution and fixed in 10% neutral buffered formalin solution for 15 minutes before analysis with the iQue Intellicyt system.

### Example 2: Generation of Anti-CD3 Antibodies with Medium or Low Binding Affinity

The parental anti-CD3 antibody, described in the previous patent (“Bispecific T Cell Engager” U.S. Pat. No. 11,976,133B2), provided the basis for further modifications. Genes encoding the VH and VL domains were synthesized and assembled into a single-chain variable fragment (scFv) format. To mitigate adverse reactions, such as cytokine release syndrome (CRS) caused by TAA-independent binding and T cell activation, specific mutations were introduced into the VH/VL domain of the anti-CD3 antibody to reduce its binding affinity to CD3. These mutations were designed and implemented using the QuikChange Lightning Multi Site-Directed Mutagenesis Kit (Agilent 210519) following the manufacturer's protocols. The mutated plasmid DNA sequences were verified through standard sequencing methods. Subsequently, the mutated CD3 constructs were expressed, purified, and their binding affinities were assessed. Constructs with the desired CD3 binding affinities were selected for further development. Examples of engineered CD3 variants and affinities are summarized in Table 7.

TABLE 7
Example of engineered CD3 variants and affinities

| | Mutations | Koff(1/s) | Kon(1/Ms) | KD(M) | KD (nM) |
|---|---|---|---|---|---|
| D17 | WT/A116E | 4.53E−03 | 1.03E+06 | 4.40E−09 | 4.4 |
| D18 | A116E, Y60A | 1.75E−02 | 6.40E+05 | 2.74E−08 | 27.4 |
| D43 | A116E, K59E, | 2.95E−02 | 4.15E+05 | 7.12E−08 | 71.2 |
| | Y60A | | | | |
| D44 | A116E, K59E, | 3.09E−02 | 4.02E+05 | 7.70E−08 | 77.0 |
| | Y60A | | | | |
| D45 | A116E, K59E, | 2.73E−02 | 4.40E+05 | 6.20E−08 | 62.0 |
| | Y60A | | | | |
| D46 | A116E, K59E, | 2.88E−02 | 4.34E+05 | 6.64E−08 | 66.4 |
| | Y60A | | | | |
| D19 | A116E, Y60A | 1.92E−02 | 6.13E+05 | 3.12E−08 | 31.2 |
| D20 | A116D | 7.44E−02 | 1.82E+05 | 4.10E−07 | 410 |
| M55 | WT/A116E | 4.53E−03 | 8.25E+05 | 5.49E−09 | 5.49 |
| M56 | A116E, Y60A | 2.22E−02 | 6.01E+05 | 3.69E−08 | 36.9 |
| M58 | A116D | 7.75E−02 | 1.93E+05 | 4.02E−07 | 402 |

### Example 3: Generation of Anti-CD28 Antibodies with Medium or Low Binding Affinity

The parental anti-CD28 antibody, described in the previous patent (“Trispecific T Cell Engager” U.S. Ser. No. 17/164,696), served as the foundation for further modification. Genes encoding the VH and VL domains were synthesized and formatted for expression as either a single-chain variable fragment (scFv) or a Fab fragment. To mitigate adverse reactions, such as cytokine release syndrome (CRS) resulting from TAA-independent binding and activation of T cells, mutations were introduced into the VH domain of the anti-CD28 antibody to reduce its binding affinity to CD28. These mutations were implemented using the QuikChange Lightning Multi Site-Directed Mutagenesis Kit (Agilent 210519) in accordance with the manufacturer's protocols. The mutated plasmid DNA sequences were verified through standard sequencing methods. Subsequently, the mutated anti-CD28 constructs were expressed, purified, and their binding affinities were assessed. Constructs with the desired CD28 binding affinities were selected for further development. Examples of engineered CD28 variants and affinities are summarized in Table 8.

TABLE 8
Example of engineered CD28 variants and affinities

| | | | Kon(1/ | | KD |
|---|---|---|---|---|---|
| | Mutations | Koff(1/s) | Ms) | KD(M) | (nM) |
| 328D17 | WT | 2.73E−04 | 1.87E+05 | 1.46E−09 | 1.46 |
| 328D18 | Y109F, L111Y | 2.70E−02 | 2.36E+05 | 1.14E−07 | 114 |
| 328D43 | L111A, S36G | 6.38E−02 | 2.50E+05 | 2.55E−07 | 255 |
| 328D44 | L111A, N62S | 4.51E−02 | 2.81E+05 | 1.61E−07 | 161 |
| 328D45 | Y109F, L111Y, | 8.15E−02 | 2.84E+05 | 2.87E−07 | 287 |
| | S36G | | | | |
| 328D46 | Y109F, L111Y, | 7.34E−02 | 2.65E+05 | 2.77E−07 | 277 |
| | N62S | | | | |
| 328D19 | L111Y, W113F | 3.93E−02 | 9.46E+04 | 4.15E−07 | 415 |
| 328M5 | WT | 3.81E−04 | 2.19E+05 | 1.74E−09 | 1.74 |
| 328M41 | Y109F, L111Y | 1.53E−02 | 1.46E+05 | 1.05E−07 | 105 |
| 328M56 | L111Y, W113F | 5.55E−02 | 8.77E+04 | 6.33E−07 | 633 |
| 328M57 | W113A | 2.10E−01 | 2.06E+05 | 1.02E−06 | 1020 |

### Example 4: Multi-Specific Molecules of with CD3-, CD28- and Muc17-Targeting Domains and Low-Medium CD3/CD28 Binding Affinity (Second Generation 328M TriTEs) Show Significant T-Cell Activation and Anti-Tumor Potency with Reduced Toxicity

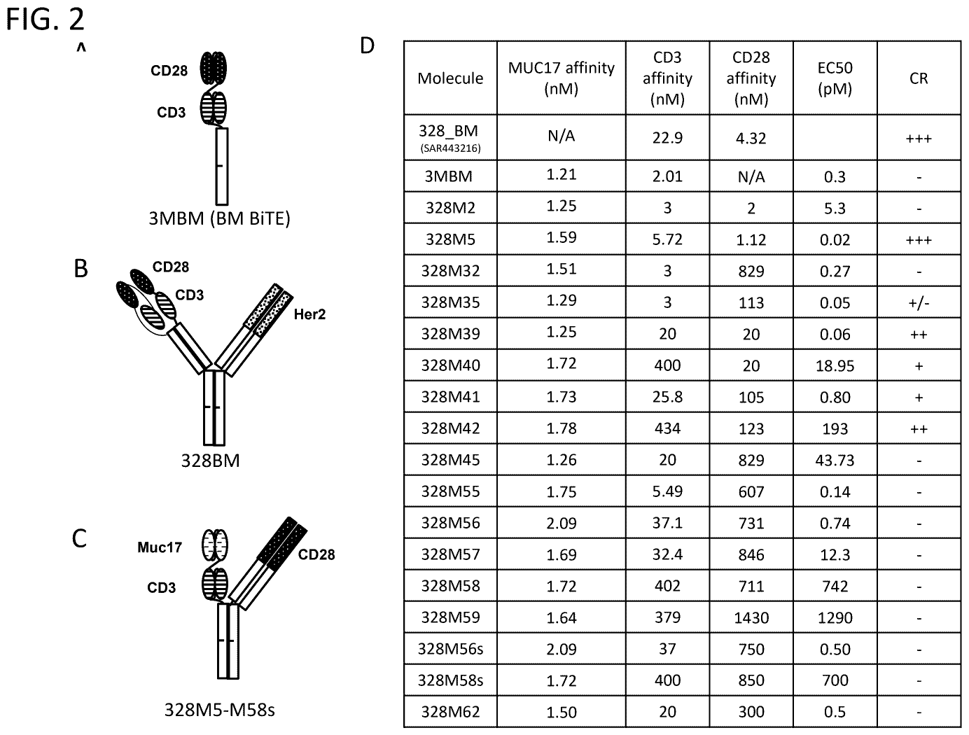

FIG. 1 depicts exemplary configurations of multi-specific molecules of the 328M second generation TriTEs, (FIG. 1, Panels A-H). FIG. 2 shows target binding affinities of embodiments of benchmark molecules containing only CD3 targeting domains, 3MBM (Panel A); benchmark molecules containing CD3-, CD28- and non-Muc17-targeting domains with high affinity to CD3/CD28, 328BM SAR443216 (Panel B); multi-specific molecules containing CD3-, CD28- and Muc17-targeting domains with high affinity to CD3/CD28, a first-generation 328M TriTEs, e.g., 328M5 and the new second generation 328M TriTEs (Panel C). Panel D shows T cell dependent cellular cytotoxicity (TDCC) potency and non-target dependent cytokine secretions.

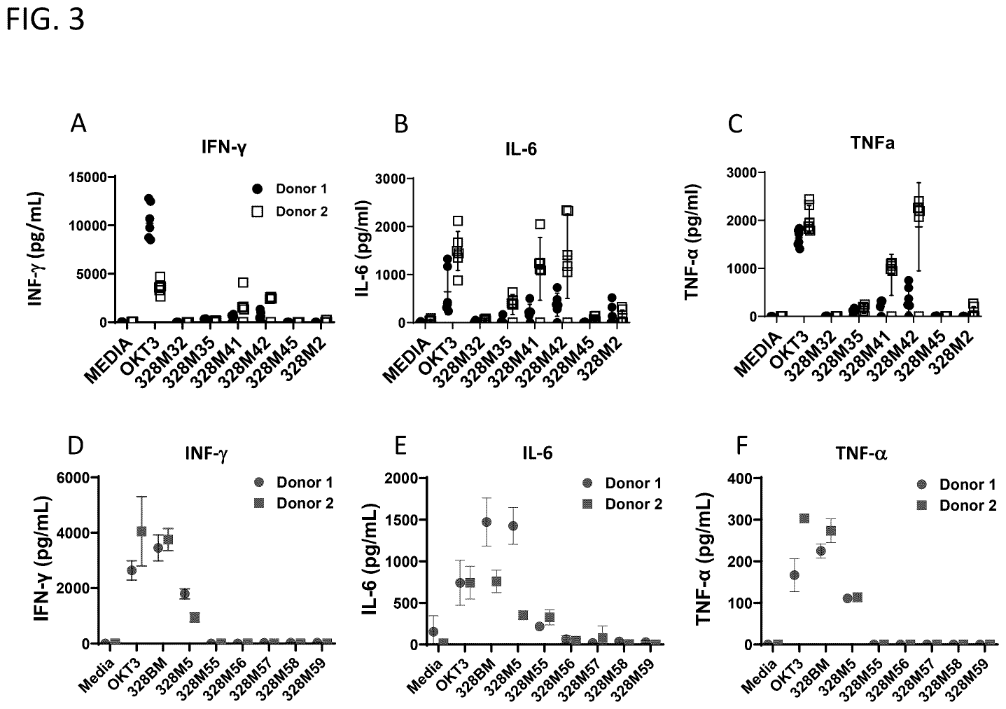

FIG. 3 Presents examples of results of target-independent cytokine release assay (CRA) compared to controls such as OKT3, the benchmark molecule 328BM and a first generation TriTE 328M5 described in previous patent (“Trispecific T Cell Engager U.S. Ser. No. 17/164,696). While some new TriTEs exhibit significant cytokine release, certain second-generation 328M TriTEs shows reduced target independent cytokine secretions compared to both benchmark molecules and the first generation 328M TriTE (FIG. 3).

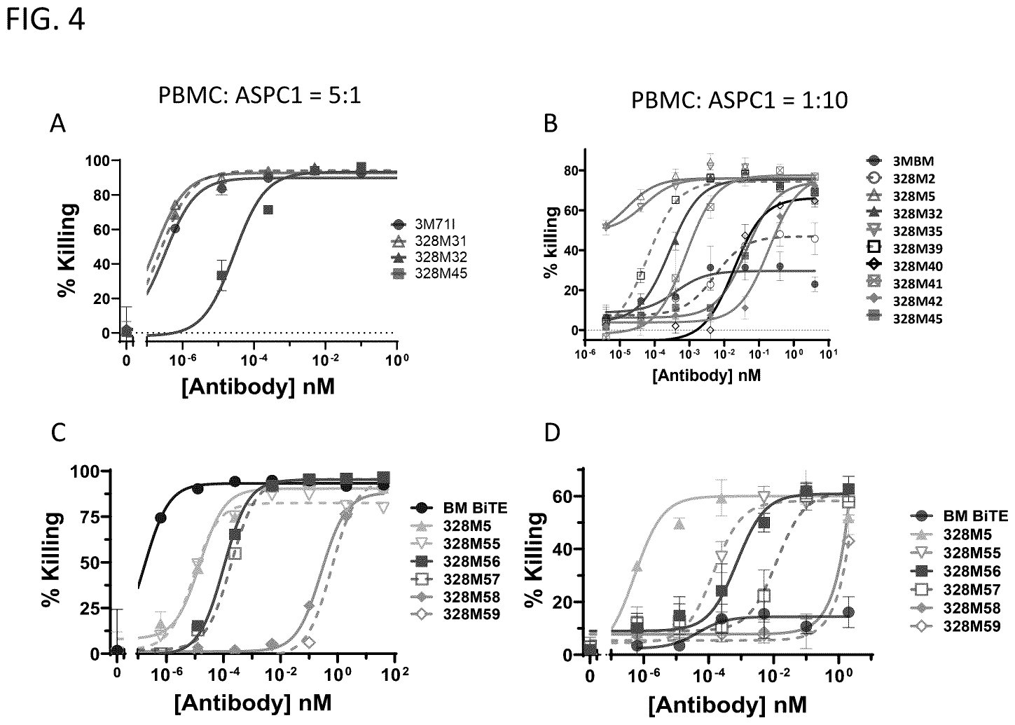

FIG. 4 shows examples of results of T Cell Dependent Cellular Cytotoxicity (TDCC) assays under varying conditions. The assays were conducted with either a high ratio of PBMC to Target ASPC1 cells (5:1) and a short incubation time of 24-48 hours (Panel A and C), or a low ratio of PBMC to Target ASPC1 cells (1:10) and an extended incubation time of 5-7 days (Panel B and D). When using a high effector-to-target ratio and with short incubation time, the activities are mainly driven by signal 1, i.e. CD3 activities. However, under low effector-to-target cell ratios and extended assay times, signal 2, i.e. CD28 activities, plays a critical roles in enhancing in TriTE-induced T cell activation and proliferation, resulting in significant killing activity. Whereas BITEs, which lack of CD28 component, exhibit minimal killing activity under these conditions.

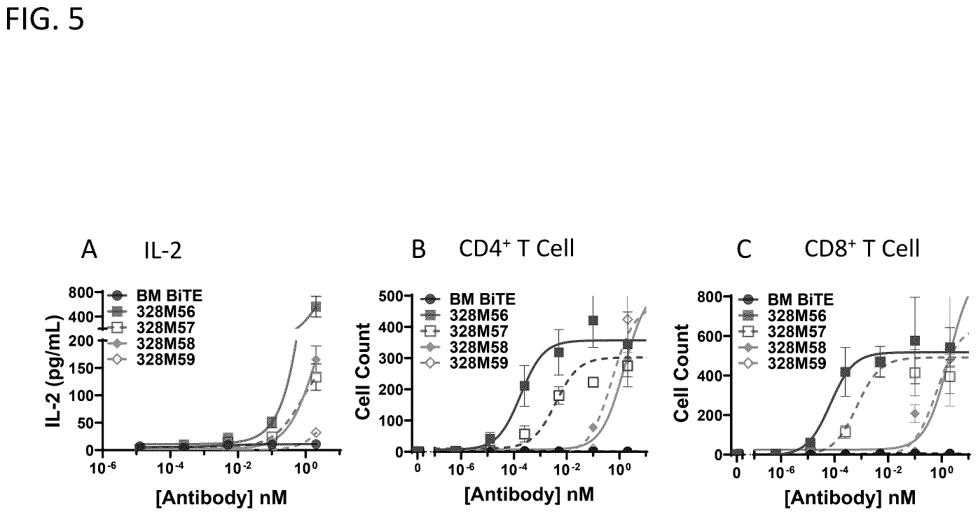

FIG. 5 shows that the second generation 328M TriTEs stimulate IL-2 secretion and T cell activation/proliferation upon target engagement when using low effector-to-target ratio and extended assay times. TriTEs induce significantly IL-2 secretion and T cell activation despite low CD3/CD28 affinity, while high-affinity BiTE shows minimal activity. The result highlights the role of signal 2, CD28 activities in T cell functions.

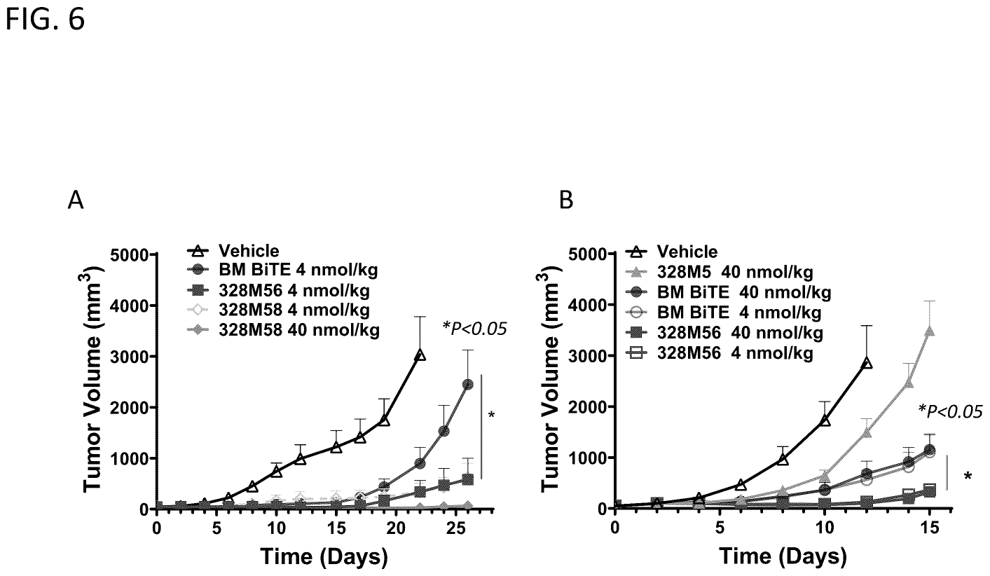

FIG. 6 shows that second-generation TriTE outperform BM BiTE and the first-generation TriTE 328M5 in a hCD3/hCD28 KI mouse model bearing B16F10-hMuc17+ tumors (Biocytogen CRO). TriTEs demonstrated superior tumor-killing activities than BM BiTE, despite their CD3 affinities being significantly lower than that of BM. Interestingly, a TriTE with substantially lower CD3 and CD28 affinities than the first generation 328M TriTE (328M5) exhibited drastically improved anti-tumor activity, contrary to their rank order by in vitro TDCC potency. This is likely due to the high affinity of CD3 and CD28 in 328M5 causing non-target dependent binding to T cells rather than target- or tumor-dependent binding. In contrast, second-generation TriTEs may target tumor cells more specifically due to their reduced affinities for CD3 and CD28. This result is unexpected as it contradicts the in vitro TDCC assay findings. Nevertheless, the second-generation TriTEs not only minimize CRS toxicity but also exhibit significant tumor-killing effects in vivo despite their significantly reduced CD3/CD28 affinities.

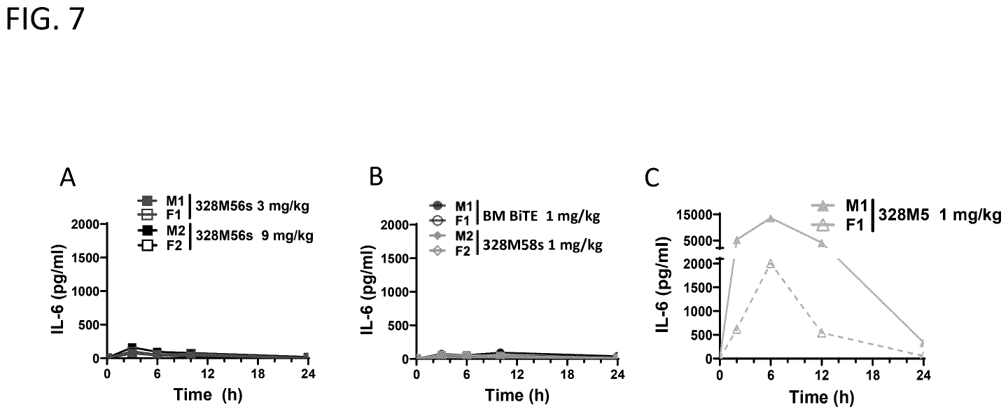

FIG. 7 shows cynomolgus monkey toxicity study of the T cell engagers. The second generation 328M TriTEs induce minimal IL-6 secretion compared to first-generation TriTE (328M5) and match clinical BM BITE. Weak IL-6 release observed for the second generation 328M TriTEs following initial administration at dose up to 9 mg/kg, with an IL-6 profile comparable to that of a clinical benchmark BiTE at 1 mg/kg (Panels A and B). In contrast, a first generation TriTE (328M5) induced high level of IL-6 at 1 mg/kg (Panel C).

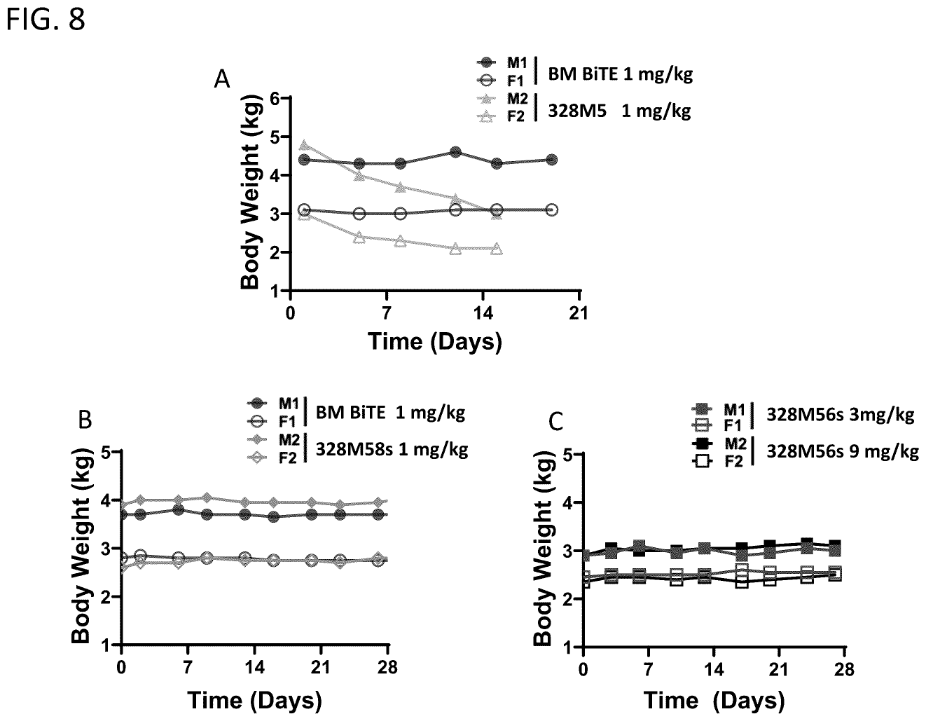

FIG. 8 shows body weight changes in cynomolgus monkey after treatment with T cell engagers. Panel A shows that the first-generation TriTE 328M5 caused significant body weight loss along with other clinical abnormities when administered at 1 mg/kg, while BM BiTE does not. Panel B and Panel C show that the second-generation TriTEs, 328M56s and 328M58s, do not cause body weight loss in Cynomolgus monkey. No obvious body weight change for 328M56s up to 9 mg/kg, and there were no obvious abnormal clinical observations.

### Example 5: Multi-Specific Molecules of with CD3-, CD28- and DLL3-Targeting Domains and Low-Medium CD3/CD28 Binding Affinity (Second Generation 328D TriTEs) Show Significant T-Cell Activation and Anti-Tumor Potency with Reduced Toxicity

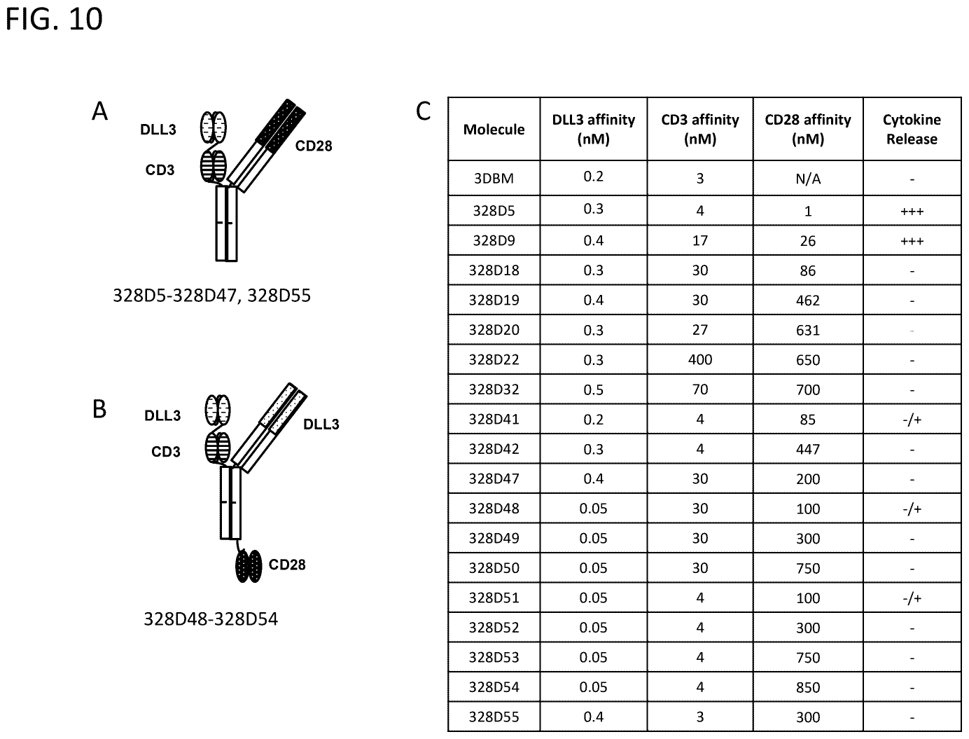

FIG. 9 depicts embodiments of multi-specific molecules of the 328D second generation TriTEs (FIG. 9, Panels A-I). FIG. 10, Panel C, shows target binding affinities and corresponding cytokine release of embodiments of benchmark molecules containing only CD3 and DLL3-targeting domains (e.g., 3DBM (tarlatamab)), multi-specific molecules containing CD3-, CD28- and DLL3-targeting domains with high affinity to CD3/CD28 (first generation 328D TriTEs, e.g., 328D5 and 328D9). FIG. 10, Panels A and B, show the second generation 328D TriTEs in two example configurations.

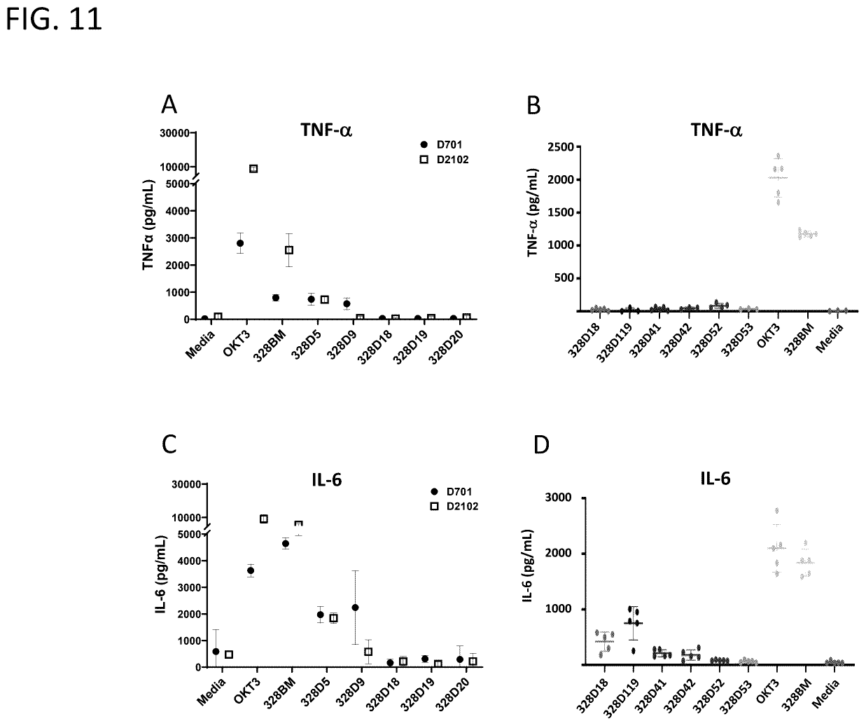

The second generation 328D TriTEs shows examples of reduced target independent cytokine secretions compared to benchmark molecules and the first generation 328D TriTE (FIG. 11, Panel A and B, release of TNFα, Panel C and D, release of IL-6). In contrast to OKT3, a clinical CD3×CD28 TriTE benchmark (SAR443216 shown as 328BM in Panels A-D), and first-generation TriTE 328D molecules (328D5 and 328D9), most of the second generation TriTE 328D molecules demonstrate a clean nonspecific cytokine profile in the cytokine release assay.

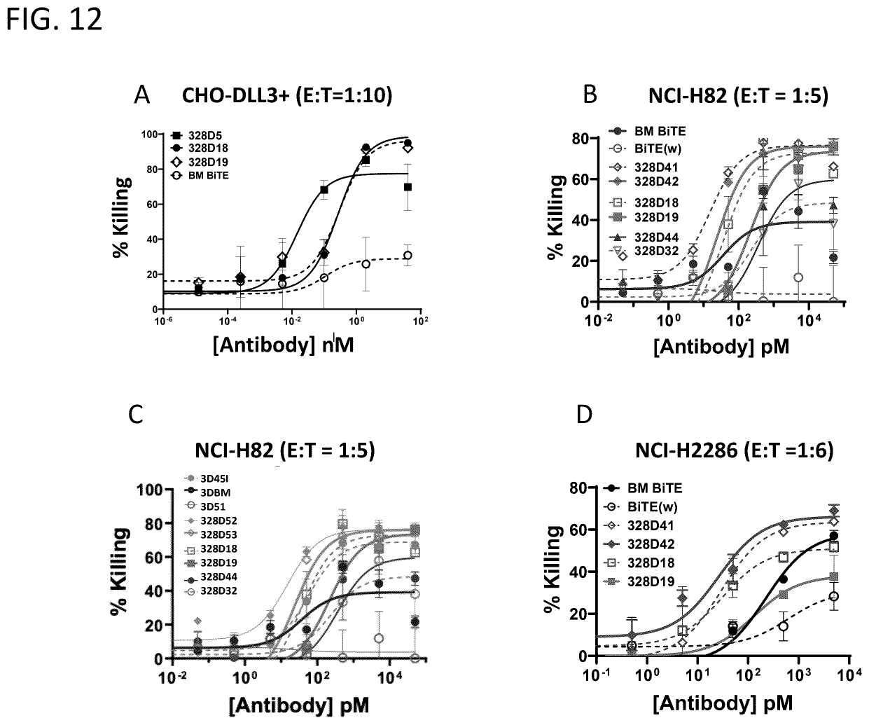

FIG. 12 shows examples of screening results from TDCC assay on various TriTE 328D molecules compared to BM BiTE and a reduced CD3 affinity BM BITE (BITEw). The assays were conducted on different target cells, including CHO-DLL3+ cells (Panel A), NCI-H82 (Panel B and C), and NCI-H2286 (Panel D), using low effector-target cell ratios and extended assay times up to 7 days. The second generation TriTE 328D molecules demonstrate superiorities over benchmark BiTEs in the TDCC assay. Multiple second generation TriTE have potent TDCC activities while exhibiting minimal cytokine release.

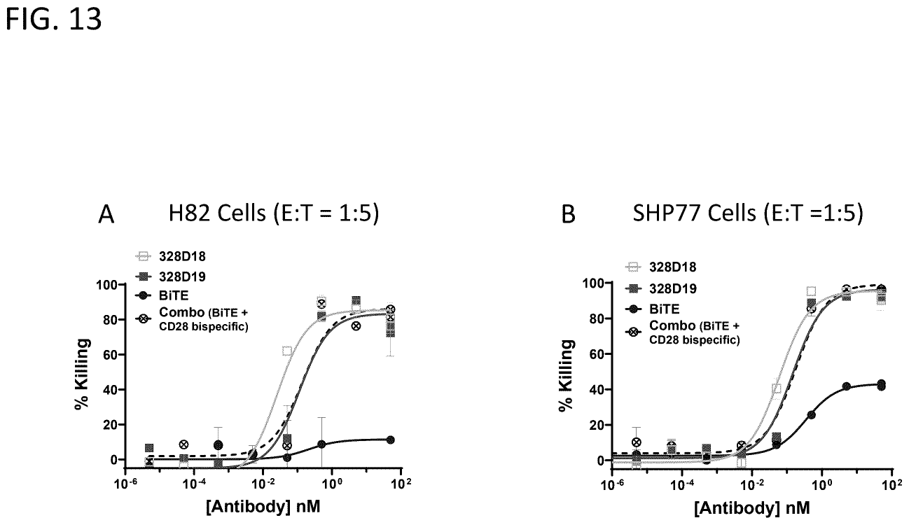

FIG. 13 shows comparative analysis of some second-generation 328D TriTE molecules, benchmark BITE (BiTEw) of similar weak CD3 Affinity, and combination treatment in TDCC assay in cancer cell line H82 (Panel A) and SHP77 (Panel B) using low effector-to-target ratio and extended assay times. The second generation 328D TriTEs demonstrated a significant superiority in TDCC activity compared to a BiTE with the same CD3 affinity. Additionally, the second generation TriTEs performed on par with a combination of two DLL3 bispecifics (DLL3×CD3 BITE+DLL3×CD28 bispecific), despite the CD28 affinity in combination being significantly higher at 2 nM, highlighting the advantage of the second generation TriTEs.

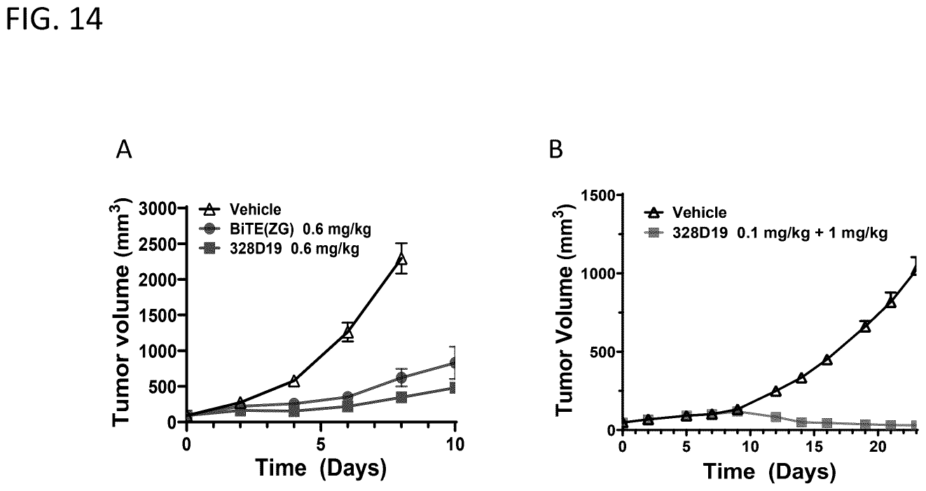

FIG. 14 shows efficacy of a 328D molecule on tumor growth in huCD3/huCD28 knock-in syngeneic mouse model bearing B16F10-hDLL3+ (Biocytogen CRO). Panel A: Effect of second generation 328D in huCD3/huCD28 mice bearing hDLL3-B16F10-hDLL3 cells. Panel B: Effect of second generation 328D in NOG mice bearing PBMC mixed with SHP-77 cells. TriTE demonstrated significant anti-tumor effect at low dose, superior to BM BiTE (ZG006, a clinical BiTE)

### Example 6: Multi-Specific Molecules of with CD3-, CD28- and CLDN-Targeting Domains and Low-Medium CD3/CD28 Binding Affinity (Second Generation 328C TriTEs) Show Significant T-Cell Activation and Anti-Tumor Potency with Reduced Toxicity

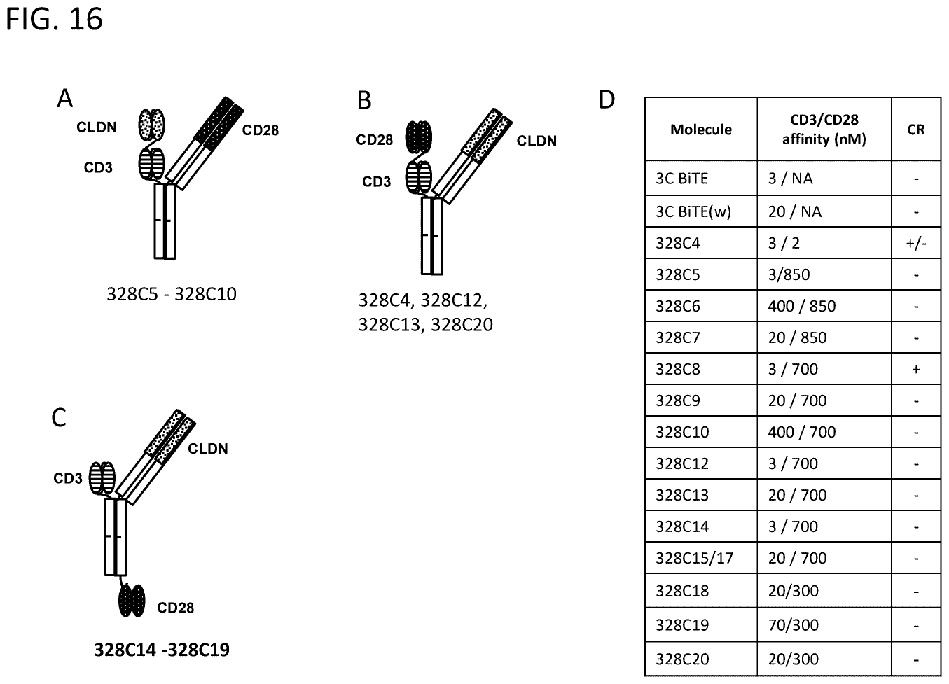

FIG. 15 depicts embodiments of multi-specific molecules of the 328C second generation TriTEs (Panels A-I). FIG. 16 shows multi-specific molecules containing CD3-, CD28- and CLDN-targeting domains with high affinity to CD3/CD28 (first generation 328C TriTEs, e.g., 328C4), and the second generation 328C TriTEs in three different configurations (Panels A-C). Panel D shows corresponding affinities and cytokine release of molecules depicted.

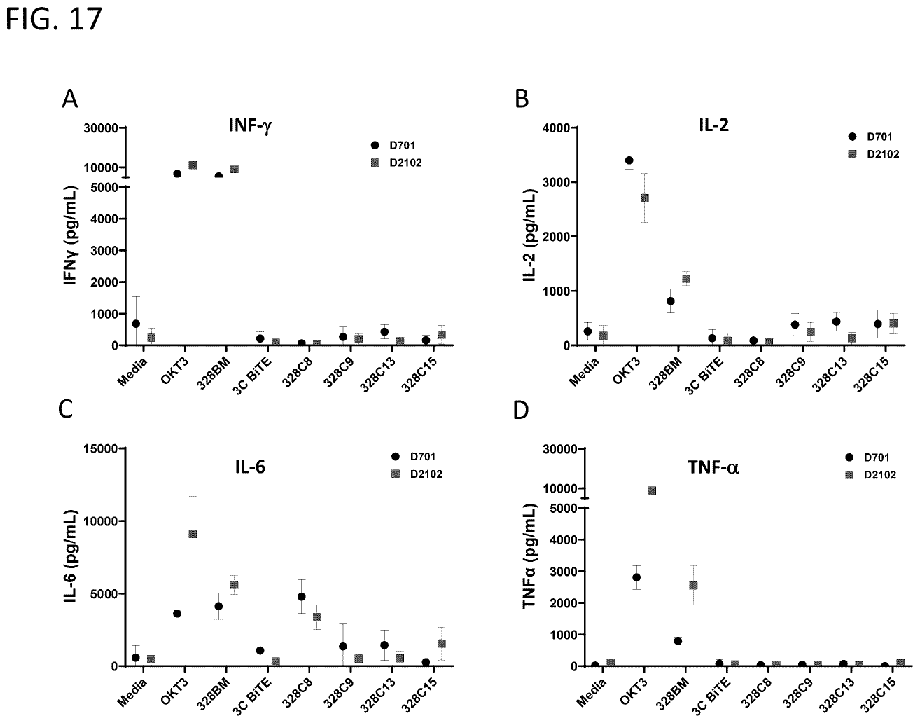

FIG. 17 shows sample results of cytokine release assays that assess nonspecific cytokine secretion from PBMC cell exposed to 328C TriTE molecules. Panel A, release of IFNγ. Panel B, release of IL2. Panel C, release of IL6. Panel D, release of TNFα. Most of the second generation 328C TriTEs demonstrates a clean nonspecific cytokine profile in the cytokine release assay in contrast to a benchmark TriTE (328BM: SAR443216).

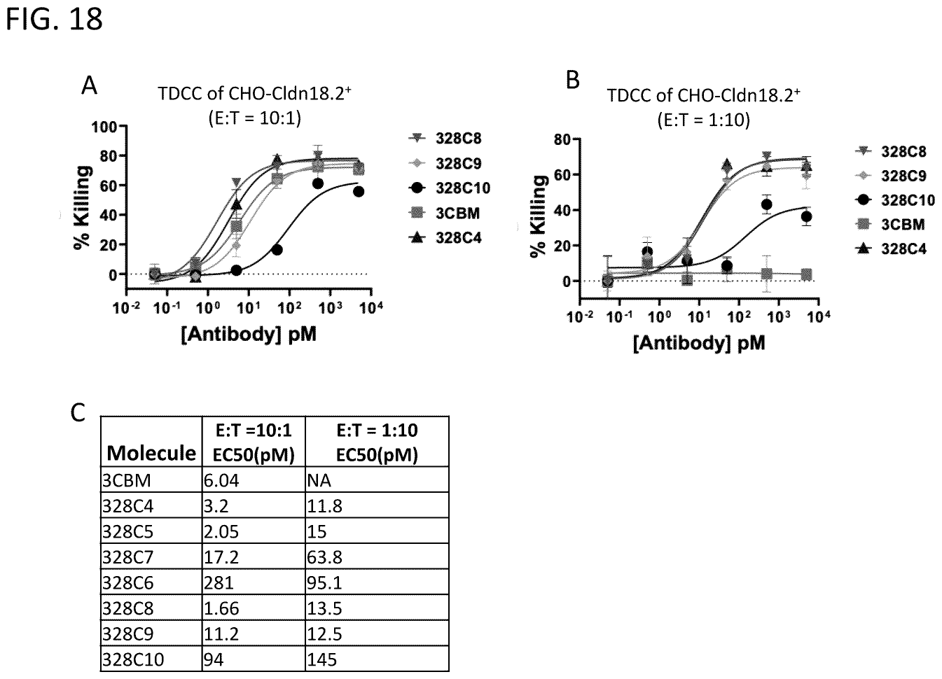

FIG. 18 shows an example results of TDCC assays that demonstrate second generation 328C TriTEs' superiority over corresponding BiTE molecule (3CBM). Panel A, CHO-CLDN+TDCC at effector-to-target ratio of 10:1 and assay time of 1 day. Panel B, CHO-CLDN killing at effector-to-target ratio of 1:10 and extended assay time of 5 days. Killing of 328C5-7 (CD28>800 nM) were also performed. Curves and potency are similar to 328C8-10 series (CD28_700). Panel C, EC50 of CHO-CLDN killing at high and low effector-to-target ratios.

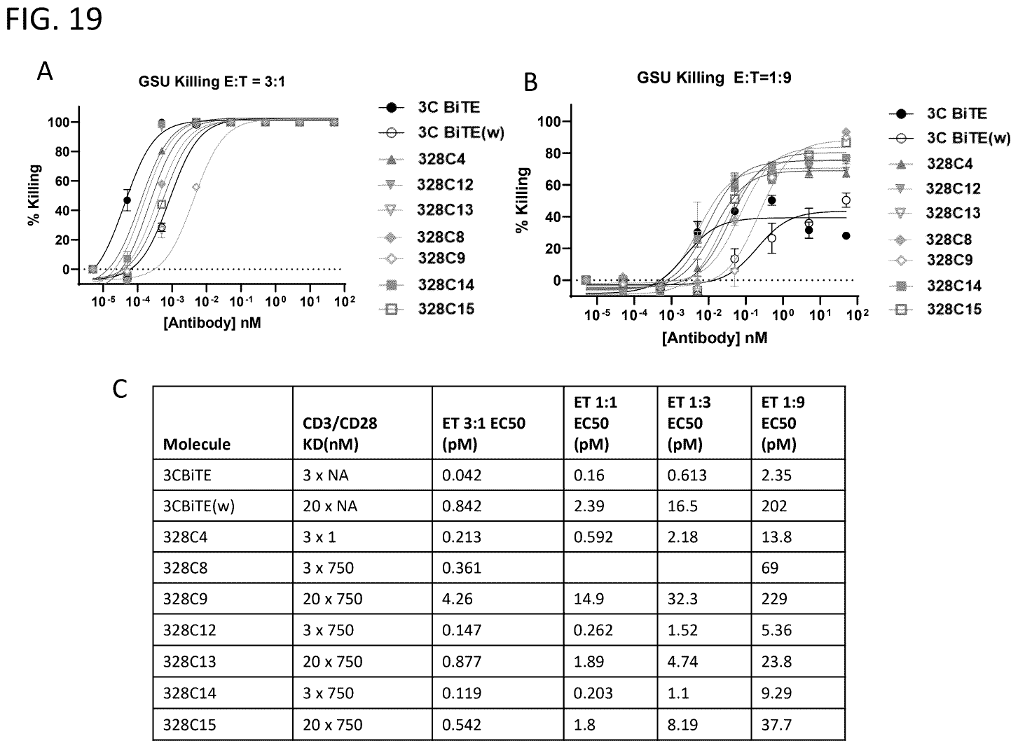

FIG. 19 shows another example results of TDCC assays using GSU cancer cell line that compare different TriTEs at different effector:target (E:T) ratios and assay times. Panel A, GSU killing at E:T=3:1. Panel B, GSU killing at E:T=1:9. The EC50 of GSU killing at different E:T ratios are shown Panel C.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 18, 2024, is named “2022-008.xml” and is 216,843 bytes in size. The sequence listing contained in this XML file is part of the specification and is hereby incorporated by reference herein in its entirety.

Sequences listed in application

| SEQ ID | | NAME |
|---|---|---|
| NO | SEQUENCE | |
| 1 | GYTFTSYW | hu1MU32A, hu1MU8A, |
| | | HCDR1 |
| 2 | IHPSDSET | hu1MU32A, HCDR2 |
| 3 | ARQGIITSVQEFAY | hu1MU32A, hu1MU8A, |
| | | HCDR3 |
| 4 | SSVNY | hu1MU32A, hu1MU8A, |
| | | LCDR1 |
| 5 | RTS | hu1MU32A, hu1MU11A, |
| | | hu1MU8A, LCDR2 |
| 6 | QQYHSYPLT | hu1MU32A, hu1MU8A, |
| | | LCDR3 |
| 7 | QVQLVQSGAEVKKPGSSVKVSCKASGYTFTSYWMNWVRQAPGQCLE | hu1MU32A, VH |
| | WMGMIHPSDSETRLNQKFKDRVTITADKSTSTAYMELSSLRSEDTAVYY | |
| | CARQGIITSVQEFAYWGQGTLVTVSS | |
| 8 | IQMTQSPSSLSASVGDRVTITCSASSSVNYIYWYQQKPGKAPKLLIYRTS | hu1MU32A, VL |
| | NLASGVPSRFSGSGSGTDFTLTISSLQPEDFATYYCQQYHSYPLTFGGG | |
| | TKVEIK | |
| 9 | GYEFSSHW | hu1MU11A, HCDR1 |
| 10 | IYPGDGDI | hu1MU11A, HCDR2 |
| 11 | ARHGNYVMDY | hu1MU11A, HCDR3 |
| 12 | SSVSY | hu1MU11A, LCDR1 |
| 13 | QQFHDYPRT | hu1MU11A, LCDR3 |
| 14 | EVQLVQSGAEVKKPGESLKISCKGSGYEFSSHWMNWVRQMPGKGLE | hu1MU11A, VH |
| | WMGQIYPGDGDINYNEKFRGQVTISADKSISTAYLQWSSLKASDTAMYY | |
| | CARHGNYVMDYWGQGTLVTVSS | |
| 15 | IQLTQSPSFLSASVGDRVTITCSASSSVSYMFWYQQKPGKAPKPWIYRT | hu1MU11A, VL |
| | SNLASGVPSRFSGSGSGTEFTLTISSLQPEDFATYYCQQFHDYPRTFGG | |
| | GTKVEIK | |
| 16 | IHPSDSES | hu1MU8A, HCDR 2 |
| 17 | QVQLVQSGAEVKKPGSSVKVSCKASGYTFTSYWLNWVRQAPGQGLE | hu1MU8A, VH |
| | WMGMIHPSDSESRLNQKFKDRVTITADKSTSTAYMELSSLRSEDTAVYY | |
| | CARQGIITSVQEFAYWGQGTLVTVSS | |
| 18 | IQMTQSPSSLSASVGDRVTITCSASSSVNYIFWYQQKPGKAPKLLIYRTS | hu1MU8A, VL |
| | NLASGVPSRFSGSGSGTDFTLTISSLQPEDFATYYCQQYHSYPLTFGGG | |
| | TKVEIK | |
| 19 | GFTFSSFG | hu1MU37, HCDR1 |
| 20 | ISSGSSTI | hu1MU37, HCDR2 |
| 21 | ARWGYYGSSYFAY | hu1MU37, HCDR3 |
| 22 | EDIYNR | hu1MU37, LCDR1 |
| 23 | GAT | hu1MU37, LCDR2 |
| 24 | QQFWRTPPT | hu1MU37, LCDR3 |
| 25 | EVQLVESGGGLVQPGGSLRLSCAASGFTFSSFGMHWVRQAPGKGLE | hu1MU37, VH |
| | WVSYISSGSSTIYYADTVKGRFTISRDNAKNSLYLQMNSLRAEDTAVYY | |
| | CARWGYYGSSYFAYWGQGTLVTVSS | |
| 26 | DIQMTQSPSSLSASVGDRVTITCKASEDIYNRLAWYQQKPGKAPKPLIS | hu1MU37, VL |
| | GATNLETGVPSRFSGSGSGTDFTFTISSLQPEDIATYYCQQFWRTPPTF | |
| | GGGTKVEIK | |
| 27 | GGSFSGYY | HuS_anti-Muc17, HCDR1 |
| 28 | IDASGST | HuS_anti-Muc17, HCDR2 |
| 29 | ARKKYSTVWSYFDN | HuS_anti-Muc17, HCDR3 |
| 30 | KLGDKY | HuS_anti-Muc17, LCDR1 |
| 31 | QDR | HuS_anti-Muc17, LCDR2 |
| 32 | QAWGSSTAV | HuS_anti-Muc17, LCDR3 |
| 33 | QVQLQQWGAGLLKPSETLSLTCAVYGGSFSGYYWSWIRQPPGKCLEW | HuS_anti-Muc17, VH |
| | IGDIDASGSTKYNPSLKSRVTISLDTSKNQFSLKLNSVTAADTAVYFCAR | |
| | KKYSTVWSYFDNWGQGTLVTVSS | |
| 34 | SYELTQPSSVSVPPGQTASITCSGDKLGDKYASWYQQKPGQSPVLVIY | HuS_anti-Muc17, VL |
| | QDRKRPSGVPERFSGSNSGNTATLTISGTQAMDEADYYCQAWGSSTA | |
| | VFGCGTKLTVL | |
| 35 | GYTFTDYF | huD139A, HCDR1 |
| 36 | INPYNDIT | huD139A, HCDR2 |
| 37 | AREGVLYDGYYEGAY | huD139A, HCDR3 |
| 38 | QNVGIA | huD139A, LCDR1 |
| 39 | AAS | huD139A, LCDR2 |
| 40 | QQYSTYPYT | huD139A, LCDR3 |
| 41 | QVQLVQSGAEVKKPGASVKVSCKASGYTFTDYFMNWVRQAPGQCLE | huD139A, VH |
| | WMGVINPYNDITIYNQKFQGRVTMTVDRSTSTVYMELSSLRSEDTAVYY | |
| | CAREGVLYDGYYEGAYWGQGTLVTVSS | |
| 42 | DIQLTQSPSFLSASVGDRVTITCKASQNVGIAVAWYQQKPGKAPKLLIYA | huD139A, VL |
| | ASNRYTGVPSRFSGSGSGTEFTLTISSLQPEDFATYYCQQYSTYPYTFG | |
| | CGTKLEIK | |
| 43 | GFTFSSYG | hu1D4, HCDR1 |
| 44 | ISHHGSSK | hu1D4, HCDR2 |
| 45 | ARDWFFYLFDY | hu1D4, HCDR3 |
| 46 | QSLLHSDAKTF | hu1D4, LCDR1 |
| 47 | EVS | hu1D4, LCDR2 |
| 48 | LQGERLPFT | hu1D4, LCDR3 |
| 49 | EVQLLESGGGLVQPGGSLRLSCAASGFTFSSYGMHWVRQAPGKGLEW | hu1D4, VH |
| | VAVISHHGSSKYYADSVKGRFTISRDNSKNTLYLQMNSLRAEDTAVYYC | |
| | ARDWFFYLFDYWGQGTLVTVSS | |
| 50 | DIQMTQSPSSLSASVGDRVTITCKSSQSLLHSDAKTFLYWYQQKPGKAP | hu1D4, VL |
| | KLLIYEVSNRFSGVPSRFSGSGSGTDFTLTISSLQPEDFATYYCLQGERL | |
| | PFTFGQGTKVEIK | |
| 51 | DYIFSNYY | huD143, HCDR1 |
| 52 | ILPGTGNT | huD143, HCDR2 |
| 53 | ARWGDYALFAN | huD143, huD138C, HCDR3 |
| 54 | QNVGTN | huD143, huD138C, LCDR1 |
| 55 | STS | huD143, LCDR2 |
| 56 | QQYNNYPLT | huD143, LCDR3 |
| 57 | QVQLVQSGAEVKKPGASVKVSCKATDYIFSNYYIEWVRQAPGQGLEW | huD143, VH |
| | MGEILPGTGNTVYNEKFKDRVTMTVDTSTSTVYMELSSLRSEDTAVYYC | |
| | ARWGDYALFANWGQGTLVTVSS | |
| 58 | DIQMTQSPSFLSASVGDRVTITCKASQNVGTNVAWYQQKPGKAPKPLIY | huD143, VL |
| | STSYRYSGVPSRFSGSGSGTEFTLTISSLQPEDFATYYCQQYNNYPLTF | |
| | GGGTKVEIK | |
| 59 | DYTFSNYY | huD138C, HCDR1 |
| 60 | ILPGNGNT | huD138C, HCDR2 |
| 61 | SAS | huD138C, LCDR2 |
| 62 | QQYNSYPFT | huD138C, LCDR3 |
| 63 | QVQLVQSGAEVKKPGASVKVSCKASDYTFSNYYIEWVRQAPGQGLEW | huD138C, VH |
| | MGEILPGNGNTVYNEKFKDRVTMTVDTSTSTAYMELRSLRSDDTAVYY | |
| | CARWGDYALFANWGQGTLVTVSS | |
| 64 | DIQMTQSPSTLSASVGDRVTITCKASQNVGTNVAWYQQKPGKAPKALIY | huD138C, VL |
| | SASYRYSGVPSRFSGSGSGTEFTLTISSLQPDDFATYYCQQYNSYPFTF | |
| | GQGTKLEIK | |
| 65 | GYTFTNSG | huCldn29B, HCDR1 |
| 66 | INTNTGEP | huCldn29B, HCDR2 |
| 67 | ARYYYGNSFAY | huCldn29B, HCDR3 |
| 68 | QSLLNSGNQKNY | huCldn29B, huCldn20, |
| | | LCDR1 |
| 69 | WAS | huCldn29B, huCldn20, |
| | | huCldn16A, huCldn23A, |
| | | LCDR2 |
| 70 | QNNYFYPLT | huCldn29B, LCDR3 |
| 71 | QVQLVQSGAEVKKPGASVKVSCKASGYTFTNSGMNWVRQAPGQGLE | huCldn29B, VH |
| | WMGWINTNTGEPTFAEEFRGRVTMTRDTSISTAYMELSRLRSDDTAVY | |
| | YCARYYYGNSFAYWGQGTLVTVSS | |
| 72 | DIVMTQSPDSLAVSLGERATINCKSSQSLLNSGNQKNYLTWYQQKPGQ | huCldn29B, VL |
| | PPKLLIYWASTRESGVPDRFSGSGSGTDFTLTISSLQAEDVAVYYCQNN | |
| | YFYPLTFGGGTKVEIK | |
| 73 | GYTFTNFG | huCldn16A, HCDR1 |
| 74 | IYPSSGNT | huCldn16A, HCDR2 |
| 75 | ARGGGPLRSRYFDY | huCldn16A, HCDR3 |
| 76 | QSLFSSGNQKNY | huCldn16A, LCDR1 |
| 77 | QNDYYYPLT | huCldn16A, LCDR3 |
| 78 | QVQLVQSGAEVKKPGASVKVSCKASGYTFTNFGITWVRQAPGQGLEW | huCldn16A, VH |
| | MGEIYPSSGNTFYNEKFKGRVTMTTDTSTSTAYMELRSLRSDDTAVYY | |
| | CARGGGPLRSRYFDYWGQGTLVTVSS | |
| 79 | DIVMTQSPDSLAVSLGERATINCRSSQSLFSSGNQKNYLTWYQQKPGQ | huCldn 16A, VL |
| | PPKLLIYWASTRESGVPDRFSGSGSGTDFTLTISSLQAEDVAVYYCQND | |
| | YYYPLTFGGGTKVEIK | |
| 80 | GFTFSSFG | huCldn20, HCDR1 |
| 81 | ISSGNSAI | huCldn20, HCDR2 |
| 82 | ARLRYGNSFDY | huCldn20, HCDR3 |
| 83 | QNNYYYPLT | huCldn20, LCDR3 |
| 84 | EVQLVESGGGLVQPGGSLRLSCAASGFTFSSFGMHWVRQAPGKGLE | huCldn20, VH |
| | WVSYISSGNSAIYYADTVNGRFTISRDNAKNSLYLQMNSLRAEDTAVYY | |
| | CARLRYGNSFDYWGQGTLVTVSS | |
| 85 | DIVMTQSPDSLAVSLGERATINCKSSQSLLNSGNQKNYLTWYQQKPGQ | huCldn20, VL |
| | PPKLLIYWASTRESGVPDRFSGSGSGTDFTLTISSLQAEDVAVYYCQNN | |
| | YYYPLTFGGGTKVEIK | |
| 86 | GYAFNNYW | huCldn23A, HCDR1 |
| 87 | ISPGNGNS | huCldn23A, HCDR2 |
| 88 | ARGGRYGNAMDY | huCldn23A, HCDR3 |
| 89 | QSLLNSGNQRNY | huCldn23A, LCDR1 |
| 90 | QNAYFYPYT | huCldn23A, LCDR3 |
| 91 | QVQLVQSGAEVKKPGSSVKVSCKASGYAFNNYWMNWVRQAPGQGLE | huCldn23A, VH |
| | WMGQISPGNGNSNFNGKFKGRVTITADKSTSTAYMELSSLRSEDTAVY | |
| | YCARGGRYGNAMDYWGQGTTVTVSS | |
| 92 | DIVMTQSPDSLAVSLGERATINCKSSQSLLNSGNQRNYLTWYQQKPGQ | huCldn23A, VL |
| | PPKLLIYWASTRESGVPDRFSGSGSGTDFTLTISSLQAEDVAVYYCQNA | |
| | YFYPYTFGGGTKVEIK | |
| 93 | GFTFSTYA | aCD3 (VH_A116E), |
| | | aCD3 (VH_A116E, Y60A), |
| | | aCD3 (VH_A116E, K59E, |
| | | Y60A), |
| | | aCD3 (VH_A116D), |
| | | HCDR1 |
| 94 | IRSKYNNYAT | aCD3 (VH_A116E), |
| | | aCD3 (VH_A116D), |
| | | HCDR2 |
| 95 | VRHGNFGDSYVSWFEY | aCD3 (VH_A116E), |
| | | aCD3 (VH_A116E, Y60A), |
| | | aCD3 (VH_A116E, K59E, |
| | | Y60A), |
| | | HCDR3 |
| 96 | TGAVTTSNY | aCD3 (VH_A116E), |
| | | aCD3 (VH_A116E, Y60A), |
| | | aCD3 (VH_A116E, K59E, |
| | | Y60A), |
| | | aCD3 (VH_A116D), |
| | | LCDR1 |
| 97 | GTN | aCD3 (VH_A116E), |
| | | aCD3 (VH_A116E, Y60A), |
| | | aCD3 (VH_A116E, K59E, |
| | | Y60A) |
| | | aCD3 (VH_A116D), |
| | | LCDR2 |
| 98 | ALWYSNHWV | aCD3 (VH_A116E), |
| | | aCD3 (VH_A116E, Y60A), |
| | | aCD3 (VH_A116E, K59E, |
| | | Y60A), |
| | | aCD3 (VH_A116D), |
| | | LCDR3 |
| 99 | EVQLVESGGGLVQPGGSLRLSCAASGFTFSTYAMNWVRQAPGKGLEW | aCD3 (VH_A116E), VH |
| | VGRIRSKYNNYATYYADSVKGRFTISRDDSKNTLYLQMNSLRAEDTAVY | |
| | YCVRHGNFGDSYVSWFEYWGQGTLVTVSS | |
| 100 | QAVVTQEPSLTVSPGGTVTLTCGSSTGAVTTSNYANWVQQKPGKSPR | aCD3 (VH_A116E), VL |
| | GLIGGTNKRAPGVPARFSGSLLGGKAALTISGAQPEDEADYYCALWYS | aCD3 (VH_A116E, Y60A), |
| | NHWVFGGGTKLTVL | VL |
| | | aCD3 (VH_A116E, K59E, |
| | | Y60A), VL |
| | | aCD3 (VH_A116D), VL |
| 101 | IRSKANNYAT | aCD3 (VH_A116E, Y60A), |
| | | HCDR2 |
| 102 | EVQLVESGGGLVQPGGSLRLSCAASGFTFSTYAMNWVRQAPGKGLEW | aCD3 (VH_A116E, Y60A), |
| | VGRIRSKANNYATYYADSVKGRFTISRDDSKNTLYLQMNSLRAEDTAVY | VH |
| | YCVRHGNFGDSYVSWFEYWGQGTLVTVSS | |
| 103 | IRSEANNYAT | aCD3 (VH_A116E, K59E, |
| | | Y60A), HCDR2 |
| 104 | EVQLVESGGGLVQPGGSLRLSCAASGFTFSTYAMNWVRQAPGKGLEW | aCD3 (VH_A116E, K59E, |
| | VGRIRSEANNYATYYADSVKGRFTISRDDSKNTLYLQMNSLRAEDTAVY | Y60A), VH |
| | YCVRHGNFGDSYVSWFEYWGQGTLVTVSS | |
| 105 | VRHGNFGDSYVSWFDY | aCD3 (VH_A116D), HCDR3 |
| 106 | EVQLVESGGGLVQPGGSLRLSCAASGFTFSTYAMNWVRQAPGKGLEW | aCD3 (VH_A116D), VH |
| | VGRIRSKYNNYATYYADSVKGRFTISRDDSKNTLYLQMNSLRAEDTAVY | |
| | YCVRHGNFGDSYVSWFDYWGQGTLVTVSS | |
| 107 | GYTFTSYY | aCD28 (VH_C55S), |
| | | aCD28 (VH_C55S, Y109F, |
| | | L111Y), |
| | | aCD28 (VH_C55S, N62S, |
| | | L111A), |
| | | aCD28 (VH_C55S, N62S, |
| | | Y109F, L111Y), |
| | | aCD28 (VH_C55S, L111Y, |
| | | W113F), |
| | | aCD28 (VH_C55S, W113A), |
| | | HCDR1 |
| 108 | IYPGNVNT | aCD28 (VH_C55S), |
| | | aCD28 (VH_C55S, Y109F, |
| | | L111Y), |
| | | aCD28 (VH_C55S, S36G, |
| | | L111A), |
| | | aCD28 (VH_C55S, S36G, |
| | | Y109F, L111Y), |
| | | aCD28 (VH_C55S, L111Y, |
| | | W113F), |
| | | aCD28 (VH_C55S, W113A), |
| | | HCDR2 |
| 109 | TRSHYGLDWNFDV | aCD28 (VH_C55S), HCDR3 |
| 110 | QNIYVW | aCD28 (VH_C55S), |
| | | aCD28 (VH_C55S, Y109F, |
| | | L111Y), |
| | | aCD28 (VH_C55S, S36G, |
| | | L111A), |
| | | aCD28 (VH_C55S, N62S, |
| | | L111A), |
| | | aCD28 (VH_C55S, S36G, |
| | | Y109F, L111Y), |
| | | aCD28 (VH_C55S, N62S, |
| | | Y109F, L111Y), |
| | | aCD28 (VH_C55S, L111Y, |
| | | W113F), |
| | | aCD28 (VH_C55S, W113A), |
| | | LCDR1 |
| 111 | KAS | aCD28 (VH_C55S), |
| | | aCD28 (VH_C55S, Y109F, |
| | | L111Y), |
| | | aCD28 (VH_C55S, S36G, |
| | | L111A), |
| | | aCD28 (VH_C55S, N62S, |
| | | L111A), |
| | | aCD28 (VH_C55S, S36G, |
| | | Y109F, L111Y), |
| | | aCD28 (VH_C55S, N62S, |
| | | Y109F, L111Y), |
| | | aCD28 (VH_C55S, L111Y, |
| | | W113F), |
| | | aCD28 (VH_C55S, W113A), |
| | | LCDR2 |
| 112 | QQGQTYPYT | aCD28 (VH_C55S), |
| | | aCD28 (VH_C55S, Y109F, |
| | | L111Y), |
| | | aCD28 (VH_C55S, S36G |
| | | L111A), |
| | | aCD28 (VH_C55S, N62S, |
| | | L111A), |
| | | aCD28 (VH_C55S, S36G, |
| | | Y109F, L111Y), |
| | | aCD28 (VH_C55S, N62S, |
| | | Y109F, L111Y), |
| | | aCD28 (VH_C55S, L111Y, |
| | | W113F), |
| | | aCD28 (VH_C55S, W113A), |
| | | LCDR3 |
| 113 | QVQLVQSGAEVKKPGASVKVSCKASGYTFTSYYIHWVRQAPGQGLEWI | aCD28 (VH_C55S), VH |
| | GSIYPGNVNTNYNEKFKDRATLTVDTSISTAYMELSRLRSDDTAVYFCT | |
| | RSHYGLDWNFDVWGQGTTVTVSS | |
| 114 | DIQMTQSPSSLSASVGDRVTITCHASQNIYVWLNWYQQKPGKAPKLLIY | aCD28 (VH_C55S), VL |
| | KASNLHTGVPSRFSGSGSGTDFTLTISSLQPEDFATYYCQQGQTYPYTF | aCD28 (VH_C55S, Y109F, |
| | GGGTKVEIK | L111Y), VL |
| | | aCD28 (VH_C55S, S36G, |
| | | L111A), VL |
| | | aCD28 (VH_C55S, N62S, |
| | | L111A), VL |
| | | aCD28 (VH_C55S, S36G, |
| | | Y109F, L111Y), VL |
| | | aCD28 (VH_C55S, N62S, |
| | | Y109F, L111Y), VL |
| | | aCD28 (VH_C55S, L111Y, |
| | | W113F), VL |
| | | aCD28 (VH_C55S, W113A), |
| | | VL |
| 115 | TRSHFGYDWNFDV | aCD28 (VH_C55S, Y109F, |
| | | L111Y), |
| | | aCD28 (VH_C55S, S36G, |
| | | Y109F, L111Y), |
| | | aCD28 (VH_C55S, N62S, |
| | | Y109F, L111Y), |
| | | HCDR3 |
| 116 | QVQLVQSGAEVKKPGASVKVSCKASGYTFTSYYIHWVRQAPGQGLEWI | aCD28 (VH_C55S, Y109F, |
| | GSIYPGNVNTNYNEKFKDRATLTVDTSISTAYMELSRLRSDDTAVYFCT | L111Y), VH |
| | RSHFGYDWNFDVWGQGTTVTVSS | |
| 117 | GYTFTGYY | aCD28 (VH_C55S, S36G, |
| | | L111A), |
| | | aCD28 (VH_C55S, S36G, |
| | | Y109F, L111Y), |
| | | HCDR1 |
| 118 | TRSHYGADWNFDV | aCD28 (VH_C55S, S36G, |
| | | L111A) |
| | | aCD28 (VH_C55S, N62S, |
| | | L111A), |
| | | HCDR3 |
| 119 | QVQLVQSGAEVKKPGASVKVSCKASGYTFTGYYIHWVRQAPGQGLEWI | aCD28 (VH_C55S, S36G, |
| | GSIYPGNVNTNYNEKFKDRATLTVDTSISTAYMELSRLRSDDTAVYFCT | L111A), VH |
| | RSHYGADWNFDVWGQGTTVTVSS | |
| 120 | IYPGSVNT | aCD28 (VH_C55S, N62S, |
| | | L111A), |
| | | aCD28 (VH_C55S, N62S, |
| | | Y109F, L111Y), HCDR2 |
| 121 | QVQLVQSGAEVKKPGASVKVSCKASGYTFTSYYIHWVRQAPGQGLEWI | aCD28 (VH_C55S, N62S, |
| | GSIYPGSVNTNYNEKFKDRATLTVDTSISTAYMELSRLRSDDTAVYFCTR | L111A), VH |
| | SHYGADWNFDVWGQGTTVTVSS | |
| 122 | QVQLVQSGAEVKKPGASVKVSCKASGYTFTGYYIHWVRQAPGQGLEWI | aCD28 (VH_C55S, S36G, |
| | GSIYPGNVNTNYNEKFKDRATLTVDTSISTAYMELSRLRSDDTAVYFCT | Y109F, L111Y), VH |
| | RSHFGYDWNFDVWGQGTTVTVSS | |
| 123 | QVQLVQSGAEVKKPGASVKVSCKASGYTFTSYYIHWVRQAPGQGLEWI | aCD28 (VH_C55S, N62S, |
| | GSIYPGSVNTNYNEKFKDRATLTVDTSISTAYMELSRLRSDDTAVYFCTR | Y109F, L111Y), VH |
| | SHFGYDWNFDVWGQGTTVTVSS | |
| 124 | TRSHYGYDFNFDV | aCD28 (VH_C55S, L111Y, |
| | | W113F), HCDR3 |
| 125 | QVQLVQSGAEVKKPGASVKVSCKASGYTFTSYYIHWVRQAPGQGLEWI | aCD28 (VH_C55S, L111Y, |
| | GSIYPGNVNTNYNEKFKDRATLTVDTSISTAYMELSRLRSDDTAVYFCT | W113F), VH |
| | RSHYGYDFNFDVWGQGTTVTVSS | |
| 126 | TRSHYGLDANFDV | aCD28 (VH_C55S, W113A), |
| | | HCDR3 |
| 127 | QVQLVQSGAEVKKPGASVKVSCKASGYTFTSYYIHWVRQAPGQGLEWI | aCD28 (VH_C55S, W113A), |
| | GSIYPGNVNTNYNEKFKDRATLTVDTSISTAYMELSRLRSDDTAVYFCT | VH |
| | RSHYGLDANFDVWGQGTTVTVSS | |
| 128 | QVQLVQSGAEVKKPGSSVKVSCKASGYTFTSYWMNWVRQAPGQCLE | 328M55 (chain 1) |
| | WMGMIHPSDSETRLNQKFKDRVTITADKSTSTAYMELSSLRSEDTAVYY | |
| | CARQGIITSVQEFAYWGQGTLVTVSSGGGGSGGGGSGGGGSGGGGSI | |
| | QMTQSPSSLSASVGDRVTITCSASSSVNYIYWYQQKPGKAPKLLIYRTS | |
| | NLASGVPSRFSGSGSGTDFTLTISSLQPEDFATYYCQQYHSYPLTFGCG | |
| | TKVEIKSGGGGSEVQLVESGGGLVQPGGSLRLSCAASGFTFSTYAMN | |
| | WVRQAPGKGLEWVGRIRSKYNNYATYYADSVKGRFTISRDDSKNTLYL | |
| | QMNSLRAEDTAVYYCVRHGNFGDSYVSWFEYWGQGTLVTVSSGGGG | |
| | SGGGGSGGGGSQAVVTQEPSLTVSPGGTVTLTCGSSTGAVTTSNYAN | |
| | WVQQKPGKSPRGLIGGTNKRAPGVPARFSGSLLGGKAALTISGAQPED | |
| | EADYYCALWYSNHWVFGGGTKLTVLEPKSSDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALGAPIE | |
| | KTISKAKGQPREPQVYTLPPCRKKLTKNQVSLTCLVKGFYPSDIAVEWE | |
| | SNGQPENNYKTTPPVLKSDGSFFLYSKLTVDKSRWQQGNVFSCSVMH | |
| | EALHNHYTQKSLSLSPGK* | |
| 129 | QVQLVQSGAEVKKPGASVKVSCKASGYTFTSYYIHWVRQAPGQGLEWI | 328M55 (chain 2) |
| | GSIYPGNVNTNYNEKFKDRATLTVDTSISTAYMELSRLRSDDTAVYFCT | 328M56 (chain 2) |
| | RSHYGYDFNFDVWGQGTTVTVSSASTKGPSVFPLAPSSKSTSGGTAAL | 328M56s (chain 2) |
| | GCLVKDYFPEPVTVSWNSGALTSGVHTFPAVLQSSGLYSLSSVVTVPS | 328M58 (chain 2) |
| | SSLGTQTYICNVNHKPSNTKVDKRVEPKSCDKTHTCPPCPAPEAAGGP | 328M58s (chain 2) |
| | SVFLFPPKPKDTLMISRTPEVTCVVVDVSHEDPEVKFNWYVDGVEVHN | 328D19 (chain 2) |
| | AKTKPREEQYNSTYRVVSVLTVLHQDWLNGKEYKCKVSNKALGAPIEKT | 328C8 (chain 2) |
| | ISKAKGQPREPQVCTLPPSRDELTKNQVSLTCLVEGFYPSDIAVEWESN | 328C9 (chain 2) |
| | GQPENNYKTTPPVLDSDGSFFLYSDLTVDKSRWQQGNVFSCSVMHEA | 328D42 (chain 2) |
| | LHNHYTQESLSLSPGK* | |
| 130 | DIQMTQSPSSLSASVGDRVTITCHASQNIYVWLNWYQQKPGKAPKLLIY | 328M32 (chain 3) |
| | KASNLHTGVPSRFSGSGSGTDFTLTISSLQPEDFATYYCQQGQTYPYTF | 328M55 (chain 3) |
| | GGGTKVEIKRTVAAPSVFIFPPSDEQLKSGTASVVCLLNNFYPREAKVQ | 328M56 (chain 3) |
| | WKVDNALQSGNSQESVTEQDSKDSTYSLSSTLTLSKADYEKHKVYACE | 328M56s (chain 3) |
| | VTHQGLSSPVTKSFNRGEC* | 328M57 (chain 3) |
| | | 328M58 (chain 3) |
| | | 328M58s (chain 3) |
| | | 328M59 (chain 3) |
| | | 328M62 (chain 3) |
| | | 328D18 (chain 3) |
| | | 328D19 (chain 3) |
| | | 328D20 (chain 3) |
| | | 328D41 (chain 3) |
| | | 328D42 (chain 3) |
| | | 328D47 (chain 3) |
| | | 328D55 (chain 3) |
| | | 328C8 (chain 3) |
| | | 328C9 (chain 3) |
| 131 | QVQLVQSGAEVKKPGSSVKVSCKASGYTFTSYWMNWVRQAPGQCLE | 328M56 (chain 1) |
| | WMGMIHPSDSETRLNQKFKDRVTITADKSTSTAYMELSSLRSEDTAVYY | 328M57 (chain 1) |
| | CARQGIITSVQEFAYWGQGTLVTVSSGGGGSGGGGGGGGSGGGGSI | 328M62 (chain 1) |
| | QMTQSPSSLSASVGDRVTITCSASSSVNYIYWYQQKPGKAPKLLIYRTS | |
| | NLASGVPSRFSGSGSGTDFTLTISSLQPEDFATYYCQQYHSYPLTFGCG | |
| | TKVEIKSGGGGSEVQLVESGGGLVQPGGSLRLSCAASGFTFSTYAMN | |
| | WVRQAPGKGLEWVGRIRSKANNYATYYADSVKGRFTISRDDSKNTLYL | |
| | QMNSLRAEDTAVYYCVRHGNFGDSYVSWFEYWGQGTLVTVSSGGGG | |
| | SGGGGSGGGGSQAVVTQEPSLTVSPGGTVTLTCGSSTGAVTTSNYAN | |
| | WVQQKPGKSPRGLIGGTNKRAPGVPARFSGSLLGGKAALTISGAQPED | |
| | EADYYCALWYSNHWVFGGGTKLTVLEPKSSDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALGAPIE | |
| | KTISKAKGQPREPQVYTLPPCRKKLTKNQVSLTCLVKGFYPSDIAVEWE | |
| | SNGQPENNYKTTPPVLKSDGSFFLYSKLTVDKSRWQQGNVFSCSVMH | |
| | EALHNHYTQKSLSLSPGK* | |
| 132 | QVQLVQSGAEVKKPGASVKVSCKASGYTFTSYYIHWVRQAPGQGLEWI | 328M32 (chain 2) |
| | GSIYPGNVNTNYNEKFKDRATLTVDTSISTAYMELSRLRSDDTAVYFCT | 328M57 (chain 2) |
| | RSHYGLDANFDVWGQGTTVTVSSASTKGPSVFPLAPSSKSTSGGTAAL | 328M59 (chain 2) |
| | GCLVKDYFPEPVTVSWNSGALTSGVHTFPAVLQSSGLYSLSSVVTVPS | 328D20 (chain 2) |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGK* | |
| 133 | QVQLVQSGAEVKKPGSSVKVSCKASGYTFTSYWMNWVRQAPGQCLE | 328M58 (chain 1) |
| | WMGMIHPSDSETRLNQKFKDRVTITADKSTSTAYMELSSLRSEDTAVYY | 328M59 (chain 1) |
| | CARQGIITSVQEFAYWGQGTLVTVSSGGGGSGGGGSGGGGSGGGGSI | |
| | QMTQSPSSLSASVGDRVTITCSASSSVNYIYWYQQKPGKAPKLLIYRTS | |
| | NLASGVPSRFSGSGSGTDFTLTISSLQPEDFATYYCQQYHSYPLTFGCG | |
| | TKVEIKSGGGGSEVQLVESGGGLVQPGGSLRLSCAASGFTFSTYAMN | |
| | WVRQAPGKGLEWVGRIRSKYNNYATYYADSVKGRFTISRDDSKNTLYL | |
| | QMNSLRAEDTAVYYCVRHGNFGDSYVSWFDYWGQGTLVTVSSGGGG | |
| | SGGGGSGGGGSQAVVTQEPSLTVSPGGTVTLTCGSSTGAVTTSNYAN | |
| | WVQQKPGKSPRGLIGGTNKRAPGVPARFSGSLLGGKAALTISGAQPED | |
| | EADYYCALWYSNHWVFGGGTKLTVLEPKSSDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALGAPIE | |
| | KTISKAKGQPREPQVYTLPPCRKKLTKNQVSLTCLVKGFYPSDIAVEWE | |
| | SNGQPENNYKTTPPVLKSDGSFFLYSKLTVDKSRWQQGNVFSCSVMH | |
| | EALHNHYTQKSLSLSPGK* | |
| 134 | QVQLVQSGAEVKKPGASVKVSCKASGYTFTDYFMNWVRQAPGQCLE | 328D18 (chain 1) |
| | WMGVINPYNDITIYNQKFQGRVTMTVDRSTSTVYMELSSLRSEDTAVYY | 328D19 (chain 1) |
| | CAREGVLYDGYYEGAYWGQGTLVTVSSGGGGSGGGGSGGGGSDIQL | 328D20 (chain 1) |
| | TQSPSFLSASVGDRVTITCKASQNVGIAVAWYQQKPGKAPKLLIYAASN | 328D47 (chain 1) |
| | RYTGVPSRFSGSGSGTEFTLTISSLQPEDFATYYCQQYSTYPYTFGCGT | 328D48 (chain 1) |
| | KLEIKSGGGGSEVQLVESGGGLVQPGGSLRLSCAASGFTFSTYAMNWV | 328D49 (chain 1) |
| | RQAPGKGLEWVGRIRSKANNYATYYADSVKGRFTISRDDSKNTLYLQM | 328D50 (chain 1) |
| | NSLRAEDTAVYYCVRHGNFGDSYVSWFEYWGQGTLVTVSSGGGGSG | |
| | GGGSGGGGSQAVVTQEPSLTVSPGGTVTLTCGSSTGAVTTSNYANWV | |
| | QQKPGKSPRGLIGGTNKRAPGVPARFSGSLLGGKAALTISGAQPEDEA | |
| | DYYCALWYSNHWVFGGGTKLTVLEPKSSDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKTI | |
| | SKAKGQPREPQVYTLPPCRKKLTKNQVSLTCLVKGFYPSDIAVEWESN | |
| | GQPENNYKTTPPVLKSDGSFFLYSKLTVDKSRWQQGNVFSCSVMHEAL | |
| | HNHYTQKSLSLSPGK* | |
| 135 | QVQLVQSGAEVKKPGASVKVSCKASGYTFTSYYIHWVRQAPGQGLEWI | 328D18 (chain 2) |
| | GSIYPGNVNTNYNEKFKDRATLTVDTSISTAYMELSRLRSDDTAVYFCT | 328D41 (chain 2) |
| | RSHFGYDWNFDVWGQGTTVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGK* | |
| 136 | QVQLVQSGAEVKKPGASVKVSCKASGYTFTDYFMNWVRQAPGQCLE | 328D41 (chain 1) |
| | WMGVINPYNDITIYNQKFQGRVTMTVDRSTSTVYMELSSLRSEDTAVYY | 328D42 (chain 1) |
| | CAREGVLYDGYYEGAYWGQGTLVTVSSGGGGSGGGGSGGGGSDIQL | 328D51 (chain 1) |
| | TQSPSFLSASVGDRVTITCKASQNVGIAVAWYQQKPGKAPKLLIYAASN | 328D52 (chain 1) |
| | RYTGVPSRFSGSGSGTEFTLTISSLQPEDFATYYCQQYSTYPYTFGCGT | 328D53 (chain 1) |
| | KLEIKSGGGGSEVQLVESGGGLVQPGGSLRLSCAASGFTFSTYAMNWV | 328D54 (chain 1) |
| | RQAPGKGLEWVGRIRSKYNNYATYYADSVKGRFTISRDDSKNTLYLQM | 328D55 (chain 1) |
| | NSLRAEDTAVYYCVRHGNFGDSYVSWFEYWGQGTLVTVSSGGGGSG | |
| | GGGSGGGGSQAVVTQEPSLTVSPGGTVTLTCGSSTGAVTTSNYANWV | |
| | QQKPGKSPRGLIGGTNKRAPGVPARFSGSLLGGKAALTISGAQPEDEA | |
| | DYYCALWYSNHWVFGGGTKLTVLEPKSSDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKTI | |
| | SKAKGQPREPQVYTLPPCRKKLTKNQVSLTCLVKGFYPSDIAVEWESN | |
| | GQPENNYKTTPPVLKSDGSFFLYSKLTVDKSRWQQGNVFSCSVMHEAL | |
| | HNHYTQKSLSLSPGK* | |
| 137 | QVQLVQSGAEVKKPGASVKVSCKASGYTFTNSGMNWVRQAPGQCLE | 328C8 (chain 1) |
| | WMGWINTNTGEPTFAEEFRGRVTMTRDTSISTAYMELSRLRSDDTAVY | |
| | YCARYYYGNSFAYWGQGTLVTVSSGGGGSGGGGSGGGGSDIVMTQS | |
| | PDSLAVSLGERATINCKSSQSLLNSGNQKNYLTWYQQKPGQPPKLLIY | |
| | WASTRESGVPDRFSGSGSGTDFTLTISSLQAEDVAVYYCQNNYFYPLT | |
| | FGCGTKVEIKSGGGGSEVQLVESGGGLVQPGGSLRLSCAASGFTFSTY | |
| | AMNWVRQAPGKGLEWVGRIRSKYNNYATYYADSVKGRFTISRDDSKN | |
| | TLYLQMNSLRAEDTAVYYCVRHGNFGDSYVSWFEYWGQGTLVTVSSG | |
| | GGGSGGGGSGGGGSQAVVTQEPSLTVSPGGTVTLTCGSSTGAVTTSN | |
| | YANWVQQKPGKSPRGLIGGTNKRAPGVPARFSGSLLGGKAALTISGAQ | |
| | PEDEADYYCALWYSNHWVFGGGTKLTVLEPKSSDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALGAP | |
| | IEKTISKAKGQPREPQVYTLPPCRKKLTKNQVSLTCLVKGFYPSDIAVEW | |
| | ESNGQPENNYKTTPPVLKSDGSFFLYSKLTVDKSRWQQGNVFSCSVM | |
| | HEALHNHYTQKSLSLSPGK* | |
| 138 | DIVMTQSPDSLAVSLGERATINCKSSQSLLNSGNQKNYLTWYQQKPGQ | 328C12 (chain 3) |
| | PPKLLIYWASTRESGVPDRFSGSGSGTDFTLTISSLQAEDVAVYYCQNN | 328C13 (chain 3) |
| | YFYPLTFGGGTKVEIKRTVAAPSVFIFPPSDEQLKSGTASVVCLLNNFYP | 328C14 (chain 3) |
| | REAKVQWKVDNALQSGNSQESVTEQDSKDSTYSLSSTLTLSKADYEKH | 328C15 (chain 3) |
| | KVYACEVTHQGLSSPVTKSFNRGEC* | 328C18 (chain 3) |
| | | 328C19 (chain 3) |
| | | 328C20 (chain 3) |
| 139 | QVQLVQSGAEVKKPGASVKVSCKASGYTFTNSGMNWVRQAPGQCLE | 328C9 (chain 1) |
| | WMGWINTNTGEPTFAEEFRGRVTMTRDTSISTAYMELSRLRSDDTAVY | |
| | YCARYYYGNSFAYWGQGTLVTVSSGGGGSGGGGSGGGGSDIVMTQS | |
| | PDSLAVSLGERATINCKSSQSLLNSGNQKNYLTWYQQKPGQPPKLLIY | |
| | WASTRESGVPDRFSGSGSGTDFTLTISSLQAEDVAVYYCQNNYFYPLT | |
| | FGCGTKVEIKSGGGGSEVQLVESGGGLVQPGGSLRLSCAASGFTFSTY | |
| | AMNWVRQAPGKGLEWVGRIRSKANNYATYYADSVKGRFTISRDDSKN | |
| | TLYLQMNSLRAEDTAVYYCVRHGNFGDSYVSWFEYWGQGTLVTVSSG | |
| | GGGSGGGGSGGGGSQAVVTQEPSLTVSPGGTVTLTCGSSTGAVTTSN | |
| | YANWVQQKPGKSPRGLIGGTNKRAPGVPARFSGSLLGGKAALTISGAQ | |
| | PEDEADYYCALWYSNHWVFGGGTKLTVLEPKSSDKTHTCPPCPAPEAA | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALGAP | |
| | IEKTISKAKGQPREPQVYTLPPCRKKLTKNQVSLTCLVKGFYPSDIAVEW | |
| | ESNGQPENNYKTTPPVLKSDGSFFLYSKLTVDKSRWQQGNVFSCSVM | |
| | HEALHNHYTQKSLSLSPGK* | |
| 140 | QVQLVQSGAEVKKPGASVKVSCKASGYTFTSYYIHWVRQAPGQGLEWI | 328C12 (chain 1) |
| | GSIYPGNVNTNYNEKFKDRATLTVDTSISTAYMELSRLRSDDTAVYFCT | |
| | RSHYGYDFNFDVWGQGTTVTVSSGGGGSGGGGSGGGGSGGGGSDI | |
| | QMTQSPSSLSASVGDRVTITCHASQNIYVWLNWYQQKPGKAPKLLIYKA | |
| | SNLHTGVPSRFSGSGSGTDFTLTISSLQPEDFATYYCQQGQTYPYTFG | |
| | GGTKVEIKSGGGGSEVQLVESGGGLVQPGGSLRLSCAASGFTFSTYAM | |
| | NWVRQAPGKGLEWVGRIRSKYNNYATYYADSVKGRFTISRDDSKNTLY | |
| | LQMNSLRAEDTAVYYCVRHGNFGDSYVSWFEYWGQGTLVTVSSGKPG | |
| | SGKPGSGKPGSGKPGSQAVVTQEPSLTVSPGGTVTLTCGSSTGAVTTS | |
| | NYANWVQQKPGKSPRGLIGGTNKRAPGVPARFSGSLLGGKAALTISGA | |
| | QPEDEADYYCALWYSNHWVFGGGTKLTVLEPKSSDKTHTCPPCPAPE | |
| | AAGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALG | |
| | APIEKTISKAKGQPREPQVYTLPPCRKKLTKNQVSLTCLVKGFYPSDIAV | |
| | EWESNGQPENNYKTTPPVLKSDGSFFLYSKLTVDKSRWQQGNVFSCS | |
| | VMHEALHNHYTQKSLSLSPGK* | |
| 141 | QVQLVQSGAEVKKPGASVKVSCKASGYTFTNSGMNWVRQAPGQGLE | 328C12 (chain 2) |
| | WMGWINTNTGEPTFAEEFRGRVTMTRDTSISTAYMELSRLRSDDTAVY | 328C13 (chain 2) |
| | YCARYYYGNSFAYWGQGTLVTVSSASTKGPSVFPLAPSSKSTSGGTAA | 328C20 (chain 2) |
| | LGCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGK* | |
| 142 | QVQLVQSGAEVKKPGASVKVSCKASGYTFTSYYIHWVRQAPGQGLEWI | 328C13 (chain 1) |
| | GSIYPGNVNTNYNEKFKDRATLTVDTSISTAYMELSRLRSDDTAVYFCT | |
| | RSHYGYDFNFDVWGQGTTVTVSSGGGGSGGGGSGGGGSGGGGSDI | |
| | QMTQSPSSLSASVGDRVTITCHASQNIYVWLNWYQQKPGKAPKLLIYKA | |
| | SNLHTGVPSRFSGSGSGTDFTLTISSLQPEDFATYYCQQGQTYPYTFG | |
| | GGTKVEIKSGGGGSEVQLVESGGGLVQPGGSLRLSCAASGFTFSTYAM | |
| | NWVRQAPGKGLEWVGRIRSKANNYATYYADSVKGRFTISRDDSKNTLY | |
| | LQMNSLRAEDTAVYYCVRHGNFGDSYVSWFEYWGQGTLVTVSSGKPG | |
| | SGKPGSGKPGSGKPGSQAVVTQEPSLTVSPGGTVTLTCGSSTGAVTTS | |
| | NYANWVQQKPGKSPRGLIGGTNKRAPGVPARFSGSLLGGKAALTISGA | |
| | QPEDEADYYCALWYSNHWVFGGGTKLTVLEPKSSDKTHTCPPCPAPE | |
| | AAGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALG | |
| | APIEKTISKAKGQPREPQVYTLPPCRKKLTKNQVSLTCLVKGFYPSDIAV | |
| | EWESNGQPENNYKTTPPVLKSDGSFFLYSKLTVDKSRWQQGNVFSCS | |
| | VMHEALHNHYTQKSLSLSPGK* | |
| 143 | EVQLVESGGGLVQPGGSLRLSCAASGFTFSTYAMNWVRQAPGKGLEW | 328C14 (chain 1) |
| | VGRIRSKYNNYATYYADSVKGRFTISRDDSKNTLYLQMNSLRAEDTAVY | |
| | YCVRHGNFGDSYVSWFEYWGQGTLVTVSSGGGGSGGGGSGGGGSQ | |
| | AVVTQEPSLTVSPGGTVTLTCGSSTGAVTTSNYANWVQQKPGKSPRGL | |
| | IGGTNKRAPGVPARFSGSLLGGKAALTISGAQPEDEADYYCALWYSNH | |
| | WVFGGGTKLTVLEPKSSDKTHTCPPCPAPEAAGGPSVFLFPPKPKDTL | |
| | MISRTPEVTCVVVDVSHEDPEVKFNWYVDGVEVHNAKTKPREEQYNST | |
| | YRVVSVLTVLHQDWLNGKEYKCKVSNKALGAPIEKTISKAKGQPREPQV | |
| | YTLPPCRKKLTKNQVSLTCLVKGFYPSDIAVEWESNGQPENNYKTTPPV | |
| | LKSDGSFFLYSKLTVDKSRWQQGNVFSCSVMHEALHNHYTQKSLSLSP | |
| | GK* | |
| 144 | QVQLVQSGAEVKKPGASVKVSCKASGYTFTNSGMNWVRQAPGQGLE | 328C14 (chain 2) |
| | WMGWINTNTGEPTFAEEFRGRVTMTRDTSISTAYMELSRLRSDDTAVY | 328C15 (chain 2) |
| | YCARYYYGNSFAYWGQGTLVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGGGGGGGGGSQVQLVQSGAEVKKPGASVKVSC | |
| | KASGYTFTSYYIHWVRQAPGQGLEWIGSIYPGNVNTNYNEKFKDRATLT | |
| | VDTSISTAYMELSRLRSDDTAVYFCTRSHYGYDFNFDVWGQGTTVTVS | |
| | SGGGGSGGGGSGGGGSGGGGSDIQMTQSPSSLSASVGDRVTITCHA | |
| | SQNIYVWLNWYQQKPGKAPKLLIYKASNLHTGVPSRFSGSGSGTDFTLT | |
| | ISSLQPEDFATYYCQQGQTYPYTFGGGTKVEIK* | |
| 145 | EVQLVESGGGLVQPGGSLRLSCAASGFTFSTYAMNWVRQAPGKGLEW | 328C15 (chain 1) |
| | VGRIRSKANNYATYYADSVKGRFTISRDDSKNTLYLQMNSLRAEDTAVY | 328C19 (chain 1) |
| | YCVRHGNFGDSYVSWFEYWGQGTLVTVSSGGGGGGGGSGGGGSQ | |
| | AVVTQEPSLTVSPGGTVTLTCGSSTGAVTTSNYANWVQQKPGKSPRGL | |
| | IGGTNKRAPGVPARFSGSLLGGKAALTISGAQPEDEADYYCALWYSNH | |
| | WVFGGGTKLTVLEPKSSDKTHTCPPCPAPEAAGGPSVFLFPPKPKDTL | |
| | MISRTPEVTCVVVDVSHEDPEVKFNWYVDGVEVHNAKTKPREEQYNST | |
| | YRVVSVLTVLHQDWLNGKEYKCKVSNKALGAPIEKTISKAKGQPREPQV | |
| | YTLPPCRKKLTKNQVSLTCLVKGFYPSDIAVEWESNGQPENNYKTTPPV | |
| | LKSDGSFFLYSKLTVDKSRWQQGNVFSCSVMHEALHNHYTQKSLSLSP | |
| | GK* | |
| 146 | GGGGS | exemplary linker |
| 147 | GKPGS | exemplary linker |
| 148 | GEPGS | exemplary linker |
| 149 | GGPGS | exemplary linker |
| 150 | QVQLVQSGAEVKKPGSSVKVSCKASGYTFTSYWMNWVRQAPGQCLE | 328M32 (chain 1) |
| | WMGMIHPSDSETRLNQKFKDRVTITADKSTSTAYMELSSLRSEDTAVYY | |
| | CARQGIITSVQEFAYWGQGTLVTVSSGGGGSGGGGSGGGGSGGGGSI | |
| | QMTQSPSSLSASVGDRVTITCSASSSVNYIYWYQQKPGKAPKLLIYRTS | |
| | NLASGVPSRFSGSGSGTDFTLTISSLQPEDFATYYCQQYHSYPLTFGCG | |
| | TKVEIKSGGGGSEVQLVESGGGLVQPGGSLRLSCAASGFTFSTYAMN | |
| | WVRQAPGKGLEWVGRIRSKYNNYATYYADSVKGRFTISRDDSKNTLYL | |
| | QMNSLRAEDTAVYYCVRHGNFGDSYVSWFEYWGQGTLVTVSSGGGG | |
| | SGGGGSGGGGSQAVVTQEPSLTVSPGGTVTLTCGSSTGAVTTSNYAN | |
| | WVQQKPGKSPRGLIGGTNKRAPGVPARFSGSLLGGKAALTISGAQPED | |
| | EADYYCALWYSNHWVFGGGTKLTVLEPKSSDKTHTCPPCPAPEAAGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALGAPIE | |
| | KTISKAKGQPREPQVYTLPPCRKKLTKNQVSLTCLVKGFYPSDIAVEWE | |
| | SNGQPENNYKTTPPVLKSDGSFFLYSKLTVDKSRWQQGNVFSCSVMH | |
| | EALHNHYTQKSLSLSPGK* | |
| 151 | QVQLQQWGAGLLKPSETLSLTCAVYGGSFSGYYWSWIRQPPGKCLEW | 328M56s (Chain 1) |
| | IGDIDASGSTKYNPSLKSRVTISLDTSKNQFSLKLNSVTAADTAVYFCAR | |
| | KKYSTVWSYFDNWGQGTLVTVSSGGGGSGGGGSGGGGSSYELTQPS | |
| | SVSVPPGQTASITCSGDKLGDKYASWYQQKPGQSPVLVIYQDRKRPSG | |
| | VPERFSGSNSGNTATLTISGTQAMDEADYYCQAWGSSTAVFGCGTKLT | |
| | VLSGGGGSEVQLVESGGGLVQPGGSLRLSCAASGFTFSTYAMNWVRQ | |
| | APGKGLEWVGRIRSKANNYATYYADSVKGRFTISRDDSKNTLYLQMNS | |
| | LRAEDTAVYYCVRHGNFGDSYVSWFEYWGQGTLVTVSSGGGGSGGG | |
| | GSGGGGSQAVVTQEPSLTVSPGGTVTLTCGSSTGAVTTSNYANWVQQ | |
| | KPGKSPRGLIGGTNKRAPGVPARFSGSLLGGKAALTISGAQPEDEADYY | |
| | CALWYSNHWVFGGGTKLTVLEPKSSDKTHTCPPCPAPEAAGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALGAPIEKTISKAK | |
| | GQPREPQVYTLPPCRKKLTKNQVSLTCLVKGFYPSDIAVEWESNGQPE | |
| | NNYKTTPPVLKSDGSFFLYSKLTVDKSRWQQGNVFSCSVMHEALHNHY | |
| | TQKSLSLSPGK* | |
| 152 | QVQLQQWGAGLLKPSETLSLTCAVYGGSFSGYYWSWIRQPPGKCLEW | 328M58s (Chain 1) |
| | IGDIDASGSTKYNPSLKSRVTISLDTSKNQFSLKLNSVTAADTAVYFCAR | |
| | KKYSTVWSYFDNWGQGTLVTVSSGGGGSGGGGSGGGGSSYELTQPS | |
| | SVSVPPGQTASITCSGDKLGDKYASWYQQKPGQSPVLVIYQDRKRPSG | |
| | VPERFSGSNSGNTATLTISGTQAMDEADYYCQAWGSSTAVFGCGTKLT | |
| | VLSGGGGSEVQLVESGGGLVQPGGSLRLSCAASGFTFSTYAMNWVRQ | |
| | APGKGLEWVGRIRSKYNNYATYYADSVKGRFTISRDDSKNTLYLQMNS | |
| | LRAEDTAVYYCVRHGNFGDSYVSWFDYWGQGTLVTVSSGGGGSGGG | |
| | GSGGGGSQAVVTQEPSLTVSPGGTVTLTCGSSTGAVTTSNYANWVQQ | |
| | KPGKSPRGLIGGTNKRAPGVPARFSGSLLGGKAALTISGAQPEDEADYY | |
| | CALWYSNHWVFGGGTKLTVLEPKSSDKTHTCPPCPAPEAAGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALGAPIEKTISKAK | |
| | GQPREPQVYTLPPCRKKLTKNQVSLTCLVKGFYPSDIAVEWESNGQPE | |
| | NNYKTTPPVLKSDGSFFLYSKLTVDKSRWQQGNVFSCSVMHEALHNHY | |
| | TQKSLSLSPGK* | |
| 153 | QVQLVQSGAEVKKPGASVKVSCKASGYTFTSYYIHWVRQAPGQGLEWI | 328M62 (chain 2) |
| | GSIYPGSVNTNYNEKFKDRATLTVDTSISTAYMELSRLRSDDTAVYFCTR | 328D47 (chain 2) |
| | SHFGYDWNFDVWGQGTTVTVSSASTKGPSVFPLAPSSKSTSGGTAAL | 328D55 (chain 2) |
| | GCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGK* | |
| 154 | EVQLLESGGGLVQPGGSLRLSCAASGFTFSSYGMHWVRQAPGKGLEW | 328D48 (Chain 2) |
| | VAVISHHGSSKYYADSVKGRFTISRDNSKNTLYLQMNSLRAEDTAVYYC | 328D51 (Chain 2) |
| | ARDWFFYLFDYWGQGTLVTVSSASTKGPSVFPLAPSSKSTSGGTAALG | |
| | CLVKDYFPEPVTVSWNSGALTSGVHTFPAVLQSSGLYSLSSVVTVPSSS | |
| | LGTQTYICNVNHKPSNTKVDKRVEPKSCDKTHTCPPCPAPEAAGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALGAPIEKTISK | |
| | AKGQPREPQVCTLPPSRDELTKNQVSLTCLVEGFYPSDIAVEWESNGQ | |
| | PENNYKTTPPVLDSDGSFFLYSDLTVDKSRWQQGNVFSCSVMHEALHN | |
| | HYTQESLSLSPGGGGGSGGGGSQVQLVQSGAEVKKPGASVKVSCKAS | |
| | GYTFTSYYIHWVRQAPGQGLEWIGSIYPGNVNTNYNEKFKDRATLTVDT | |
| | SISTAYMELSRLRSDDTAVYFCTRSHFGYDWNFDVWGQGTTVTVSSG | |
| | GGGSGGGGSGGGGSGGGGSDIQMTQSPSSLSASVGDRVTITCHASQ | |
| | NIYVWLNWYQQKPGKAPKLLIYKASNLHTGVPSRFSGSGSGTDFTLTIS | |
| | SLQPEDFATYYCQQGQTYPYTFGGGTKVEIK* | |
| 155 | DIQMTQSPSSLSASVGDRVTITCKSSQSLLHSDAKTFLYWYQQKPGKAP | 328D48 (Chain 3) |
| | KLLIYEVSNRFSGVPSRFSGSGSGTDFTLTISSLQPEDFATYYCLQGERL | 328D49 (Chain 3) |
| | PFTFGQGTKVEIKRTVAAPSVFIFPPSDEQLKSGTASVVCLLNNFYPREA | 328D50 (Chain 3) |
| | KVQWKVDNALQSGNSQESVTEQDSKDSTYSLSSTLTLSKADYEKHKVY | 328D51 (Chain 3) |
| | ACEVTHQGLSSPVTKSFNRGEC* | 328D52 (Chain 3) |
| | | 328D53 (Chain 3) |
| | | 328D54 (Chain 3) |
| 156 | EVQLLESGGGLVQPGGSLRLSCAASGFTFSSYGMHWVRQAPGKGLEW | 328D49 (Chain 2) |
| | VAVISHHGSSKYYADSVKGRFTISRDNSKNTLYLQMNSLRAEDTAVYYC | 328D52 (Chain 2) |
| | ARDWFFYLFDYWGQGTLVTVSSASTKGPSVFPLAPSSKSTSGGTAALG | |
| | CLVKDYFPEPVTVSWNSGALTSGVHTFPAVLQSSGLYSLSSVVTVPSSS | |
| | LGTQTYICNVNHKPSNTKVDKRVEPKSCDKTHTCPPCPAPEAAGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALGAPIEKTISK | |
| | AKGQPREPQVCTLPPSRDELTKNQVSLTCLVEGFYPSDIAVEWESNGQ | |
| | PENNYKTTPPVLDSDGSFFLYSDLTVDKSRWQQGNVFSCSVMHEALHN | |
| | HYTQESLSLSPGGGGGSGGGGSQVQLVQSGAEVKKPGASVKVSCKAS | |
| | GYTFTSYYIHWVRQAPGQGLEWIGSIYPGSVNTNYNEKFKDRATLTVDT | |
| | SISTAYMELSRLRSDDTAVYFCTRSHFGYDWNFDVWGQGTTVTVSSG | |
| | GGGSGGGGSGGGGSGGGGSDIQMTQSPSSLSASVGDRVTITCHASQ | |
| | NIYVWLNWYQQKPGKAPKLLIYKASNLHTGVPSRFSGSGSGTDFTLTIS | |
| | SLQPEDFATYYCQQGQTYPYTFGGGTKVEIK* | |
| 157 | EVQLLESGGGLVQPGGSLRLSCAASGFTFSSYGMHWVRQAPGKGLEW | 328D50 (Chain 2) |
| | VAVISHHGSSKYYADSVKGRFTISRDNSKNTLYLQMNSLRAEDTAVYYC | 328D53 (Chain 2) |
| | ARDWFFYLFDYWGQGTLVTVSSASTKGPSVFPLAPSSKSTSGGTAALG | |
| | CLVKDYFPEPVTVSWNSGALTSGVHTFPAVLQSSGLYSLSSVVTVPSSS | |
| | LGTQTYICNVNHKPSNTKVDKRVEPKSCDKTHTCPPCPAPEAAGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALGAPIEKTISK | |
| | AKGQPREPQVCTLPPSRDELTKNQVSLTCLVEGFYPSDIAVEWESNGQ | |
| | PENNYKTTPPVLDSDGSFFLYSDLTVDKSRWQQGNVFSCSVMHEALHN | |
| | HYTQESLSLSPGGGGGSGGGGSQVQLVQSGAEVKKPGASVKVSCKAS | |
| | GYTFTSYYIHWVRQAPGQGLEWIGSIYPGNVNTNYNEKFKDRATLTVDT | |
| | SISTAYMELSRLRSDDTAVYFCTRSHYGYDFNFDVWGQGTTVTVSSGG | |
| | GGSGGGGSGGGGSGGGGSDIQMTQSPSSLSASVGDRVTITCHASQNI | |
| | YVWLNWYQQKPGKAPKLLIYKASNLHTGVPSRFSGSGSGTDFTLTISSL | |
| | QPEDFATYYCQQGQTYPYTFGGGTKVEIK* | |
| 158 | EVQLLESGGGLVQPGGSLRLSCAASGFTFSSYGMHWVRQAPGKGLEW | 328D54 (Chain 2) |
| | VAVISHHGSSKYYADSVKGRFTISRDNSKNTLYLQMNSLRAEDTAVYYC | |
| | ARDWFFYLFDYWGQGTLVTVSSASTKGPSVFPLAPSSKSTSGGTAALG | |
| | CLVKDYFPEPVTVSWNSGALTSGVHTFPAVLQSSGLYSLSSVVTVPSSS | |
| | LGTQTYICNVNHKPSNTKVDKRVEPKSCDKTHTCPPCPAPEAAGGPSV | |
| | FLFPPKPKDTLMISRTPEVTCVVVDVSHEDPEVKFNWYVDGVEVHNAKT | |
| | KPREEQYNSTYRVVSVLTVLHQDWLNGKEYKCKVSNKALGAPIEKTISK | |
| | AKGQPREPQVCTLPPSRDELTKNQVSLTCLVEGFYPSDIAVEWESNGQ | |
| | PENNYKTTPPVLDSDGSFFLYSDLTVDKSRWQQGNVFSCSVMHEALHN | |
| | HYTQESLSLSPGGGGGSGGGGSQVQLVQSGAEVKKPGASVKVSCKAS | |
| | GYTFTSYYIHWVRQAPGQGLEWIGSIYPGNVNTNYNEKFKDRATLTVDT | |
| | SISTAYMELSRLRSDDTAVYFCTRSHYGLDANFDVWGQGTTVTVSSGG | |
| | GGSGGGGSGGGGSGGGGSDIQMTQSPSSLSASVGDRVTITCHASQNI | |
| | YVWLNWYQQKPGKAPKLLIYKASNLHTGVPSRFSGSGSGTDFTLTISSL | |
| | QPEDFATYYCQQGQTYPYTFGGGTKVEIK* | |
| 159 | EVQLVESGGGLVQPGGSLRLSCAASGFTFSTYAMNWVRQAPGKGLEW | 328C18 (chain 1) |
| | VGRIRSEANNYATYYADSVKGRFTISRDDSKNTLYLQMNSLRAEDTAVY | |
| | YCVRHGNFGDSYVSWFEYWGQGTLVTVSSGGGGSGGGGSGGGGSQ | |
| | AVVTQEPSLTVSPGGTVTLTCGSSTGAVTTSNYANWVQQKPGKSPRGL | |
| | IGGTNKRAPGVPARFSGSLLGGKAALTISGAQPEDEADYYCALWYSNH | |
| | WVFGGGTKLTVLEPKSSDKTHTCPPCPAPEAAGGPSVFLFPPKPKDTL | |
| | MISRTPEVTCVVVDVSHEDPEVKFNWYVDGVEVHNAKTKPREEQYNST | |
| | YRVVSVLTVLHQDWLNGKEYKCKVSNKALGAPIEKTISKAKGQPREPQV | |
| | YTLPPCRKKLTKNQVSLTCLVKGFYPSDIAVEWESNGQPENNYKTTPPV | |
| | LKSDGSFFLYSKLTVDKSRWQQGNVFSCSVMHEALHNHYTQKSLSLSP | |
| | GK* | |
| 160 | QVQLVQSGAEVKKPGASVKVSCKASGYTFTNSGMNWVRQAPGQGLE | 328C18 (chain 2) |
| | WMGWINTNTGEPTFAEEFRGRVTMTRDTSISTAYMELSRLRSDDTAVY | 328C19 (chain 2) |
| | YCARYYYGNSFAYWGQGTLVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPEAAGGP | |
| | SVFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALGAPIEKT | |
| | ISKAKGQPREPQVCTLPPSRDELTKNQVSLTCLVEGFYPSDIAVEWESN | |
| | GQPENNYKTTPPVLDSDGSFFLYSDLTVDKSRWQQGNVFSCSVMHEA | |
| | LHNHYTQESLSLSPGGGGGSGGGGSQVQLVQSGAEVKKPGASVKVSC | |
| | KASGYTFTSYYIHWVRQAPGQGLEWIGSIYPGSVNTNYNEKFKDRATLT | |
| | VDTSISTAYMELSRLRSDDTAVYFCTRSHFGYDWNFDVWGQGTTVTVS | |
| | SGGGGSGGGGSGGGGSGGGGSDIQMTQSPSSLSASVGDRVTITCHA | |
| | SQNIYVWLNWYQQKPGKAPKLLIYKASNLHTGVPSRFSGSGSGTDFTLT | |
| | ISSLQPEDFATYYCQQGQTYPYTFGGGTKVEIK* | |
| 161 | QVQLVQSGAEVKKPGASVKVSCKASGYTFTSYYIHWVRQAPGQGLEWI | 328C20 (chain 1) |
| | GSIYPGSVNTNYNEKFKDRATLTVDTSISTAYMELSRLRSDDTAVYFCTR | |
| | SHFGYDWNFDVWGQGTTVTVSSGGGGSGGGGSGGGGSGGGGSDIQ | |
| | MTQSPSSLSASVGDRVTITCHASQNIYVWLNWYQQKPGKAPKLLIYKAS | |
| | NLHTGVPSRFSGSGSGTDFTLTISSLQPEDFATYYCQQGQTYPYTFGG | |
| | GTKVEIKSGGGGSEVQLVESGGGLVQPGGSLRLSCAASGFTFSTYAMN | |
| | WVRQAPGKGLEWVGRIRSKANNYATYYADSVKGRFTISRDDSKNTLYL | |
| | QMNSLRAEDTAVYYCVRHGNFGDSYVSWFEYWGQGTLVTVSSGKPG | |
| | SGKPGSGKPGSGKPGSQAVVTQEPSLTVSPGGTVTLTCGSSTGAVTTS | |
| | NYANWVQQKPGKSPRGLIGGTNKRAPGVPARFSGSLLGGKAALTISGA | |
| | QPEDEADYYCALWYSNHWVFGGGTKLTVLEPKSSDKTHTCPPCPAPE | |
| | AAGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALG | |
| | APIEKTISKAKGQPREPQVYTLPPCRKKLTKNQVSLTCLVKGFYPSDIAV | |
| | EWESNGQPENNYKTTPPVLKSDGSFFLYSKLTVDKSRWQQGNVFSCS | |
| | VMHEALHNHYTQKSLSLSPGK* | |

What is claimed is:

1. A multi-specific molecule, comprising:
a) a first targeting domain that specifically binds to human CD3 with a binding affinity that equals to, or is greater than, 1 nmol/L;
b) a second targeting domain that specifically binds to human CD28 with a binding affinity that equals to, or is greater than, 100 nmol/L;
c) a third targeting domain that specifically binds to human Muc17, or human DLL3, or human CLDN18.2; and
d) an Fc fragment that lacks antibody-dependent cellular cytotoxicity.

2. The multi-specific molecule of claim 1, wherein the third targeting domain specifically binds to human Muc17.

3. The multi-specific molecule of claim 2, wherein the third targeting domain comprises
(1) an HCDR1 of SEQ ID NO:1, an HCDR2 of SEQ ID NO:2, an HCDR3 of SEQ ID NO: 3, an LCDR1 of SEQ ID NO:4, an LCDR2 of SEQ ID NO:5, and an LCDR3 of SEQ ID NO: 6, or
(2) an HCDR1 of SEQ ID NO:27, an HCDR2 of SEQ ID NO:28, an HCDR3 of SEQ ID NO: 29, an LCDR1 of SEQ ID NO:30, an LCDR2 of SEQ ID NO:31, and an LCDR3 of SEQ ID NO:32,

4. The multi-specific molecule of claim 2, wherein the second targeting domain comprises:
(1) an HCDR1 of SEQ ID NO: 107, an HCDR2 of SEQ ID NO: 108, an HCDR3 of SEQ ID NO: 124, an LCDR1 of SEQ ID NO: 110, an LCDR2 of SEQ ID NO: 111, and an LCDR3 of SEQ ID NO:112; or
(2) an HCDR1 of SEQ ID NO: 107, an HCDR2 of SEQ ID NO: 120, an HCDR3 of SEQ ID NO:115, an LCDR1 of SEQ ID NO:110, an LCDR2 of SEQ ID NO: 111, and an LCDR3 of SEQ ID NO:112.

5. The multi-specific molecule of claim 2, wherein the first targeting domain comprises:
(1) an HCDR1 of SEQ ID NO:93, an HCDR2 of SEQ ID NO:94, an HCDR3 of SEQ ID NO:95, an LCDR1 of SEQ ID NO:96, an LCDR2 of SEQ ID NO:97, and an LCDR3 of SEQ ID NO:98; or
(2) an HCDR1 of SEQ ID NO:93, an HCDR2 of SEQ ID NO:101, an HCDR3 of SEQ ID NO:95, an LCDR1 of SEQ ID NO:96, an LCDR2 of SEQ ID NO:97, and an LCDR3 of SEQ ID NO:98; or
(3) an HCDR1 of SEQ ID NO:93, an HCDR2 of SEQ ID NO:94, an HCDR3 of SEQ ID NO: 105, an LCDR1 of SEQ ID NO:96, an LCDR2 of SEQ ID NO:97, and an LCDR3 of SEQ ID NO:98.

6. The multi-specific molecule of claim 1, wherein the third targeting domain specifically binds to human DLL3

7. The multi-specific molecule of claim 6, wherein the third targeting domain comprises:
(1) an HCDR1 of SEQ ID NO:35, an HCDR2 of SEQ ID NO:36, an HCDR3 of SEQ ID NO:37, an LCDR1 of SEQ ID NO:38, an LCDR2 of SEQ ID NO:39, and an LCDR3 of SEQ ID NO:40; or
(2) an HCDR1 of SEQ ID NO:43, an HCDR2 of SEQ ID NO:44, an HCDR3 of SEQ ID NO:45, an LCDR1 of SEQ ID NO:46, an LCDR2 of SEQ ID NO:47, and an LCDR3 of SEQ ID NO:48.

8. The multi-specific molecule of claim 6, wherein the second targeting domain comprises:
(1) an HCDR1 of SEQ ID NO:107, an HCDR2 of SEQ ID NO:108, an HCDR3 of SEQ ID NO: 124, an LCDR1 of SEQ ID NO:110, an LCDR2 of SEQ ID NO: 111, and an LCDR3 of SEQ ID NO:112; or
(2) an HCDR1 of SEQ ID NO:107, an HCDR2 of SEQ ID NO: 120, an HCDR3 of SEQ ID NO: 115, an LCDR1 of SEQ ID NO:110, an LCDR2 of SEQ ID NO: 111, and an LCDR3 of SEQ ID NO:112.

9. The multi-specific molecule of claim 6, wherein the first targeting domain comprises:
(1) an HCDR1 of SEQ ID NO:93, an HCDR2 of SEQ ID NO:94, an HCDR3 of SEQ ID NO:95, an LCDR1 of SEQ ID NO:96, an LCDR2 of SEQ ID NO:97, and an LCDR3 of SEQ ID NO:98; or
(2) an HCDR1 of SEQ ID NO:93, an HCDR2 of SEQ ID NO: 101, an HCDR3 of SEQ ID NO:95, an LCDR1 of SEQ ID NO:96, an LCDR2 of SEQ ID NO:97, and an LCDR3 of SEQ ID NO:98.

10. The multi-specific molecule of claim 1, wherein the third targeting domain specifically binds to human CLDN18.2.

11. The multi-specific molecule of claim 10, wherein the third targeting domain comprises an HCDR1 of SEQ ID NO:65, an HCDR2 of SEQ ID NO:66, an HCDR3 of SEQ ID NO:67, an LCDR1 of SEQ ID NO:68, an LCDR2 of SEQ ID NO:69, and an LCDR3 of SEQ ID NO:70.

12. The multi-specific molecule of claim 10, wherein the second targeting domain comprises:
(1) an HCDR1 of SEQ ID NO:107, an HCDR2 of SEQ ID NO:108, an HCDR3 of SEQ ID NO: 124, an LCDR1 of SEQ ID NO: 110, an LCDR2 of SEQ ID NO: 111, and an LCDR3 of SEQ ID NO:112; or
(2) an HCDR1 of SEQ ID NO: 107, an HCDR2 of SEQ ID NO: 120, an HCDR3 of SEQ ID NO: 115, an LCDR1 of SEQ ID NO: 110, an LCDR2 of SEQ ID NO: 111, and an LCDR3 of SEQ ID NO:112.

13. The multi-specific molecule of claim 11, wherein the first targeting domain comprises an HCDR1 of SEQ ID NO:93, an HCDR2 of SEQ ID NO: 101 and 103, an HCDR3 of SEQ ID NO:95, an LCDR1 of SEQ ID NO:96, an LCDR2 of SEQ ID NO:97, and an LCDR3 of SEQ ID NO:98.

14. The multi-specific molecule of claim 1, wherein the first targeting domain specifically binds to human CD3 with a binding affinity in the range of 1-2000 nmol/L.

15. The multi-specific molecule of claim 1, wherein the second targeting domain specifically binds to human CD28 with a binding affinity in the range of 100-10000 nmol/L.

16. The multi-specific molecule of claim 1, wherein the first targeting domain specifically binds to human CD3 with a binding affinity in the range of 1-2000 nmol/L and wherein the second targeting domain specifically binds to human CD28 with a binding affinity in the range of 100-10000 nmol/L.

17. The multi-specific molecule of claim 1, wherein the first targeting domain specifically binds to human CD3 with a binding affinity in the range of 1-500 nmol/L and wherein the second targeting domain specifically binds to human CD28 with a binding affinity in the range of 100-2000 nmol/L.

18. The multi-specific molecule of claim 1, wherein each targeting domain comprises at least one antibody fragment selected from the group consisting of single domain antibody (sdAb), a fragment variable (Fv) heterodimer, a single chain Fv (scFv), Fab fragment and combinations thereof.

19. A method of stimulating T-cell activity in a subject, comprising the step of administering to the subject, an effective amount of the multi-specific molecule of claim 1.

20. A method to treating cancer in a subject, comprising the step of administering to the subject, an effective amount of the multi-specific molecule of claim 1.
